(19) United States
(12) Patent Application Publication
(10) Pub. No.: US 2025/0093342 A1
(43) Pub. Date: Mar. 20, 2025

(54) ANALYZING CELL SAMPLES

(71) Applicant: Singular Genomics Systems, Inc., San Diego, CA (US)

(72) Inventor: Eli N. Glezer, Del Mar, CA (US)

(21) Appl. No.: 18/959,243

(22) Filed: Nov. 25, 2024

Related U.S. Application Data
(63) Continuation of application No. 18/741,582, filed on Jun. 12, 2024.
(60) Provisional application No. 63/508,214, filed on Jun. 14, 2023.
(60) Provisional application No. 63/515,042, filed on Jul. 21, 2023.

(51) Int. Cl. G01N 33/53; G01N 33/542

(52) U.S. Cl. CPC G01N 33/5308; G01N 33/542

(57) ABSTRACT

Disclosed herein, inter alia, are methods and compositions useful for detecting multiple biological targets.

### CROSS-REFERENCES TO RELATED APPLICATIONS

This application claims the benefit of U.S. Utility application Ser. No. 18/741,582, filed Jun. 12, 2024, which claims the benefit of U.S. Provisional Application No. 63/508,214, filed Jun. 14, 2023; and U.S. Provisional Application No. 63/515,042, filed Jul. 21, 2023, each of which are incorporated herein by reference in their entirety and for all purposes.

### BACKGROUND

Detecting multiple biomolecules (e.g., organelles, proteins or nucleic acids) in the same cell or tissue section (i.e., in situ) represents an ideal goal for understanding phenotypic and functional architecture of in healthy and diseased subjects. Uncovering the distribution, heterogeneity, spatial gene, and protein co-expression patterns within cells and tissues is vital for understanding how cell co-localization influences tissue development and the spread of diseases such as cancer, which could lead to important new discoveries and therapeutics. The ability to multiplex, that is, detect multiple different analytes in the same sample, remains a significant challenge. Disclosed herein, inter alia, are solutions to these and other problems in the art.

### BRIEF SUMMARY

In an aspect is provided a method of detecting multiple targets. In embodiments, the method includes: (i) contacting a cell or tissue with a probe including a detectable label and a quenching moiety, wherein the detectable label is attached to the probe via a cleavable linker; (ii) binding the probe to a target of the cell or tissue; (iii) removing the quenching moiety and detecting the probe bound to a target; (iv) cleaving the cleavable linker; and repeating steps (i) to (iii) to detect multiple targets. In embodiments, the target is a biomolecule. In embodiments, the biomolecule is a lipid, carbohydrate, peptide, protein, or antigen binding fragment.

In an aspect is provided a method of detecting multiple biomolecules, the method including: (a) contacting a cell or tissue with a first probe and a second probe, thereby forming a first complex and a second complex, wherein the first complex includes the first probe bound to a first biomolecule, wherein the first probe includes a first detectable label attached to the first probe via a first linker; the second complex includes the second probe bound to a second biomolecule, wherein the second probe includes a second detectable label and a quenching moiety, wherein the second detectable label is attached to the second probe via a second linker; and the quenching moiety is attached to the second probe via a first cleavable linker; (b) detecting the first complex; cleaving the first cleavable linker, thereby separating the quenching moiety from the second complex; and (c) detecting the second complex. In embodiments, the first linker includes a cleavable site (i.e., the first linker is a cleavable linker).

In an aspect is provided a cell or tissue including two target complexes, as described herein. In embodiments, the a first target complex includes a first probe bound to a first biomolecule, wherein the first probe includes a first detectable label attached to the first probe via a first linker; and the second target complex includes a second probe bound to a second biomolecule, wherein the second probe includes a second detectable label and a quenching moiety, wherein the second detectable label is attached to the second probe via a second linker; and the quenching moiety is attached to the second probe via a cleavable linker.

In another aspect is provided a kit, including: (i) a first compound having the formula: Ab1-L¹-Dye1; (ii) a second compound having the formula: Ab2-L²-Dye2-L¹-Q¹; (iii) a third compound having the formula: Ab3-L³-Dye3-L²-Q²; wherein, Ab1 is a first antibody; Ab2 is a second antibody; Ab3 is a third antibody; L¹, L² and L³ are orthogonally cleavable linkers; Dye1, Dye2, and Dye3 are independently fluorescent moieties; Q¹ and Q² are quenching moieties, wherein Dye2 and Q1 are a first fluorescent-quencher pair; and Dye3 and Q2 are a second fluorescent-quencher pair.

### BRIEF DESCRIPTION OF THE DRAWINGS

FIG. 1 provides a set of labeled antibodies used in typical cyclic immunofluorescence (CycIF) protocols. Briefly cyclical staining protocols include multiple rounds of staining targets with antibodies, wherein after each round of staining the fluorophores are inactivated or the bound antibodies are removed. This may be repeated N times.

FIGS. 2A-2B provides embodiments of the probes contemplated herein. FIG. 2A provides a set of four detection antibodies (Ab-1, Ab-2, Ab-3, and Ab-4) with serially cleaving quenchers. The first detection antibody includes a dye and a cleavable linker, X₁. The second antibody includes a second cleavable linker X₂, the dye, the first cleavable linker X₁, and a quencher. The third antibody includes a third cleavable linker X₃, the dye, the second cleavable linker X₂, and a quencher. The fourth antibody includes a fourth cleavable linker X₄, the dye, the third cleavable linker X₃, and a quencher. FIG. 2B provides analogous oligonucleotide probes (OPs). The first oligonucleotide probe includes a dye and a cleavable linker, X₁. The second oligonucleotide probe includes a second cleavable linker X₂, the dye, the first cleavable linker X₁, and a quencher. The third oligonucleotide probe includes a third cleavable linker X₃, the dye, the second cleavable linker X₂, and a quencher. The fourth oligonucleotide probe includes a fourth cleavable linker X₄, the dye, the third cleavable linker X₃, and a quencher.

FIG. 3 illustrates an example workflow for detecting multiple targets in a sample using the serial reveal method described herein. The set of probes are all applied to the sample (e.g., a cell) and allowed to bind to their respective targets (e.g., proteins, such as antibodies or cytokines), followed by and the serially revealing the dyes by cleaving off their associated quenchers. For example, in the first cycle, the first dye is detected (i.e., Dye 1 of Ab-1), followed by cleaving the first cleavable linker. The first dye is removed from Ab-1, and the quencher moiety is removed from Ab-2, as a result of cleaving the first cleavable linker (e.g., depicted as X₁ in FIGS. 2A and 2B) and thus enabling detection of Dye 2 from Ab-2. Next, the second cleavable linker is cleaved (e.g., depicted as X₂ in FIGS. 2A and 2B), removing the dye (i.e., Dye 2) from Ab-2 and exposing Dye 3 from Ab-3. Next, the third cleavable linker is cleaved, removing the dye (i.e., Dye 3) from Ab-3 and enabling detection of Dye 4 from Ab-4. This process may be repeated for as many cleavable linkers present in the original probe set. Additionally, this may be expanded to include different dye-sets (e.g., one set of four probes include a first dye, a second set of four probes include a second dye, etc.) to enable greater multiplexing.

FIGS. 4A-4B provide combinatorial sets of probes. FIG. 4A provides two set of probes. The first set of oligonucleotide probes include a first dye and three orthogonal cleavable moieties. The second set of oligonucleotide probes include a second dye and the three orthogonal cleavable moieties. FIG. 4B illustrates three sets of probes, wherein each set shares the same dye color. For example, the first set includes a first dye and two orthogonal cleavable moieties, X₁ and X₂. The second set includes a second dye and the two orthogonal cleavable moieties, X₁ and X₂. Finally, the third set includes a third dye and the two orthogonal cleavable moieties, X₁ and X₂.

FIGS. 5A-5B illustrate embodiments of molecular beacon antibody (MB-Ab) probes. FIG. 5A provides a generic formula for constructing a set of N MB-Ab probes. FIG. 5B probes an example set of MB-Ab probes, wherein the first antibody includes a first cleavable moiety denoted as X₁ (e.g., a first restriction enzyme motif). The second antibody includes the first cleavable moiety X₁ such that upon cleavage the quenching moiety (illustrated as an octagon) is removed to expose the dye (illustrated as a star). The second antibody also includes a second cleavable site, denoted as X₂ (e.g., a second restriction enzyme motif) that may be cleaved to remove the dye from the second antibody and remove the quenching moiety from the third antibody.

FIG. 6 provides an illustration of the sequential collection of information to inform on the structure of a cell and/or tissue. Spectrally distinct dyes are used in the first set, and optionally reused in subsequent sets. For example, the first set includes Alexa Fluor® 532 (emission: 532 nm), Alexa Fluor® 594 (emission: 594 nm), Alexa Fluor® 647 (emission: 647 nm), and Alexa Fluor® 680 (emission: 680 nm) to illuminate the Golgi Apparatus, endoplasmic reticulum, actin, lysosomes, and specific cell surface receptors of a cell. Following cleavage and removal of the fluorophores, the second set of targeting molecules are incubated with the sample cell. The second set can then illuminate the nucleus, nucleoli, mitochondria, nuclear envelop, cell surface receptors, and plasma membrane. The sequential addition of cell paints can continue for N cycles providing additional information about the cell. The resulting images may be computationally processed and overlaid to provide a composite image of the cell and/or tissue.

### DETAILED DESCRIPTION

The aspects and embodiments described herein relate to sequentially revealing and detecting targets.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, made up of “dNTPs,” which do not have a hydroxyl group at the 2′ position, and oligoribonucleotides, made up of “NTPs,” which have a hydroxyl group in the 2′ position. Oligonucleotides also may include derivatives, in which the hydrogen of the hydroxyl group is replaced with an organic group, e.g., an allyl group.

Oligonucleotides, as described herein, typically are capable of forming hydrogen bonds with oligonucleotides having a complementary base sequence. These bases may include the natural bases, such as A, G, C, T, and U, as well as artificial, non-standard or non-natural nucleotides such as iso-cytosine and iso-guanine. As described herein, a first sequence of an oligonucleotide is described as being 100% complementary with a second sequence of an oligonucleotide when the consecutive bases of the first sequence (read 5′-to-3′) follow the Watson-Crick rule of base pairing as compared to the consecutive bases of the second sequence (read 3′-to-5′). An oligonucleotide may include nucleotide substitutions. For example, an artificial base may be used in place of a natural base such that the artificial base exhibits a specific interaction that is similar to the natural base.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, a “platform primer” is a primer oligonucleotide immobilized or otherwise bound to a solid support (i.e. an immobilized oligonucleotide). Examples of platform primers include P7 and P5 primers (i.e., Illumina® platform sequences), or S1 and S2 primers (i.e., Singular Genomics® platform sequences), or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support (e.g., a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

The term “adapter” as used herein refers to any oligonucleotide that can be ligated to a nucleic acid molecule, thereby generating nucleic acid products that can be sequenced on a sequencing platform (e.g., an Illumina™ or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In some embodiments, an adapter is hairpin adapter (also referred to herein as a hairpin). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach a label to the molecule. The use of the term “cleavable linker” is not meant to imply that the whole linker is required to be removed. The cleavage site can be located at a position on the linker that ensures that part of the linker remains attached to the molecule after cleavage.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. In embodiments, the cleavable linker is a divalent linker between a quenching moiety and a biomolecule-specific probe (i.e., a probe as described herein). In embodiments, the cleavable linker is a divalent linker between a fluorescent moiety or detectable label and a biomolecule-specific probe (i.e., a probe as described herein). A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

A photocleavable linker (e.g., including or consisting of an o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of a reducing agent (e.g., tris(3-hydroxypropyl)phosphine). In embodiments, the cleavable linker is a dialkylketal linker (Binaulda S., et al., Chem. Commun., 2013, 49, 2082-2102; Shenoi R. A., et al., J. Am. Chem. Soc., 2012, 134, 14945-14957), an azo linker (Rathod, K. M., et al., Chem. Sci. Tran., 2013, 2, 25-28; Leriche G., et al., Eur. J. Org. Chem., 2010, 23, 4360-64), an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent and the agent that cleaves each cleavable linker is different. In embodiments, an orthogonally is a cleavable linker that following cleavage the two separated entities (e.g., fluorescent dye, bioconjugate reactive group) do not further react and form a new orthogonally cleavable linker.

The term “orthogonal detectable label” or “orthogonal detectable moiety” as used herein refer to a detectable label (e.g., fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy5-Labeled Streptavidin, and R6G-Labeled Dibenzocyclooctyne.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

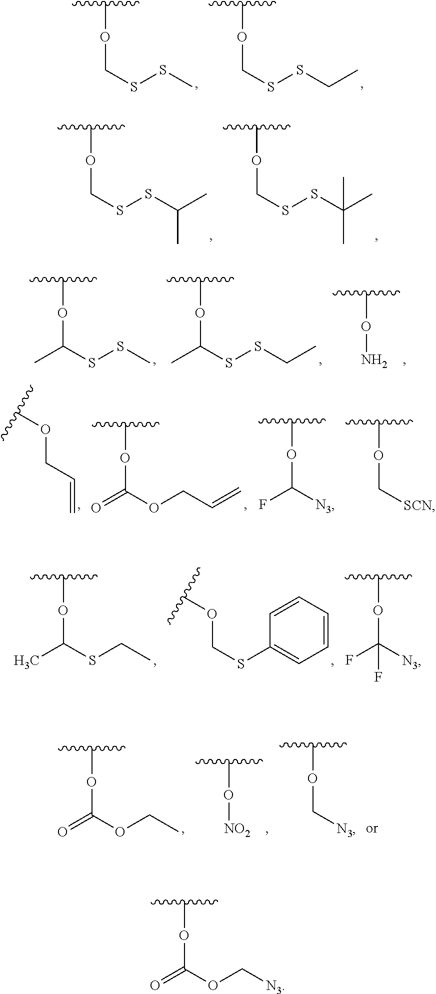

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Atto® dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Atto® dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy®3). In embodiments, the cyanine moiety has 5 methine structures (i.e., cyanine 5 or Cy®5). In embodiments, the cyanine moiety has 7 methine structures (i.e., cyanine 7 or Cy®7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST® or BLAST® 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula: S′ 'S'S S′

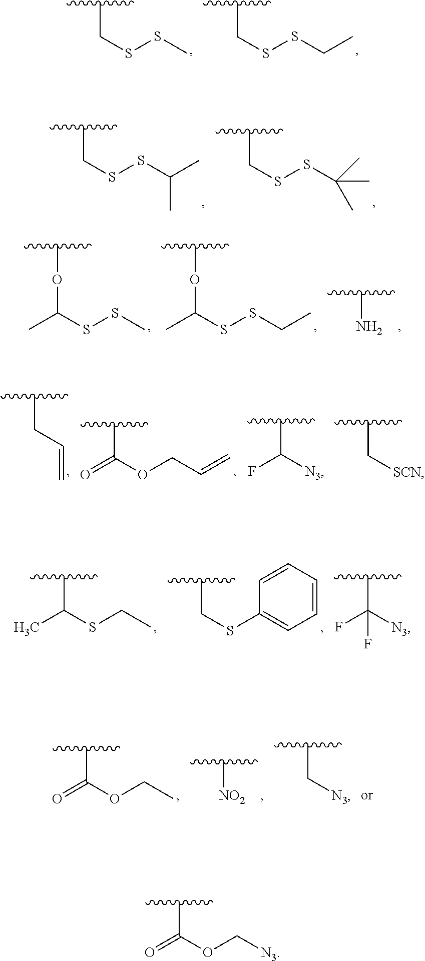

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

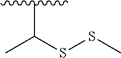 [SMILES: *C(C)SSC]

as described in U.S. Pat. No. 10,738,072, which is incorporated herein by reference for all purposes. For example, a nucleotide including a reversible terminator moiety may be represented by the formula:

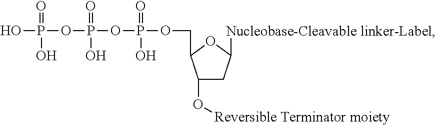

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide. In embodiments, a barcode is a nucleotide, nucleotide sequence, or identifier, that conveys or is capable of conveying information (e.g., information about an analyte in a cell or tissue. The term “barcode” may refer either to a physical barcode molecule (e.g., a nucleic acid barcode molecule) or to its representation in a computer-readable, digital format (e.g., as a string of characters representing the sequence of bases in a nucleic acid barcode molecule).

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

In embodiments, barcodes may include a series of two or more segments or sub-barcodes (e.g., corresponding to “letters” or “code words” in a decoded barcode), each of which may comprise one or more of the subunits or building blocks used to synthesize the physical (e.g., nucleic acid) barcode molecules. For example, a nucleic acid barcode molecule may include two or more barcode segments, each of which includes one or more nucleotides. In embodiments, a barcode includes 1, 2, 3, 4, 5, 6, 7, 8, 9, 10, or more than 10 segments. In embodiments, each segment of a barcode molecule includes at least 1, 2, 3, 4, 5, 6, 7, 8, 9, 10, 12, 14, 16, 18, 20, or more than 20 subunits or building blocks. For example, each segment of a nucleic acid barcode molecule may include 1, 2, 3, 4, 5, 6, 7, 8, 9, 10, 12, 14, 16, 18, 20, or more than 20 nucleotides. In embodiments, two or more of the segments of a barcode may be separated by non-barcode segments, i.e., the segments of a barcode molecule need not be contiguous.

A “digital barcode” (or “digital barcode sequence”) is a representation of a corresponding physical barcode (or target analyte sequence) in a computer-readable, digital format as described above. A digital barcode may comprise one or more “letters” (e.g., 1, 2, 3, 4, 5, 6, 7, 8, 9, 10, 12, 14, 16, 18, 20, or more than 20 letters) or one or more “code words” (e.g., 1, 2, 3, 4, 5, 6, 7, 8, 9, 10, or more than 10 code words), where a “code word” comprises, e.g., 1, 2, 3, 4, 5, 6, 7, 8, 9, 10, 12, 14, 16, 18, 20, or more than 20 letters. In some instances, the sequence of letters or code words in a digital barcode sequence may correspond directly with the sequence of building blocks (e.g., nucleotides) in a physical barcode. In some instances, the sequence of letters or code words in a digital barcode sequence may not correspond directly with the sequence of building blocks in a physical barcode, but rather may comprise, e.g., arbitrary code words that each correspond to a segment of a physical barcode. For example, in some instances, the disclosed methods for decoding and error correction may be applied directly to detecting target analyte sequences (e.g., mRNA sequences) as opposed to detecting target barcodes, and the barcode probes used to detect the target analyte sequences may correspond to letters or code words that have been assigned to specific target analyte sequences but that do not directly correspond to the target analyte sequences

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase™, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR™ DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator™ γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator™ II enzyme from New England Biolabs with D141A/E143A/Y409V/A485L mutations); 3′-amino-dNTPs, 3′-azido-dNTPs and other 3′-modified nucleotides (e.g., NEB Therminator™ III DNA Polymerase with D141A/E143A/L408S/Y409A/P410V mutations, NEB Therminator™ IX DNA polymerase), or 7-phosphate labeled nucleotides (e.g., Therminator™ 7: D141A/E143A/W355A/L408 W/R460A/Q461S/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “ligase” refers to an enzyme that catalyzes the formation of a new phosphodiester bond as a result of joining the 5′-phosphoryl terminus of DNA or RNA to single-stranded 3′-hydroxyl terminus of DNA or RNA. Ligase enzymes can form circular DNA or RNA templates in a non-template driven reaction, and examples of ligase enzymes include, but are not limited to, as CircLigase™, Taq DNA Ligase, HiFi Taq DNA Ligase, T4 ligase, or Ampligase® DNA Ligase.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

The term “polynucleotide fusion” is used in accordance with its plain and ordinary meaning and refers to a polynucleotide formed from the joining of two regions of a reference sequence (e.g., a reference genome) that are not so joined in the reference sequence, thereby creating a fusion junction between the two regions that does not exist in the reference sequence. Polynucleotide fusions can be formed by a number of processes, including interchromosomal translocation, intrachromosomal translocation, and other chromosomal rearrangements (e.g., inversion and duplication). A polynucleotide fusion can involve fusion between two gene sequences, referred to as a “gene fusion” and producing a “fusion gene.” In some cases, a fusion gene is expressed as a fusion transcript (e.g., a fusion mRNA transcript) including sequences of the two genes, or portions thereof.

A “fusion gene” is used in accordance with its ordinary meaning in the art and refers to a hybrid gene, or portion thereof, formed from two previously independent genes, or portions thereof (e.g., in a cell). A “fusion junction” is the point in the fusion gene sequence between the two previously independent genes, or portions thereof. The hybrid gene can result from a translocation, interstitial deletion, and/or chromosomal inversion of a gene or portion of a gene. Chromosomal rearrangements leading to the fusion of coding regions of two genes can result in expression of hybrid proteins. An “exon junction” is the point or location in the fusion gene sequence between the two previously independent exon sequences, or portions thereof.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷M, less than 10⁻⁸M, less than 10⁻⁹M, less than 10⁻¹⁰ M, less than 10⁻¹¹ M, or less than about 10⁻¹² M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

The term “particle” means a small body made of a rigid or semi-rigid material. The body can have a shape characterized, for example, as a sphere, oval, microsphere, or other recognized particle shape whether having regular or irregular dimensions. The particles may in one way or another rest upon a two dimensional surface by magnetic, gravitational, or ionic forces, or by chemical bonding, or by any other means known to those skilled in the art. In further embodiments, the bead may have magnetic properties. Further the beads may have a density that allows them to rest upon a two dimensional surface in solution. Particles may consist of glass, polystyrene, latex, metal, quantum dot, polymers, silica, metal oxides, ceramics, or any other substance suitable for binding to nucleic acids, or chemicals or proteins which can then attach to nucleic acids. The particles may be rod shaped or spherical or disc shaped, or comprise any other shape. The particles may also be distinguishable by their shape or size or physical location. The particles may be distinguished through spectroscopy by having a composition containing dyes or fluorochromes in various ratios or concentrations. The particles may also be distinguishable by barcode or holographic images or other imprinted forms of particle coding. Where the particles are magnetic particles, they may be attracted to the surface of the chamber by application of a magnetic field and the magnetic particles may be dispersed from the surface of the chamber by removal of the magnetic field. The magnetic particles are preferably paramagnetic or superparamagnetic.

The term “gel” in this context refers to a semi-rigid solid that is permeable to liquids and gases. As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein specifically incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. Hydrogels can be derived from a single species of monomer or from two or more different monomer species with at least one hydrophilic component. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure® XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

As used herein, the term “reaction chamber” refers to a contained space or vessel designed for conducting chemical, biological, or physical reactions. A reaction chamber may include features such as inlets and outlets for introducing and removing substances, sensors for monitoring reaction conditions, and mechanisms for agitation or mixing. As used herein, the term “inlet” or “inlet port” refers to the location on a flow cell assembly where the reagents and fluids used for methods described herein enters the flow cell. As used herein, the term “outlet” or “outlet port” refers to the location on a flow cell assembly where the reagents and fluids used for methods described herein exits the flow cell after contacting the reaction chamber containing the cell or tissue to be analyzed. In embodiments, the reaction chamber is a part of the flow cell where the cell or tissue is in contact with the fluids (e.g., buffers), polymerases, nucleotides, and reagents used for the methods described herein. In embodiments, the reaction chamber is formed when a first solid support and a second solid support configured to provide a channel are attached together. In embodiments, the reaction chamber is an enclosed (i.e., closed) container containing one or two openings for introducing and removing fluids and reagents.

The discrete regions (i.e., features, wells) of the microplate or flow cell may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “feature” refers a point or area that can be distinguished from other points or areas according to its relative location on a flow cell or microplate. An individual feature can include one or more polynucleotides. For example, a feature can include a single target nucleic acid molecule having a particular sequence or a feature can include several nucleic acid molecules having the same sequence (and/or complementary sequence, thereof). Different molecules that are at different features of a pattern can be differentiated from each other according to the locations of the features in the pattern. Non-limiting examples of features include wells in a substrate, particles (e.g., beads) in or on a substrate, polymers in or on a substrate, projections from a substrate, ridges on a substrate, or channels in a substrate. In embodiments, the one or more features include a reaction chamber and its contents. In embodiments, the one or more features includes a target (e.g., a nucleic acid, protein, or biomarker), a cell, or a tissue sample. In embodiments, the feature is a nucleotide (e.g., a fluorescently labeled nucleotide). In embodiments, the feature is a nucleic acid. In embodiments, the feature is a protein. In embodiments, the feature is a biomolecule.

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and refers to any abnormal biological or aberrant state of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatosis, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5 kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WTI1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or strepavidin to form a avidin-biotin complex or streptavidin-biotin complex.

As used herein, a “probe” (e.g., a first probe, a second probe, a third probe, etc.) refers to an agent that includes (1) a binding moiety capable of specifically binding to a biomolecule of interest and (2) a detectable label to enables the detection of the biomolecule of interest. The compositions and kits described herein utilize a probe to facilitate the serial detection of a biomolecule of interest in a cell or tissue by forming a complex including the probe described herein bound to the biomolecule described herein and detecting the complex. In embodiments, the probe further includes a quenching moiety attached to the probe via a cleavable linker. In embodiments, the probe is a specific binding reagent. In embodiments, the probe includes an antibody, single-chain Fv fragment (scFv), antibody fragment-antigen binding (Fab), affimer, or an aptamer. In embodiments, the probe is specific to a protein of interest. In embodiments, the probe is specific to an oligonucleotide of interest. In embodiments, the probe includes an antibody that is specific to a protein of interest. In embodiments, the probe includes an oligonucleotide that is specific to a nucleic acid of interest.

As used herein, a “complex” refers to a molecular entity formed by binding a probe described herein with a biomolecule described herein, wherein the complex includes a moiety that is capable of specifically binding to a biomolecule of interest (i.e., a biomolecule-specific binding agent) bound to the biomolecule. The compositions and kits described herein utilize a probe to facilitate the serial detection of a biomolecule of interest in a cell or tissue by forming a complex including the probe described herein bound to the biomolecule described herein and detecting the complex.

As used herein, a “specific binding reagent” refers to an agent that binds specifically to a particular biomolecule (e.g., carbohydrate, cell surface receptor, protein of interest, nucleic acid of interest, or lipid molecule of interest). Examples of a specific binding reagent include, but are not limited to, an antibody or target-specific oligonucleotide.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

A “monoclonal antibody” comprises a collection of identical molecules produced by a single B cell lymophocyte clone which are directed against a single antigenic determinant. Monoclonal antibodies can be distinguished from polyclonal antibodies in that monoclonal antibodies must be individually selected whereas polyclonal antibodies are selected in groups of more than one or, in other words, in bulk. Large amounts of monoclonal antibodies can be produced by immortalization of a polyclonal B cell population using hybridoma technology. Each immortalized B cell can divide, presumably indefinitely, and gives rise to a clonal population of cells that each expresses an identical antibody molecule. The individual immortalized B cell clones, the hybridomas, are segregated and cultured separately.

The term “polyclonal antibody” refers to an antibody that is produced from a different B cell lineages within the body. A polyclonal antibody is directed to many different antigenic determinants on the target cell surface and would bind with sufficient density to allow the effector mechanisms of the immune system to work efficiently.

An immunoglobulin (antibody) structural unit are typically tetrameric glycosylated proteins. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

Thus, the term antibody, as used herein, includes antibody fragments either produced by the modification of whole antibodies, or those synthesized de novo either chemically or using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301. As appreciated by one of skill in the art, various antibody fragments can be obtained by a variety of methods, for example, digestion of an intact antibody with an enzyme, such as pepsin; or de novo synthesis.

The term “aptamer” refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their +complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

An “antigen” (Ag) is any substance that reacts specifically with antibodies or T lymphocytes (T cells). In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef; HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the biomolecule-specific binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype O11); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukDE leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gplOO, dipeptidyl peptidase IV (DPPIV), adenosine deaminase-binding protein (ADAbp), cyclophilin b, colorectal associated antigen (CRC)-COI 7-1 A/GA733, carcinoembryonic antigen (CEA) and its immunogenic epitopes CAP-1 and CAP-2, etvβ, am11, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulfites. These lists are not meant to be limiting

An “affimer” is a non-antibody protein that binds to target proteins with affinity in the nanomolar range. It behaves similarly to an antibody by binding tightly to its target molecule. Affimers are recombinant proteins that are typically engineered to mimic molecular recognition characteristics of monoclonal antibodies.

As used herein, the term “immunoassay” refers to a biochemical test that measures the presence or concentration of a macromolecule or a small molecule in a solution involving a reaction between an antibody and an antigen. The molecule detected by the immunoassay is often referred to as an “analyte” and is in many cases a protein, although it may be other kinds of molecules, of different sizes and types. Immunoassays come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. Immunoassays include assays in which the analyte is an antigen, as well as assays in which the analyte is an antibody (e.g., when detecting the presence, absence, or degree of an immune response). In embodiments, an immunoassay includes detecting multiple different analytes from a single sample simultaneously in a common reaction volume.

An “analyte-specific binding agent” is a substance that allows for selective binding to another substance (e.g. an analyte). A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. A binding agent is typically a biological or synthetic molecule that has high affinity for another molecule or macromolecule, through covalent or non-covalent bonding. Examples of a binding agent can include streptavidin, antibody, antigen, enzyme, enzyme cofactor or inhibitor, hormone, or hormone receptor. This binding agent can bind to an analyte (e.g., a protein), often in a heterogeneous population of proteins and other biologics. Thus, under designated immunoassay conditions, the specified binding agents bind to a particular analyte at least two times the background and more typically more than 10 to 100 times background.

As used herein, the term “analyte” refers to a component, substance, or constituent of interest in an analytical procedure whose presence, absence, or amount is desired to be determined or measured. In an immunoassay, for example, the analyte may be a protein, protein fragment, polypeptide, an antibody, antigen expressing antibody or a molecule detectable with an antibody, an antigen, or a ligand. The term “analyte” also refers to detectable components of structured elements such as cells, including all animal and plant cells, and microorganisms, such as fungi, viruses, bacteria including, but not limited to, all gram positive and gram negative bacteria, and protozoa.

In some embodiments, a “sample” includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween®, BSA). Non-limited examples of reservoirs include cartridges, pouches, vials, containers, and Eppendorf® tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

The term “clonotype” is used in accordance with its ordinary meaning in the art and refers to a recombined nucleic acid which encodes an immune receptor or a portion thereof. For example, a clonotype refers to a recombined nucleic acid, usually extracted from a T cell or B cell, but which may also be from a cell-free source, which encodes a T cell receptor (TCR) or B cell receptor (BCR), or a portion thereof. In embodiments, clonotypes may encode all or a portion of a VDJ rearrangement of IgH, a DJ rearrangement of IgH, a VJ rearrangement of IgK, a VJ rearrangement of IgL, a VDJ rearrangement of TCR β, a DJ rearrangement of TCR β, a VJ rearrangement of TCR α, a VJ rearrangement of TCRγ, a VDJ rearrangement of TCR δ, a VD rearrangement of TCR δ, a Kde-V rearrangement, or the like. Clonotypes may also encode translocation breakpoint regions involving immune receptor genes, such as Bcl1-JH or Bcl2-JH. In one aspect, clonotypes have sequences that are sufficiently long to represent or reflect the diversity of the immune molecules that they are derived from consequently, clonotypes may vary widely in length. In some embodiments, clonotypes have lengths in the range of from 25 to 400 nucleotides; in other embodiments, clonotypes have lengths in the range of from 25 to 200 nucleotides.

A “immune repertoire” refers to the collection of T cell receptors and B cell receptors (e.g., immunoglobulin) that constitutes an organism's adaptive immune system.

A “locus” is used in accordance with its ordinary meaning and refers to a location of a gene or other DNA sequence on a chromosome. The Immunoglobulin Heavy (IGH) locus refers to a collection of located on chromosome 14 and is responsible for the production of heavy chain immunoglobulins, composed of several sub-loci, including V, D, J, C and S regions, which are involved in the process of antibody diversity. The IGH locus is responsible for the production of IgM, IgD, IgG, IgE, and IgA. The Immunoglobulin Kappa (IGK) locus refers to a collection of genes located on chromosome 2 and is responsible for the production of kappa light chain immunoglobulins, composed of V, J, and C regions, which are involved in the process of antibody diversity. The IGK locus is responsible for the production of IgM, IgD, IgG, IgE, and IgA. The Immunoglobulin Lambda (IGL) locus refers to a collection of genes located on chromosome 22 and is responsible for the production of lambda light chain immunoglobulins, composed of V, J, and C regions, which are involved in the process of antibody diversity.

By aqueous solution herein is meant a liquid including at least 20 vol % water. In embodiments, aqueous solution includes at least 50%, for example at least 75 vol %, at least 95 vol %, above 98 vol %, or 100 vol % of water as the continuous phase.

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

As used herein, the term “identification oligonucleotide” can also refer to a “barcode” or “index” or “unique molecular identifier (UMI)” and refers to a known nucleic acid sequence which has feature(s) that can be identified. Typically, an identification oligonucleotide is unique to a particular feature in a pool of identification oligonucleotide that differ from one another in sequence, and each of which is associated with a different feature. In embodiments, identification oligonucleotides are about or at least about 5, 6, 7, 8, 9, 10, 15, 20, 25, 30, 40, 50, 75 or more nucleotides in length. In embodiments, identification oligonucleotides are shorter than 20, 15, 10, 9, 8, 7, 6, or 5 nucleotides in length. In embodiments, identification oligonucleotides are 10-50 nucleotides in length, such as 15-40 or 20-30 nucleotides in length. In a pool of different identification oligonucleotides, identification oligonucleotides may have the same or different lengths. In general, identification oligonucleotides are of sufficient length and comprise sequences that are sufficiently different to allow the identification of associated features (e.g., a binding agent or analyte) based on identification oligonucleotides with which they are associated. In embodiments, an identification oligonucleotide can be identified accurately after the mutation, insertion, or deletion of one or more nucleotides in the identification oligonucleotide sequence, such as the mutation, insertion, or deletion of 1, 2, 3, 4, 5, or more nucleotides. In embodiments, each identification oligonucleotide in a plurality of identification oligonucleotides differs from every other identification oligonucleotide in the plurality by at least three nucleotide positions, such as at least 3, 4, 5, 6, 7, 8, 9, 10, or more nucleotide positions.

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent.

As used herein, the term “detectable moiety” or “detectable agent” or “detectable label” can also refer to a “label” or “labels” and generally refer to molecules that can directly or indirectly produce or result in a detectable signal either by themselves or upon interaction with another molecule. Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e. cyanine 3 or Cy®3). In embodiments, the cyanine moiety has 5 methine structures (i.e. cyanine 5 or Cy®5). In embodiments, the cyanine moiety has 7 methine structures (i.e., cyanine 7 or Cy®7). In embodiments, a detectable moiety is a moiety (e.g., monovalent form) of a detectable agent.

The terms “fluorophore,” “fluorescent agent,” “fluorescent dye,” or “fluorescent dye moiety” are used interchangeably and refer to a substance, compound, agent, or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon Green®, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5™, DRAQ7™, or CyTRAK Orange™), pyrene derivatives (e.g., cascade blue and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF® dye, DRAQ™, CyTRAK™, BODIPY®, Alexa Fluor®, DyLight®, Atto®, Tracy, FluoProbes®, Abberior® Dyes, DY dyes, MegaStokes Dyes, Sulfo Cy®, Seta dyes, SeTau dyes, Square Dyes, Quasar® dyes, Cal Fluor™ dyes, SureLight® Dyes, PerCP, Phycobilisomes, APC, APCXL™, RPE, and/or BPE. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

The term “rhodamine” as is used in accordance with its ordinary meaning in the art and refers to a detectable moiety including a xanthene backbone. Structurally, rhodamine is a family of related polycyclic dyes with a xanthene core, i.e.,

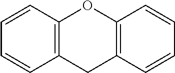 [SMILES: c1ccc2c(c1)Cc1ccccc1O2]

(xanthene). Generally speaking, functional groups on the conjugated moiety of the xanthene core have the ability to fine tune the fluorescent colors. Non-limiting examples of rhodamine dyes include Rhodamine B, Rhodamine 6G, Rhodamine 123, and Rhodamine WH. Rhodamine derivatives have also been disclosed, such as in PCT Int. Appl, WO 2009108905; U.S. Pat. Nos. 5,728,529; 5,686,261; and by Kim et al. (Journal of Physical Chemistry A (2006), 110(1), 20-27)).

The term “sulforhodamine 101 moiety” is used in accordance with its ordinary meaning in the art and refers to detectable moiety containing xanthene backbone with the following structure:

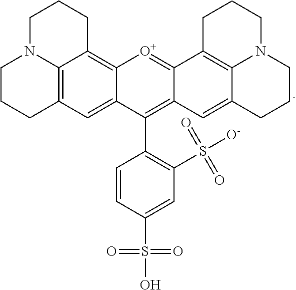 [SMILES: O=S(=O)([O-])c1cc(S(=O)(=O)O)ccc1-c1c2cc3c4c(c2[o+]c2c5c6c(cc12)CCCN6CCC5)CCCN4CCC3]

The sulforhodamine 101 moiety is a red fluorescent dye and commonly used for astrocyte identification. An example of a commercially available dye with a sulforhodamine 101 moiety is Texas Red.

The term “fluorescein moiety” is used in accordance with its ordinary meaning in the art and refers to a detectable moiety containing xanthene backbone having the following structural formula:

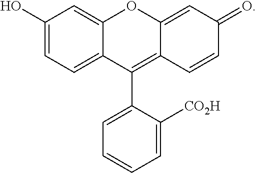 [SMILES: O=C(O)c1ccccc1-c1c2ccc(=O)cc-2oc2cc(O)ccc12]

Dyes with fluorescein moieties are commonly used as fluorescent probes in life sciences and medical applications due to their hydrophilicity, high absorptivity, and high quantum yield. Examples of detectable agents containing fluorescein moieties include fluorescein reactive dyes, which are fluorescein dyes derivatized with different bioconjugation moieties (e.g., maleimide, NHS, or isothiocyanate moieties).

The term “fluorescein isothiocyanate moiety” is used in accordance with its ordinary meaning in the art and refers to a detectable moiety containing xanthene backbone derived from a fluorescein moiety. Detectable agents harboring a fluorescein isothiocyanate moiety has the following structural formula:

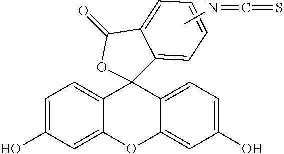

and are primarily used to label primary amines of a biomolecule. Commercially available forms of detectable agents with a fluorescein isothiocyanate moiety include fluorescein 5-isothiocyanate (5-FITC), fluorescein 6-isothiocyanate (6-FITC), or a mixture of the two isomers.

The term “cyanine” or “cyanine moiety” is used in accordance with its ordinary meaning in the art and refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy®5). In embodiments, the cyanine moiety has 7 methine structures (i.e., cyanine 7 or Cy®7). Cyanine dyes refer to a family of dyes in which the chromophoric system includes conjugated double bonds connecting two end groups consisting of an electron acceptor and an electron donor. There are three types of cyanine dyes: (1) closed chain cyanines of the general structure:

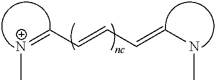

(2) hemicyanines of the general structure:

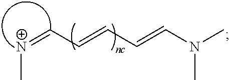

and (3) open chain cyanines of the general structure:

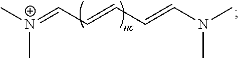

where nc is an integer from 1 to 9.

The term “indocyanine green moiety” is used in accordance with its ordinary meaning in the art and refers to a detectable moiety from the cyanine family of dyes. Specifically, an indocyanine green moiety consists of a cyanine 7 dye moiety of the following structure:

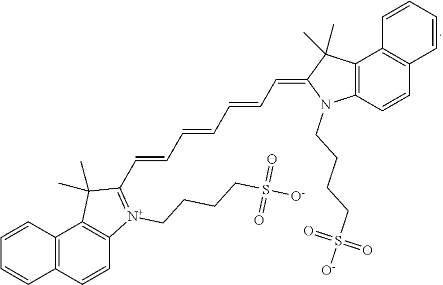 [SMILES: CC1(C)C(/C=C/C=C/C=C/C=C2\N(CCCCS(=O)(=O)[O-])c3ccc4ccccc4c3C2(C)C)=[N+](CCCCS(=O)(=O)[O-])c2ccc3ccccc3c21]

The term “triarylmethane” is used in accordance with its ordinary meaning in the art and refers to a detectable moiety containing a triarylmethane backbone. A triarylmethane dye is derived from a triaryl methane compound and is used for colorimetric assays, analytical chemistry, and are used to color fabrics and plastics, as well as in inks and paints. Examples of triaryl methane dyes include Malachite Green, Crystal Violet, Methyl Violet, Methylene Blue, and Phenol Red.

The term “coumarin moiety” used in accordance with its ordinary meaning in the art and refers to a detectable moiety containing a benzene and α-pyrone rings of the general structure:

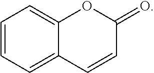 [SMILES: O=c1ccc2ccccc2o1]

Dyes with a coumarin moiety are typically excited with electromagnetic radiation from the UV range and emit between 400-470 nm. Examples of commercially available dyes derived containing a coumarin moiety include DiFMUP (6,8-difluoro-4-methylumbelliferyl phosphate) and AMC (7-amino-4-methylcoumarin).

As used herein, the term “photodamage mitigating agent” refers to a composition that may prevent photodamage of one or more reagents, or it may mitigate the impact that a photodamaged reagent may have on a particular, limited reagent in the reaction of interest. By way of example, an agent that blocks a detrimental interaction between a photodamaged fluorescent compound and a critical enzyme component would still be referred to as a photodamage mitigating agent, regardless of the fact that it did not prevent the initial photodamage to the fluorescent reagent. In particular, photodamage mitigating agents are provided in the context of the analytical reaction to reduce the level of photodamage (and/or increase the photodamage threshold period), that would otherwise have occurred but for the presence of the photodamage mitigating agent. In general, the photodamage mitigating agents are present in the reaction mixture at levels sufficient to provide beneficial impact, e.g., reduced photodamage and/or extension of the photodamage threshold period, but are not present at such levels as to interfere with the reaction of interest, e.g., the sequencing reaction. Non-limiting examples of a photodamage mitigating agent include ascorbic acid, dithiothreitol (DTT), mercaptoethylamine (MEA), P-mercaptoethanol (BME), N-propyl gallate, p-phenylenediamene (PPD), hydroquinone, sodium azide (NaN₃), diazobicyclooctane (DABCO), cyclooctatetraene (COT), Trolox and its derivatives, butylated hydroxytoluene (BHT), ergothioneine, methionine, cysteine, beta-dimethyl cysteine, histidine, tryptophan, mercaptopropionylglycine, MESNA, glutathione, N-acetyl cysteine, captopril, lycopene, gamma-carotene, astazanthin, canthazanthin, alpha-carotene, beta-carotene, gamma-carotene, bixin, zeaxanthin, lutein, bilirubin, biliverdin, tocopherols, polyene dialdehydes, 32 melatonin, octocopheryl succinate and its analogs, pyridoxinel and its derivatives, hydrazine, sodium sulfite, and hydroxylamine. In embodiments, the photodamage mitigating agent is sodium pyruvate, N,N′-dimethylthiourea, mannitol, DMSO, carboxy-PTIO, 6-hydroxy-2,5,7,8-tetramethylchroman-2-carboxylic acid, alpha-tocopherol, 2-phenyl-1,2,benzisoselenazol-3(2H)-one, uric acid, sodium azide, or manganese(III)-tetrakis(4-benzoic acid) porphyrin, 4,5-dihydroxybenzene-1,3-disulfonate. In embodiments, the photodamage mitigating agent is 3-carboxy-proxyl, N-propyl gallate, ascorbic acid, methyl viologen, Trolox, or Trolox-quinone.

As used herein, the term “photodamage” refers to any direct or indirect impact of illumination on one or more reagents in a desired reaction, such that it results in a negative impact upon that reaction. As such, photodamage would include a direct photoinduced change in a given reagent so as to reduce the reactivity of that reagent in the desired reaction, e.g., photobleaching of a fluorescent molecule, or otherwise reduce its usefulness in such reaction, e.g., by making the reagent less specific in the given reaction. Likewise, photodamage would include negative changes in a reagent that are caused by interaction of that reagent with a product of another photo-induced reaction, e.g., the generation of singlet oxygen during a fluorescence excitation event, which singlet oxygen may damage organic or other reagents, e.g., proteins.

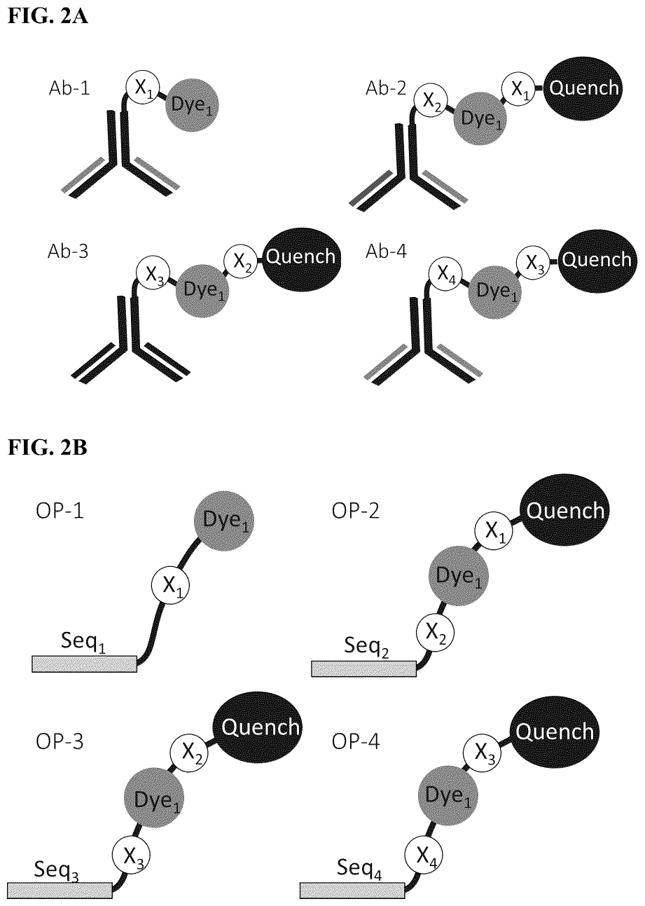

The term “quenching moiety” refers to a monovalent compound capable of reducing the fluorescence intensity of an excited fluorophore. For example, quenching compounds are capable of absorbing energy from a fluorophore (such as a fluorescent dye) and re-emitting much of that energy as either heat in the case of dark quenchers (e.g., Dabcyl) or visible light in the case of fluorescent quenchers (e.g., TAMRA™). Common quenching moieties provided by Life Technologies include QSY™ 7 QSY™ 9, and QSY™ 21 which are capable of broad absorption in the visible-light spectrum, with an absorption maximum near 560 nm for both the QSY™ 7 and QSY™ 9 quenchers and near 660 nm for the QSY™ 21 quencher. The QSY quenching moieties are suitable for quenching blue-fluorescent coumarins, green- or orange-fluorescent dyes, and red-fluorescent Texas Red and Alexa Fluor® 594 conjugates. In embodiments, the quenching moiety absorbs energy from an excited fluorophore, but which does not release fluorescent energy itself. In embodiments, the quenching moiety can attenuate at least partly (i.e., by at least 10%) the light emitted by a fluorescent group. This attenuation is referred to herein as “quenching”. Hence, illumination of a “fluorescent moiety” in the presence of the “quenching moiety” leads to an emission signal that is less intense than expected, or even completely absent. The quencher useful herein can itself be fluorescent, but absorb the energy emitted by the other fluorescent moiety on the dual-labeled nucleotide. In this instance, commonly referred to as Fluorescence Resonance Energy Transfer, or FRET, illumination of the “fluorescent moiety” with light within the excitation spectrum of that moiety will result in non-radiative transfer from that fluorescent moiety to the “quencher moiety,” which then emits at a different wavelength than the fluorescent moiety attached to the nucleobase. In this situation, the “quencher” emits light but, due to its attenuation of the emission of the “fluorescent moiety,” is still considered a “quencher moiety.” In embodiments, the quenching moiety is attached to a detectable label via a first cleavable linker, wherein the detectable label is attached to a probe via a second linker (see, e.g., Ab-2 shown in FIG. 2A or OP-2 shown in FIG. 2B), and cleaving the first cleavable linker shown as X₁ in Ab-2 of FIG. 2A or OP-2 in FIG. 2B enables detection of the detectable label (i.e., Dye₁).

As used herein, the term “FRET pair of detectable moieties” refers to a donor molecule (e.g., first detectable moiety) and an acceptor molecule (e.g., second detectable moiety) capable of undergoing fluorescence resonance energy transfer (FRET). In a FRET pair, a first detectable moiety is excited with an excitation wavelength and non-radiatively transfers the energy to a second detectable moiety, wherein the efficiency of the energy transfer correlates to the separation between the pair of detectable moieties. Changes in the efficiency of FRET are correlated to changes in the separation between the detectable moieties, which may be quantified by measuring the absorbance spectra of a FRET pair. The FRET donor molecule initially absorbs energy (and is thus excited) and then transfers energy, by way of emission, to the FRET acceptor molecule (resulting in excitation of the FRET acceptor molecule). The resonance energy transfer can occur over distances greater than inter-atomic distances, and without conversion to thermal energy nor any molecular collision. The FRET donor or the FRET acceptor can be selected based on a variety of factors such as stability, excitation, and emission wavelengths as well as signal intensity. For example, the FRET acceptor is generally selected such that it is capable of emitting light when excited by light of the wavelength emitted by the FRET donor. The FRET pair could consist of a nonfluorescent FRET acceptor, where following absorption of light from the FRET donor, the nonfluorescent FRET acceptor dissipates the energy as heat (see, e.g., Mechanisms and Dynamics of Fluorescence Quenching. (2006). Principles of Fluorescence Spectroscopy, 331-351). It is understood that FRET includes Time-Resolved FRET (or TR-FRET), which combines the use of long-lived fluorophores and time-resolved detection (a delay between excitation and emission detection) to minimize fluorescent interference due to any inherent fluorescence of, e.g., target molecules or target-selective binding agents (see, e.g., Klostermeier et al. (2001-2002) Biopolymers 61(3):159-79). In some embodiments, the first member of the FRET pair is a FRET donor and the second member of the FRET pair is a FRET acceptor. In some embodiments, the second member of the FRET pair is a FRET donor and the first member of the FRET pair is a FRET acceptor. Exemplary examples of FRET pairs include, but are not limited to, fluorescein/rhodamine, Cy®3/Cy®5, lanthanide/phycobiliprotein, lanthanide/Cy®5, TMR-ATTO®647N, Cy®3-ATTO®647N, and TMR-Cy®5 (See Arai, Y., & Nagai, T. (2013). Extensive use of FRET in biological imaging. Microscopy, 62(4), 419-428; Di Fiori, N., & Meller, A. (2010). The Effect of Dye-Dye Interactions on the Spatial Resolution of Single-Molecule FRET Measurements in Nucleic Acids. Biophysical Journal, 98(10), 2265-2272; U.S. Pat. No. 11,186,870; and PCT Publication No. WO09/10558). Additional examples of useful FRET labels include, e.g., those described in U.S. Pat. Nos. 5,654,419, 5,688,648, 5,853,992, 5,863,727, 5,945,526, 6,008,373, 6,150,107, 6,177,249, 6,335,440, 6,348,596, 6,479,303, 6,545,164, 6,849,745, 6,696,255, and 6,908,769 and Published U.S. Patent Application Nos. 2002/0168641, 2003/0143594, and 2004/0076979.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable, a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO™ Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method of detecting multiple targets. In embodiments, the method includes: (i) contacting a cell or tissue with a probe including a detectable label and a quenching moiety, wherein the detectable label is attached to the probe via a cleavable linker; (ii) binding the probe to a target of the cell or tissue; (iii) removing the quenching moiety and detecting the probe bound to a target; (iv) cleaving the cleavable linker; and repeating steps (i) to (iii) to detect multiple targets. In embodiments, the target is a biomolecule. In embodiments, the biomolecule is a lipid, carbohydrate, peptide, protein, or antigen binding fragment. In embodiments, the biomolecule is a lipid. In embodiments, the biomolecule is a carbohydrate. In embodiments, the biomolecule is a peptide. In embodiments, the biomolecule is a protein. In embodiments, the biomolecule is an antigen binding fragment. In embodiments, the biomolecule is an oligonucleotide. In embodiments, the target is a nucleic acid. In embodiments, the target is a non-nucleic acid target. Non-nucleic acid targets include, but are not limited to, lipids, carbohydrates, peptides, proteins, glycoproteins, lipoproteins, phosphoproteins, acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In embodiments, the target is inside a cell or on a cell surface, such as a transmembrane analyte or one that is attached to the cell membrane. In embodiments, the target is an organelle (e.g., nuclei or mitochondria).

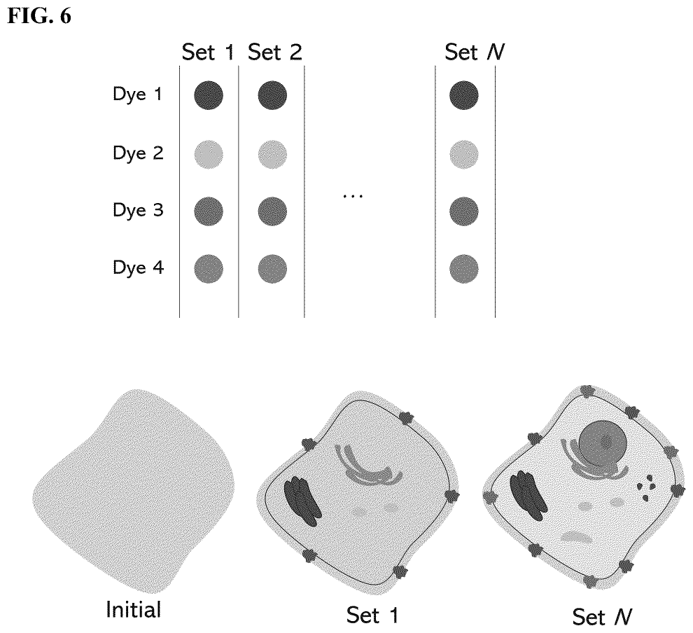

In embodiments, the method includes: contacting a cell or tissue with a first probe set, wherein the first probe set includes a plurality of probes, wherein each probe is attached to a first detectable label; binding each probe to a different target of the cell or tissue; serially detecting each probe bound to a different target; contacting the cell or tissue with a second probe set, wherein the second probe set includes a plurality of probes, wherein each probe is attached to a second detectable label; binding each probe to a different target of the cell or tissue; and serially detecting each probe bound to a different target. In embodiments, the first probe set include 2 or more spectrally distinct fluorescent moieties, and the second set includes the same spectrally distinct fluorescent moieties as the first probe set (e.g., the first set includes Alexa Fluor® 532 and Alexa Fluor® 680, and the second set also includes Alexa Fluor® 532 and Alexa Fluor® 680; see, e.g., FIG. 6).

In embodiments, the method includes: contacting a cell or tissue with a probe set, wherein the probe set includes a first probe including a first detectable label, wherein the first detectable label is attached to the first probe via a first cleavable linker; a second probe including the first detectable label and a quenching moiety, wherein the first detectable label is attached to the second probe via a covalent linker; and the quenching moiety is attached to the second probe via the first cleavable linker; binding the first probe to a first target of the cell or tissue and binding the second probe to a second target of the cell or tissue; detecting the first detectable label attached to the first probe; followed by cleaving the first cleavable linker; detecting the first detectable label attached to the second probe, thereby detecting multiple targets. For example, detecting the first probe in Set 1 depicted in FIG. 6 enables the detection of the Golgi apparatus, endoplasmic reticulum, actin, lysosomes, and specific cell surface receptors of a cell. Following cleavage and removal of the fluorophores from the first probe set, the second set of probes are incubated with the sample cell. The second set can then illuminate the nucleus, nucleoli, mitochondria, nuclear envelop, cell surface receptors, and plasma membrane. The sequential addition of cell paints can continue for N cycles providing additional information about the cell. The resulting images may be computationally processed and overlaid to provide a composite image of the cell and/or tissue.

In an aspect is provided a method of detecting multiple biomolecules, the method including: (a) contacting a cell or tissue with a first probe and a second probe, thereby forming a first complex and a second complex, wherein the first complex includes the first probe bound to a first biomolecule, wherein the first probe includes a first detectable label attached to the first probe via a first linker; the second complex includes the second probe bound to a second biomolecule, wherein the second probe includes a second detectable label and a quenching moiety, wherein the second detectable label is attached to the second probe via a second linker; and the quenching moiety is attached to the second probe via a first cleavable linker; (b) detecting the first complex; cleaving the first cleavable linker, thereby separating the quenching moiety from the second complex; and (c) detecting the second complex. In embodiments, the first linker includes a cleavable site (i.e., the first linker is a cleavable linker).

In another aspect is provided a method of detecting multiple biomolecules, the method including: (a) contacting a cell or tissue with a first probe and a second probe, thereby forming a first complex and a second complex, wherein the first complex includes the first probe bound to a first biomolecule, wherein the first probe includes a first detectable label attached to the first probe via a first cleavable linker; the second complex includes the second probe bound to a second biomolecule, wherein the second probe includes a second detectable label, wherein the second detectable label is attached to the second probe via a second cleavable linker; (b) detecting the first complex and cleaving the first cleavable linker, thereby separating the first detectable label from the first complex; and (c) detecting the second complex and cleaving the second cleavable linker, thereby separating the second detectable label from the second complex. In embodiments, the method further includes contacting the cell or tissue with a stain, wherein the stain binds to a third biomolecule. A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the first fluorescent dye. In embodiments, detecting includes directing an excitation light to the cell or tissue and detecting an emission light from the second fluorescent dye. In embodiments, detecting includes directing an excitation light to the cell or tissue and detecting an emission light from the stain. In embodiments, detecting includes directing an excitation light to the cell or tissue and detecting an emission light from the first fluorescent dye, the second fluorescent dye, and the stain.

In embodiments, the method includes repeating steps (a) and (b) for a third biomolecule. In embodiments, the method includes repeating steps (a), (b), and (c) for a third biomolecule. In embodiments, the method includes repeating steps (a), (b), and (c) for two or more cycles to detect a plurality of biomolecules. In embodiments, the third biomolecule is a nucleic acid molecule, a protein, or a carbohydrate. In embodiments, the third biomolecule is an organelle.

In embodiments, the first detectable label is a fluorescent dye and the second detectable label is a fluorescent dye. In embodiments, the cleavable linker is cleaved thereby forming a cleaved complex. Following cleavage of the cleavable linker, the complex may be referred to as a “cleaved complex.”

In embodiments, the first biomolecule and the second biomolecule each independently a different organelle. In embodiments, the first biomolecule is a mitochondria, a plasma membrane, a Golgi apparatus, an endosome, a peroxisome, a lysosome, a nucleus, a nuclear envelop, or a nucleolus. In embodiments, the second biomolecule is a mitochondria, a plasma membrane, a golgi apparatus, an endosome, a peroxisome, a lysosome, a nucleus, a nuclear envelop, or a nucleolus. In embodiments, the first biomolecule is actin, plasma membrane, mitochondria, endoplasmic reticulum, Golgi apparatus, lysosome, phosphatidylserine, transferrin receptor, or a carbohydrate. In embodiments, the second biomolecule is actin, plasma membrane, mitochondria, endoplasmic reticulum, Golgi apparatus, lysosome, phosphatidylserine, transferrin receptor, or a carbohydrate. In embodiments, the first biomolecule is a nucleic acid molecule, a protein, or a carbohydrate. In embodiments, the second biomolecule is a nucleic acid molecule, a protein, or a carbohydrate.

In embodiments, prior to contacting a cell or tissue with a first probe and a second probe, the method further includes immobilizing a cell or tissue section onto a solid support (e.g., a flow cell or well in a microplate). In embodiments, prior to contacting a cell or tissue with a first probe and a second probe, the method further includes immobilizing a cell or tissue section onto a flow cell. In embodiments, prior to contacting a cell or tissue with a first probe and a second probe, the method further includes immobilizing a cell or tissue section into a well of a microplate. In embodiments, prior to contacting a cell or tissue with a probe including a detectable label and a quenching moiety, the method further includes immobilizing a cell or tissue section onto a flow cell. In embodiments, prior to contacting a cell or tissue with a probe including a detectable label and a quenching moiety, the method further includes immobilizing a cell or tissue section into a well of a microplate. In embodiments, the solid support includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate.

In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

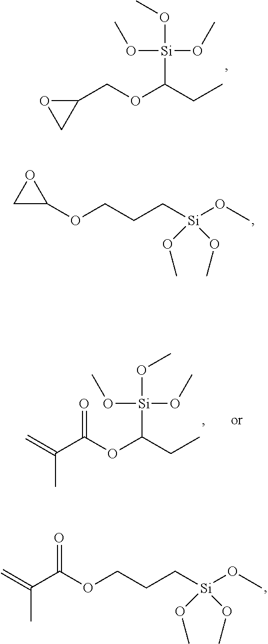

or a copolymer thereof. In embodiments, the polymer layer is an organically-modified ceramic polymer. In embodiments, the polymer includes polymerized monomers of alkoxysilyl polymers, such as

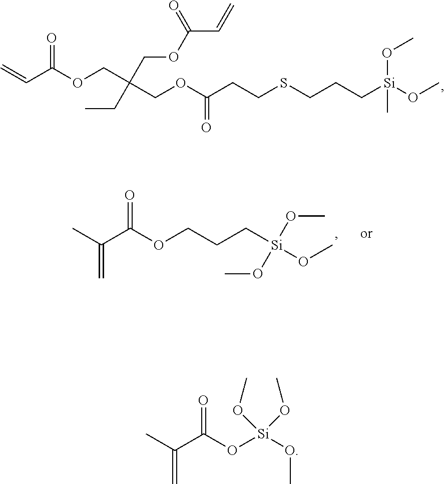

In embodiments, the solid support includes polymerized units of

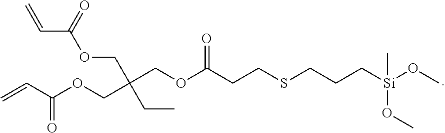 [SMILES: C=CC(=O)OCC(CC)(COC(=O)C=C)COC(=O)CCSCCC[Si](C)(OC)OC]

In embodiments, the solid support includes polymerized units of

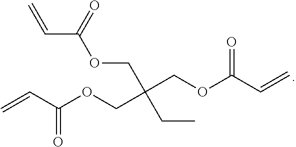 [SMILES: C=CC(=O)OCC(CC)(COC(=O)C=C)COC(=O)C=C]

In embodiments, the solid support includes polymerized unites of

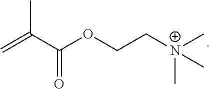

In embodiments, the polymer layer includes one or more ceramic particles, (e.g., silicates, aluminates, and titanates). In embodiments, the polymer layer includes tantalum(V) oxide, titanium dioxide, zinc oxide, and/or iron oxide.

In embodiments, the method includes immobilizing a plurality of tissue sections to the solid support (e.g., a flow cell), wherein a tissue in a plurality of tissue sections includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 μm sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the first solid support as described herein (e.g., inverting the punch device); pushing the sample portion out of the punch device using a piston, so that all or a portion thereof of the sample portion is positioned on the first solid support as described herein. In embodiments, the method further includes cutting a sample portion from the biological sample using two or more punch devices such that each punch device contains a different the sample portion; mounting each punch device containing the sample portion onto the first solid support as described herein; pushing the sample portions out of the punch devices using one or more pistons so that the sample portions are positioned onto the solid support as described herein (e.g., a flow cell described herein).

In embodiments, the probe includes a binding moiety capable of binding the biomolecule. In embodiments, the binding moiety includes an oligonucleotide including a target hybridization sequence. For example, as illustrated in FIG. 2B, the oligonucleotide probes (OP) include a target hybridization sequence. In embodiments, the oligonucleotide probe is about 50 to about 500 nucleotides in length. In embodiments, the oligonucleotide probe is about 50 to about 300 nucleotides in length. In embodiments, the oligonucleotide probe is about 80 to about 300 nucleotides in length. In embodiments, the oligonucleotide probe is about 50 to about 150 nucleotides in length. In embodiments, the oligonucleotide probe is about or more than about 40, 50, 75, 100, 125, 150, 175, 200, 250, 300, 350, 400, or 500 nucleotides in length. In embodiments, the oligonucleotide probe is less than about 50, 75, 100, 125, 150, 175, 200, 250, 300, 350, 400, or 500 nucleotides in length. In embodiments, the target hybridization sequence includes 5 to 25 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 15 to 30 nucleotides in length. In embodiments, the target hybridization sequence (e.g., the first and/or second target hybridization sequence) is greater than 30 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is about 5, about 10, about 15, about 20, about 25, about 30, or about 35 nucleotides in length. In embodiments, the target hybridization sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the target hybridization sequence of each oligonucleotide primer is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence includes about 45% to 65% GC content (i.e., the percentage of nucleobases that are either guanine or cytosine). In embodiments, the target hybridization sequence does not include 4 or more guanine or cytosine nucleobases.

In embodiments, the target hybridization sequence of each probe (e.g., each probe of a plurality of probes) is complementary to different portions of the same target polynucleotide. In embodiments, the target hybridization sequence of each probe (e.g., each probe of a plurality of probes) is complementary to different portions of different target polynucleotides. In embodiments, the target hybridization sequence of each probe is complementary to portions of the same target polynucleotide that are separated by about 10 to about 500 nucleotides. In embodiments, the target hybridization sequence of each probe are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the target hybridization sequence of each probe is complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides. In embodiments, the target hybridization sequence includes one or more modified nucleotide(s). In embodiments, the modified nucleotide includes one or more locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), phosphorothioate nucleic acids, Zip nucleic acids (ZNAs), or combinations thereof. In embodiments, the first target hybridization sequence includes one or more locked nucleic acids (LNAs), Zip nucleic acids (ZNAs), 2-amino-deoxyadenosine (2-amino-dA), trimethoxystilbene-functionalized oligonucleotides (TFOs), Pyrene-functionalized oligonucleotides (PFOs), peptide nucleic acids (PNAs), or aminoethyl-phenoxazine-dC (AP-dC) nucleic acids. In embodiments, the target hybridization includes from 5′ to 3′ a plurality (e.g., 2 to 5) of phosphorothioate nucleic acids, followed by a plurality of synthetic nucleotides (e.g., LNAs), and subsequently followed by a plurality (e.g., 2 to 5) of canonical nucleobases. In some embodiments, the target hybridization sequence includes a plurality of canonical nucleobases, wherein the canonical nucleobases terminate (i.e., at the 3′ end) with a deoxyuracil nucleobase (dU).

In embodiments, the target hybridization sequence includes a plurality of LNAs interspersed throughout the polynucleotide. In embodiments, the target hybridization sequence includes a plurality of consecutive LNAs (e.g., 2 to 5 LNAs, 5 to 7 LNAs, or 7 to 10 LNAs) throughout the target hybridization sequence and/or probe sequence. In embodiments, the target hybridization sequence includes a plurality of consecutive LNAs (e.g., 2 to 5 LNAs, 5 to 7 LNAs, or 7 to 10 LNAs) throughout the target hybridization sequence. In embodiments, the target hybridization sequence includes a plurality of consecutive LNAs (e.g., 2 to 5 LNAs, 5 to 7 LNAs, or 7 to 10 LNAs) throughout the probe sequence. In embodiments, the entire composition of the target hybridization sequence includes less than 70%, less than 60%, less than 50%, less than 40%, less than 30%, less than 20%, less than 10%, or less than 5% of LNAs. In embodiments, the entire composition of the target hybridization sequence includes up to about 70%, up to about 60%, up to about 50%, up to about 40%, up to about 30%, up to about 20%, up to about 10%, or up to about 5% of LNAs. In embodiments, the entire composition of the target hybridization sequence includes more than 60%, more than 50%, more than 40%, more than 30%, more than 20%, more than 10%, or more than 5% of LNAs. In embodiments, the entire composition of the target hybridization sequence includes about 5% to about 10%, about 10% to about 20%, about 20% to about 30%, about 30% to about 40%, about 40% to about 50%, about 50% to about 60%, or about 60% to about 70% of LNAs. In embodiments, the entire composition of the target hybridization sequence includes about 5%, about 10%, about 15%, about 20%, about 25%, about 30%, about 35%, about 40%, about 45%, about 50%, about 55%, about 60%, about 65%, or about 70% of LNAs. In embodiments, the entire composition of the target hybridization sequence includes about 30%, about 35%, about 40%, about 45%, about 50%, about 55%, about 60%, about 65%, about 70%, about 75%, about 80%, about 85%, about 90%, or about 95% of canonical dNTPs. In embodiments, the entire composition of the target hybridization sequence includes less than 95%, less than 90%, less than 80%, less than 70%, less than 60%, less than 50%, less than 40%, or less than 30% of canonical dNTPs. In embodiments, the entire composition of the target hybridization sequence includes up to about 95%, up to about 90%, up to about 80%, up to about 70%, up to about 60%, up to about 50%, up to about 40%, or up to about 30% of canonical dNTPs. In embodiments, the entire composition of the target hybridization sequence includes more than 90%, more than 80%, more than 70%, more than 60%, more than 50%, more than 40%, or more than 30% of canonical dNTPs.

In embodiments, the probe includes a binding moiety capable of binding the biomolecule, wherein the binding moiety is a specific binding reagent. In embodiments, the binding moiety includes an antibody, single-chain Fv fragment (scFv), antibody fragment-antigen binding (Fab), affimer, or an aptamer. In embodiments, the binding moiety is an antibody. In embodiments, the binding moiety is a single-chain Fv fragment (scFv). In embodiments, the binding moiety is an antibody fragment-antigen binding (Fab). In embodiments, the binding moiety is an affimer. In embodiments, the binding moiety is an aptamer. In embodiments, the binding moiety is a protein, a peptide, an antibody (or an epitope binding fragment thereof), a lipophilic moiety (e.g., cholesterol), a cell surface receptor binding molecule, a receptor ligand, a small molecule, a bi-specific antibody, a bispecific T-cell engager, a T-cell receptor engager, a B-cell receptor engager, a pro-body, an aptamer, a monobody, an affimer, or a darpin.

In embodiments, the analyte or biomolecule is detected from a sample. A sample (e.g., a sample including an analyte) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may comprise cells or tissues that are normal, healthy, diseased (e.g., infected), and/or cancerous (e.g., cancer cells). A sample obtained from a subject may comprise cells or cellular material (e.g., nucleic acids) of multiple organisms (e.g., virus nucleic acid, fetal nucleic acid, bacterial nucleic acid, parasite nucleic acid).

In embodiments, the target polynucleotide is in a cell or tissue. In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell (e.g., a bacterial cell or bacterial spore), a fungal cell (e.g., a fungal spore), a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is a prokaryotic cell. In embodiments, the cell is a bacterial cell. In embodiments, the bacterial cell is a Bacteroides, Clostridium, Faecalibacterium, Eubacterium, Ruminococcus, Peptococcus, Peptostreptococcus, or Bifidobacterium cell. In embodiments, the bacterial cell is a Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp., or Peptococcus sp. cell. In embodiments, the cell is a fungal cell. In embodiments, the fungal cell is a Candida, Saccharomyces, Aspergillus, Penicillium, Rhodotorula, Trametes, Pleospora, Sclerotinia, Bullera, or a Galactomyces cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is bound to a known antigen. In embodiments, the cell includes known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface. In embodiments, the surface includes a patterned surface (e.g., suitable for immobilization of a plurality of cells in an ordered pattern. The discrete regions of the ordered pattern may have defined locations in a regular array, which may correspond to a rectilinear pattern, circular pattern, hexagonal pattern, or the like. These discrete regions are separated by interstitial regions. As used herein, the term “interstitial region” refers to an area in a substrate or on a surface that separates other areas of the substrate or surface. In embodiments, a plurality of cells are immobilized on a patterned surface that have a mean or median separation from one another of about 10-20 μm. In embodiments, a plurality of cells are immobilized on a patterned surface that have a mean or median separation from one another of about 10-20; 10-50; or 100 μm. In embodiments, a plurality of cells are arrayed on a substrate. In embodiments, a plurality of cells are immobilized in a 96-well microplate having a mean or median well-to-well spacing of about 8 mm to about 12 mm (e.g., about 9 mm). In embodiments, a plurality of cells are immobilized in a 384-well microplate having a mean or median well-to-well spacing of about 3 mm to about 6 mm (e.g., about 4.5 mm).

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm. In embodiments, the thickness of the tissue section is about 1 μm. In embodiments, the thickness of the tissue section is about 2 μm. In embodiments, the thickness of the tissue section is about 3 μm. In embodiments, the thickness of the tissue section is about 4 μm. In embodiments, the thickness of the tissue section is about 5 μm. In embodiments, the thickness of the tissue section is about 6 μm. In embodiments, the thickness of the tissue section is about 7 μm. In embodiments, the thickness of the tissue section is about 8 μm. In embodiments, the thickness of the tissue section is about 9 μm. In embodiments, the thickness of the tissue section is about 10 μm. In embodiments, the thickness of the tissue section is about 11 μm. In embodiments, the thickness of the tissue section is about 12 μm. In embodiments, the thickness of the tissue section is about 13 μm. In embodiments, the thickness of the tissue section is about 14 μm. In embodiments, the thickness of the tissue section is about 15 μm. In embodiments, the thickness of the tissue section is less than about 10 μm. In embodiments, the thickness of the tissue section is less about 6 μm, 7 μm, 8 μm, 9 μm or 10 μm. In embodiments, the cell or tissue is obtained via Laser capture microdissection (LCM). LCM is a method for isolating in micrometer-scale tissue or even single cells while retaining spatial information to link histology with molecular measurements. In LCM, a region of interest in the tissue section is isolated through laser cutting.

In embodiments, the tissue section includes a tissue or a cell. Biological tissue samples suitable for use with the methods and systems described herein generally include any type of tissue samples collected from living or dead subjects, such as, for example, tumor tissue and autopsy samples. Tissue samples may be collected and processed using the methods and systems described herein and subjected to microscopic analysis immediately following processing, or may be preserved and subjected to microscopic analysis at a future time, e.g., after storage for an extended period of time. In some embodiments, the methods described herein may be used to preserve tissue samples in a stable, accessible and fully intact form for future analysis. For example, tissue samples, such as, e.g., human tumor tissue samples, may be processed as described herein and cleared to remove a plurality of cellular components, such as, e.g., lipids, and then stored for future analysis. In some embodiments, the methods and systems described herein may be used to analyze a fresh tissue section. In some embodiments, the methods and systems described herein may be used to analyze a previously-preserved (e.g., previously fixed) or stored tissue section (e.g., tissue sample). For example, in some embodiments a previously-preserved tissue sample that has not been subjected to a sample preparation process described herein may be processed and analyzed as described herein. In particular methods, a tissue sample is frozen prior to being processed as described herein.

In certain embodiments, tissue sections are tumor tissue samples. Tumor samples may contain only tumor cells, or they may contain both tumor cells and non-tumor cells. In particular embodiments, a tissue section includes only non-tumor cells. In particular embodiments, the tumor is a solid tumor. In particular embodiments, the tissue section is obtained from or includes an adrenal cortical cancer, anal cancer, aplastic anemia, bile duct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal or squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment, is a tissue section obtained from a subject diagnosed with or suspected of having any of these tumors or cancers.

The methods of the invention can be used to characterize a cancer or metastasis thereof, including without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryoides, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer under analysis may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, brain cancer, kidney cancer, ovarian cancer, stomach cancer, skin cancer, bone cancer, gastric cancer, pancreatic cancer, glioma, glioblastoma, hepatocellular carcinoma, papillary renal carcinoma, head and neck squamous cell carcinoma, leukemia, lymphoma, myeloma, or a solid tumor.

Tissue sections may be obtained from a subject by any means known and available in the art. In particular embodiments, a tissue section, e.g., a tumor tissue sample, is obtained from a subject by fine needle aspiration, core needle biopsy, stereotactic core needle biopsy, vacuum-assisted core biopsy, or surgical biopsy. In particular embodiments, the surgical biopsy is an incisional biopsy, which removes only part of the suspicious area. In other embodiments, the surgical biopsy is an excisional biopsy, which removes the entire diseased tissue (e.g., tumor) or abnormal area. In particular embodiments, an excisional tumor tissue sample is obtained from a tumor that has been excised with the intent to “cure” a patient in the case of early stage disease, wherein in other embodiments, the excisional tumor tissue sample is obtained from an excised bulk of primary tumor in later stage disease. Tumor tissue samples may include primary tumor tissue, metastatic tumor tissue and/or secondary tumor tissue. Tumor tissue samples may be cell cultures, e.g., cultures of tumor-derived cell lines. In certain embodiments, a tissue section is a cell line, e.g., a cell pellet of a cultured cell line, such as a tumor cell line. In particular embodiments, the cell line or cell pellet is frozen or was previously frozen. Such cell lines and pellets are useful, e.g., as positive or negative controls for imaging with various reagents. Tumor tissue samples may also be xenograft tumors, e.g., tumors obtained from animals administered with tumor cells, e.g., a human tumor cell line. In certain embodiments, a first tumor tissue sample from a subject is a primary tumor tissue sample obtained during an initial surgery intended to remove the entire tumor, and a second tumor tissue sample is obtained from the same subject is a metastatic tumor tissue sample or a secondary tumor tissue sample obtained during a later surgery.

Tissue sections, e.g., tumor tissue samples, may be obtained surgically or using a laparoscope. A tissue section may be a tissue sample obtained from any part of the body to examine it for disease or injury, e.g., presence of cancer tissue or cells, or the extent or characteristics thereof. In particular embodiments, the tissue section includes abdominal tissue, bone, bone marrow, breast tissue, endometrial tissue, kidney tissue, liver tissue, lung or chest tissue, lymph node, nerve tissue, skin, testicular tissue, head or neck tissue, or thyroid tissue. In certain embodiments, the tissue is obtained from brain, breast, skin, bone, joint, skeletal muscle, smooth muscle, red bone marrow, thymus, lymphatic vessel, thoracic duct, spleen, lymph node, nasal cavity, pharynx, larynx, trachea, bronchus, lung, oral cavity, esophagus, liver, stomach, small intestine, large intestine, rectum, anus, spinal cord, nerve, pineal gland, pituitary gland, thyroid gland, thymus, adrenal gland, pancreas, ovary, testis, heart, blood vessel, kidney, uterus, urinary bladder, urethra, prostate gland, penis, prostate, testis, scrotum, ductus deferens, mammary glands, ovary, uterus, vagina, or uterine tube.

In particular embodiments, a tissue section has a size greater than sections typically examined by traditional pathology thin section or immunohistochemical analysis, which are typically in the range of 4-10 microns thick. In certain embodiments, a tissue section is greater than 20 microns, greater than 50 microns, greater than 100 microns, greater than 200 microns, greater than 500 microns, greater than 1 mm, greater than 2 mm, greater than 5 mm, greater than 10 mm or greater than 20 mm in thickness and/or length. In particular embodiments, the tissue section has a length and/or a thickness between 20 microns and 20 mm, between 20 microns and 10 mm, or between 50 microns and 1 mm. In certain embodiments, a tissue section is a cubic sample with each side greater than 10 microns, greater than 20 microns, greater than 50 microns, greater than 100 microns, greater than 200 microns, greater than 500 microns, greater than 1 mm, greater than 2 mm, greater than 5 mm, greater than 10 mm, or greater than 2 mm in thickness and/or length. In some embodiments, a tissue section is thinner, e.g., from about 4-10 or 4-20 microns in thickness.

In embodiments, the tissue section is embedded in an embedding material including paraffin wax, polyepoxide polymer, polyacrylic polymer, agar, gelatin, celloidin, cryogel, optimal cutting temperature (OCT) compositions, glycols, or a combination thereof. In embodiments, the tissue section is embedded in an embedding material including paraffin wax. In embodiments, the OCT composition includes about 10% polyvinyl alcohol and about 4% polyethylene glycol. In embodiments, the OCT composition includes sucrose (e.g., 30% sucrose). In embodiments, the OCT composition is Tissue Freezing Medium (TFM) available from Leica Microsystems, Catalog #14020108926.

In embodiments, the tissue section is an artificial tissue section, wherein the artificial tissue section includes one or more cells suspended in a hydrogel. In embodiments, the artificial tissue section includes one or more cells suspended in a hydrogel that is embedded in an optimal cutting temperature (OCT) composition. In embodiments, the artificial tissue section is prepared according to the following method: the sample containing the biomolecule of interest (e.g., a cell or a particle) is embedded in a crosslinked hydrogel (e.g., a polymer composition including 3 to 20% acrylamide and N,N-dimethylacrylamide). Any suitable hydrogel may be used, for example a hydrogel including poly(2-hydroxyethyl methacrylate) (PHEMA), optionally crosslinked with polyethylene glycol dimethacrylate; 2-hydroxyethyl methacrylate (HEMA) optionally crosslinked with TEGDMA (triethylene glycol dimethacrylate); polyethylene glycol methacrylate (PEGMA), optionally crosslinked with TEGDMA (triethylene glycol dimethacrylate); a copolymer of methacrylic acid (MAA) and polyethylene glycol methacrylate (PEGMA), optionally crosslinked with tetra(ethylene glycol) dimethacrylate; or poly(N-isopropyl acrylamide) (PNIPAM), optionally crosslinked with N,N-methylene bisacrylamide. Additional hydrogels include a polymer such as poly(hydroxyethyl methacrylate) (PHEMA), poly(glyceryl methacrylate) (PGMA), poly(hydroxypropyl methacrylate) (PHPMA), polyacrylamide (PAM), polymethacrylamide (PMAM), polyvinyl alcohol (PVA), polyacrylic acid (PAA), polyvinyl pyrrolidone (PVP), poly(F-caprolactone) (PCL), poly(ethyleneimine) (PEI), poly(N,N-dimethylacrylamide) (PDMAM), poly(2-methoxyethyl acrylate) (PMEA), or a copolymer thereof. Polymer chains in a hydrogel may be crosslinked with each other chemically via covalent bonds or physically via non-covalent interactions to produce the network structure. The physical cross-linking involves hydrogen bonding, hydrophobic interactions, crystallinity, and ionic interactions. In chemically cross-linked hydrogels, covalent bonds cross-link individual polymer chains. Any suitable crosslinker may be used, for example N,N-methylene bisacrylamide, N,N-ethylene bisacrylamide, 1,4-Bis(acryloyl)piperazine, triethylene glycol dimethacrylate (TEGDMA), 1,1,1-trimethylolpropane trimethacrylate (TMPTMA), poly(ethylene glycol) dimethacrylate (PEGDMA), glyoxal, or tetramethylethylenediamine or N,N′-Bis(acryloyl)cystamine.

Following hydrogel embedding, the sample may be frozen in OCT at −80° C. The frozen OCT-hydrogel complex may be then sectioned (e.g., tissue sections of 5 μm and 9 μm thickness). It is known that OCT compounds may impact PCR amplification, see, for example, Turbett and Sellner (Diagn Mol Pathol. 1997 October; 6(5):298-303), so embedding the biological sample in a hydrogel first helps protect the sample from downstream effects from the OCT.

In embodiments, the tissue section is embedded in an embedding material including a polyepoxide polymer. In embodiments, the tissue section is embedded in an embedding material including polyacrylic polymer. In embodiments, the tissue section is embedded in an embedding material including agar. In embodiments, the tissue section is embedded in an embedding material including gelatin. In embodiments, the tissue section is embedded in an embedding material including celloidin. In embodiments, the tissue section is embedded in an embedding material including a cryogel. In embodiments, the tissue section is embedded in an embedding material including optimal cutting temperature (OCT) compositions. In embodiments, the tissue section is embedded in an embedding material including one or more glycols.

In embodiments, the method further includes removing the embedding material. In embodiments, the method further includes removing the embedding material. For example, if the embedding material is paraffin wax, the embedding material is removed by contacting the sample-carrier construct with a hydrocarbon solvent, such as xylene or hexane, followed by two or more washes with decreasing concentrations of an alcohol, such as ethanol.

In embodiments, the tissue section is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissue includes those from skin tissue, muscle tissue, bone tissue, organ tissue and the like. In embodiments, the method includes immobilizing the tissue section in situ to a substrate and permeabilized for delivering probes, enzymes, nucleotides and other components required in the reactions. In embodiments, the tissue section includes many cells from a tissue section in which the original spatial relationships of the cells are retained. In embodiments, the tissue section in situ is within a Formalin-Fixed Paraffin-Embedded (FFPE) sample. In embodiments, the tissue section is subjected to paraffin removal methods, such as methods involving incubation with a hydrocarbon solvent, such as xylene or hexane, followed by two or more washes with decreasing concentrations of an alcohol, such as ethanol. The tissue section may be rehydrated in a buffer, such as PBS, TBS or MOPS. In embodiments, the FFPE sample is incubated with xylene and washed using ethanol to remove the embedding wax, followed by treatment with Proteinase K to permeabilized the tissue. In embodiments, the tissue section is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX®, RCL2/KINFix, UMFIX, Glyo-Fixx®, Histochoice®, or PAXgene®. In embodiments, the tissue section is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

In embodiments, the fixed tissue may be frozen tissue. The frozen biological tissue can be fixed using a fixing agent, which is suitably an organic fixing agent. In some embodiments, the fixing agent can be chilled and can be at a temperature of about 0° C. to about 100° C., suitably about zero to about 50° C., or about 1° C. to about 50° C. The fixing agent can be chilled by placing it over a bed of ice to maintain its temperature as close to 0° C. as possible. The frozen biological tissue can be treated with the fixing agent using any suitable technique, suitably by immersing it in the fixing agent for a period of time. Depending on the type and size of the biological tissue sample, the treatment time can range from about 5 minutes to about 60 minutes, suitably about 10 minutes to about 30 minutes, or about 15 minutes to about 25 minutes, or about 20 minutes. In some embodiments, treatment time may be overnight. During fixing, the snap-frozen tissue will thaw but will suitably remain at a low temperature due to the low temperature environment of the fixing agent.

In some embodiments, the type/identity of a fixation agent, the amount/concentration of a fixation agent, the temperature at which it is used, the duration for which it is used, and the like, may be empirically determined or titrated. These parameters, and others, may need to be varied to obtain optimal results for different tissues, for different organisms, or for different days on which an experiment is performed. Insufficient fixation (e.g., too little fixing agent, too low temperature, too short duration) may not, for example, stabilize/preserve the cells/organelles/analytes of tissues. Excess fixation (e.g., too much fixing agent, too high temperature, too long duration) may result in the single biological samples (e.g., cells/nuclei) obtained from the methods not yielding good results in single biological sample (e.g., single-cell or single nucleus) workflows or assays in which the biological samples (e.g., cells or nuclei) are used. Generally, the quality of data obtained in these workflows/assays may be a good measure of the extent of the fixation process.

In some embodiments, the fixative can be diluted in a buffer, e.g., saline, phosphate buffer (PB), phosphate buffered saline (PBS), citric acid buffer, potassium phosphate buffer, etc., usually at a concentration of about 1-10%, e.g., 1%, 2%, 3%, 4%, 5%, 6%, 7%, 8%, or 10%, for example, 4% paraformaldehyde/0.1 M phosphate buffer; 2% paraformaldehyde/0.2% picric acid/0.1 M phosphate buffer; 4% paraformaldehyde/0.2% periodate/1.2% lysine in 0.1 M phosphate buffer; 4% paraformaldehyde/0.05% glutaraldehyde in phosphate buffer; etc. The type of fixative used and the duration of exposure to the fixative can depend on the sensitivity of the molecules of interest in the tissue section to denaturation by the fixative, and can be readily determined using conventional histochemical or immunohistochemical techniques, for example as described in Buchwalow and Bocker. Immunohistochemistry: Basics and Methods. Springer-Verlag Berlin Heidelberg 2010.

During the fixing, the biological tissue sample can be periodically cut into successively smaller segments while it is submerged in the fixation solution, to facilitate perfusion and fixation of the biological tissue sample by the organic fixing agent. For example, the tissue sample may have an initial length, width and/or diameter of about 0.25 cm to about 1.5 cm or may be initially cut into segments having such suitable dimensions. After a first periodic interval, the tissue sample or segments can be cut into smaller segments, and the smaller segments can remain immersed in the fixing agent. This process can be repeated after a second periodic interval, after a third periodic interval, after a fourth periodic interval, and so on. The periodic intervals can range from about 1 to about 10 minutes, or about 2 to about 8 minutes, or about 4 to about 6 minutes. The sum of the periodic intervals can equal the entire fixing time and can range from about 5 to about 60 minutes, or about 10 to about 30 minutes, or about 15 to about 25 minutes, for example. The resulting fixed tissue segments can have a length, width and/or diameter in a range of less than 1 mm to about 10 mm, by way of example. In some embodiments, the tissue is not cut into smaller segments during fixation. In some embodiments, this may be performed prior to fixation. In some embodiments, this may be performed after fixation.

Once the biological tissue segments have been sufficiently fixed, the fixation process may be stopped and/or the tissue may be removed from the fixation and the tissue may be washed. Generally, fixation is stopped to cease additional activity of the fixative on the tissue. Fixation may also be stopped so that any subsequent biochemical reactions performed on the tissue (e.g., enzymatic cell dissociation) can function. In some embodiments, the tissue segments may be treated or contacted with a halting medium to quench the fixation. The term “halting” means to stop the fixation reaction, i.e., the chemical interactions that cause the fixation. Halting the fixation can be accomplished by immersing the fixed tissue segments in a suitable halting medium. The fixation halting medium can be chilled and can have a temperature of about 0° C. to about 100° C., or about 1° C. to about 50° C. In embodiments, the halting medium is a phosphate buffer solution (PBS). One suitable phosphate buffer solution is 1×PBS, available from Sigma Aldrich Corp. 1×PBS has a pH of about 7.4 and the following composition in water: NaCl—137 mM, KCl—2.7 mM, Na₂HPO₄—10 mM, KH₂PO₄—1.8 mM. In one embodiment, the phosphate buffer solution can be combined with fetal bovine serum (FBS) to aid in halting the fixation reaction. FBS is the liquid fraction of clotted blood from fetal calves, depleted of cells, fibrin and clotting factors, but containing many nutritional and macromolecular factors essential for cell growth. Bovine serum albumin (BSA) is the major component of FBS. The fetal bovine serum can be combined with the phosphate buffer solution at a concentration of about 1% to about 25% by weight FBS and about 75% to about 99% by weight PBS, suitably about 5% to about 15% by weight FBS and about 85% to about 95% by weight PBS, or about 10% by weight FBS and about 90% by weight PBS. In another embodiment, a solution of concentrated ethanol in water can be used instead of the PBS in the halting medium. The ethanol solution can contain about 50% to about 90% by weight ethanol, or about 55% to about 85% by weight ethanol, or about 60% to about 80% by weight ethanol, or about 70% by weight ethanol. In some embodiments, fixation may be quenched using a halting solution that does not contain serum. In some examples, Tris-based buffers may be used. In some examples, PBS+50 mM Tris pH 8.0+0.02% BSA (RNAse free)+0.1 U/μl of RNAse Inhibitor may be used. In some examples, the tissue may be removed from the fixative and washed using a halting solution or biological buffer.

In embodiments, the tissue section is lysed to release nucleic acid or other materials from the cells. For example, the tissue section may be lysed using reagents (e.g., a surfactant such as Triton-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the tissue section.

In embodiments, a permeabilization solution can contain additional reagents or a biological sample may be treated with additional reagents in order to optimize biological sample permeabilization. In some embodiments, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of an RNA protectant includes organic solvents (e.g., at least 60%, 65%, 70%, 75%, 80%, 85%, 90%, or 95% v/v organic solvent), which includes ethanol, methanol, propan-2-ol, acetone, trichloroacetic acid, propanol, polyethylene glycol, acetic acid, or a combination thereof. In embodiments, the RNA protectant includes ethanol, methanol and/or propan-2-ol, or a combination thereof. In embodiments, the RNA protectant includes RNAlater ICE (ThermoFisher Scientific). In embodiments, the RNA protectant includes a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the biological sample is treated with one or more RNA protectants before, contemporaneously with, or after permeabilization.

In embodiments, the analyte, alternatively referred to herein as a biomolecule or target, may be a peptide, protein, or glycoprotein. In embodiments, the analyte is an amino acid, carbohydrate, nucleic acid, lipid, or toxin. In embodiments, the analyte is a steroid. In embodiments, the analyte is a vitamin. In embodiments, the analyte is a virus or virus particles. In embodiments, the analyte is an apoptotic cell. Analytes to be detected also include, but are not limited to, neurotransmitters, hormones, growth factors, antineoplastic agents, cytokines, monokines, lymphokines, nutrients, enzymes, receptors, antibacterial agents, antiviral agents and antifungal agents, and combinations thereof. In embodiments, the analyte is a molecule (e.g., organic or inorganic molecule). In embodiments, the analyte will have at least one epitope that an antibody or a binding agent can recognize. In embodiments, the biomolecule is a lipid, carbohydrate, peptide, protein, or antigen binding fragment. In embodiments, the biomolecule is a lipid. In embodiments, the biomolecule is a carbohydrate. In embodiments, the biomolecule is a peptide. In embodiments, the biomolecule is a protein. In embodiments, the biomolecule is antigen binding fragment. In embodiments, the biomolecule is a subcellular organelle. In embodiments, the subcellular organelle includes a nucleus, nucleoid, mitochondria, endoplasmic reticulum (ER), rough endoplasmic reticulum, smooth endoplasmic reticulum, Golgi apparatus, lysosomes, peroxisomes, ribosomes, cytoskeleton, microfilaments, intermediate filaments, microtubules, plasma membrane, chloroplasts (in plant cells and some protists), vacuoles, centrosomes and centrioles, nucleolus, nuclear envelope, nuclear pores, or transport vesicles.

In embodiments, the method further includes permeabilizing the tissue or cell prior to binding the probe to the biomolecule. Methods for permeabilization are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the tissue and/or cell is cleared (e.g., digested) of proteins, lipids, or proteins and lipids. In embodiments, permeabilizing the tissue or cell does not release the biomolecules (e.g., the one or more biomolecules) from within the tissue or cell. For example, after a fixation process (e.g. formaldehyde cross-linking), proteins and nucleic acids are immobilized within the cells of a tissue section, and are therefore not liberated into the environment following permeabilization of the cells.

Imaging deep into a tissue volume is problematic due to inherently fluorescent molecules present in the tissue or introduced during processing which give rise to autofluorescence that masks fluorescently labelled structures of interest. Typically, autofluorescence decreases image quality by lowering the signal to noise ratio across multiple fluorescence channels and undermines sharp images. Autofluorescence may arise from endogenous fluorescent biomolecules (NADPH, collagen, flavins, tyrosine, and others) or be introduced by the formation of Schiffs bases during fixation with aldehydes (e.g., glutaraldehyde and paraformaldehyde). Additional light scattering is provided by various cellular components, such as ribosomes, nuclei, nucleoli, mitochondria, lipid droplets, membranes, myelin, cytoskeletal components, and extracellular matrix components such as collagen and elastin.

In embodiments, the tissue or cell is cleared using a solvent-based clearing approach. Solvent-based clearing techniques typically includes two steps: 1) dehydration (e.g., contacting the sample with methanol with or without hexane or, tetrahydrofuran (THF) alone) and 2) clearing by refractive index matching to the remaining dehydrated tissue's index (e.g., contacting the tissue sample with methyl salicylate, benzyl alcohol, benzyl benzoate, dichloromethane, or dibenzyl ether). Alternatively, the initial dehydration may be performed using phosphate buffered saline (PBS), detergent, and dimethyl sulfoxide (DMSO). In embodiments, the tissue is cleared by contacting the tissue sample with an aqueous solution containing sucrose, fructose, 2,2′-thiodiethanol (TDE), or formamide.

In embodiments, the tissue or cell is cleared utilizing the 3D imaging of solvent-cleared organs (3DISCO) method as described in Erturk A et al. Nat Protoc. 2012 November; 7(11):1983-95, which is incorporated herein by reference. For example, a sample is incubated overnight in 50% v/v tetrahydrofuran/H₂O (THF), followed by incubation for at least one hour 80% THF/H₂O and followed by incubation in a 100% THF solution. This is then followed by contacting the sample with dichloromethane (DCM) and an incubation in dibenzyl ether (DBE) until clear.

In embodiments, the tissue or cell is cleared according to a known technique in the art, for example CLARITY (Chung K., et al. Nature 497, 332-337 (2013)), PACT-PARS (Yang B et al. Cell 158, 945-958 (2014).), CUBIC (Susaki E. A. et al. Cell 157, 726-739 (2014)., 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, e0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

In embodiments, the tissue or cell is contacted with an alkaline solution containing a combination of 2,2′-thiodiethanol (TDE), DMSO, D-sorbitol, and Tris. In embodiments, the tissue section is contacted with an aqueous solution including 20% (vol/vol) DMSO, 40% (vol/vol) TDE, 20% (wt/vol) sorbitol, and 6% (wt/vol, equal to 0.5 M) Tris base. In embodiments, the tissue section is contacted with an aqueous solution including 25% (wt/wt) urea, 25% (wt/wt) N,N,N′,N′-Tetrakis (2-hydroxypropyl) ethylenediamine, and 15% (wt/wt) Triton X-100. In embodiments, the tissue section is contact with an aqueous solution including 9.1 M urea, 22.5% (wt/vol) D-sorbitol, and 5% (wt/vol) Triton X-100. In embodiments, the tissue section is contact with an aqueous solution including 30% (wt/vol) urea, 20% (wt/vol) D-sorbitol, and 5% (wt/vol) glycerol dissolved in DMSO. In embodiments, the tissue or cell is contacted with an aqueous solution according to the protocols described in Shan, Q H., Qin, X Y., Zhou, N. et al. BMC Biol 20, 77 (2022).

In embodiments, the biological sample (i.e., the tissue or cell) can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). In embodiments, the biological sample can be permeabilized by contacting the sample with a permeabilization solution. In some embodiments, the biological sample is permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, the biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In embodiments, the cell or tissue is immobilized to a solid support. For example, in embodiments, the method includes generating an immobilized biological sample, wherein generating an immobilized biological sample includes forming a plurality of covalent bonds between the biological sample and the receiving substrate (i.e., the solid support). In embodiments, the plurality of covalent bonds includes amide and imide bonds. In embodiments, the plurality of covalent bonds includes amide bonds. In embodiments, the plurality of covalent bonds includes imide bonds. In embodiments, the receiving substrate includes (3-aminopropyl)triethoxysilane (APTES), (3-Aminopropyl)trimethoxysilane (APTMS), γ-Aminopropylsilatrane (APS), N-(6-aminohexyl)aminomethyltriethoxysilane (AHAMTES), polyethylenimine (PEI), 5,6-epoxyhexyltriethoxysilane, or triethoxysilylbutyraldehyde, or a combination thereof. In embodiments, the receiving substrate includes (3-aminopropyl)triethoxysilane (APTES). In embodiments, the receiving substrate includes (3-Aminopropyl)trimethoxysilane (APTMS). In embodiments, the receiving substrate includes y-Aminopropylsilatrane (APS). In embodiments, the receiving substrate includes N-(6-aminohexyl)aminomethyltriethoxysilane (AHAMTES). In embodiments, the receiving substrate surface includes polyethylenimine (PEI). In embodiments, the receiving substrate includes 5,6-epoxyhexyltriethoxysilane. In embodiments, the receiving substrate includes triethoxysilylbutyraldehyde. In embodiments, the receiving substrate is a functionalized glass surface or a functionalized plastic surface. In embodiments, the functionalized glass surface is functionalized with APTES, APTMS, APS, or AHAMTES. In embodiments, the receiving substrate includes a functionalized glass surface or a functionalized plastic surface. Functionalization, as used herein, refers to a modification of the original surface. For example, functionalization may include topographical modifications (e.g., groves, posts, etching), chemical modifications (e.g., binding one or more compounds to the surface to alter the surface charge or bioconjugate reactive moieties on the surface), biological modifications (e.g., immobilizing one or more heparin proteins, heparin sulfate binding proteins, peptide sequences, growth factors, fibronectin, laminin, or collagen), or plasma treatment on reactive glass to generate bioconjugate reactive moieties on the surface.

In embodiments, the receiving substrate is functionalized with an RGD peptide or YIGSR peptide. RGD peptide is one of the most physiologically ubiquitous binding motifs commonly used, which is found in many natural adhesive proteins such as fibronectin, vitronectin, laminin and collagen type I.

In embodiments, the receiving substrate is functionalized with one or more synthetic chemical molecules. In embodiments, the receiving substrate includes dimethyl sulfoxide (DMSO), all-trans retinoic acid (RA), dynorphin B, ascorbic acid. In embodiments, the receiving substrate includes one or more bioconjugate reactive moieties (e.g., carboxyl or amine groups) on the surface of the receiving substrate.

In embodiments, the first probe includes a first detectable label attached to the first probe via a cleavable linker, wherein the cleavable linker of the first probe includes the same cleavable mechanism (e.g., is cleaved upon exposure to the same cleaving agent) as the first cleavable linker tethering the quenching moiety to the second probe. Common quenching moieties in the visible range include monovalent moieties including a guanidine moiety, guanidine thiocyanate moiety, dihydroxyacetone moiety, dinitrophenylhydrazine moiety, and cysteamine moiety. Guanidine is a small molecule that can effectively quench fluorophores in a broad range of wavelengths from short-wavelength blue light (400 nm) to middle-wavelength red light (675 nm). Guanidine thiocyanate is a derivative of guanidine and has similar quenching capabilities but over a slightly shorter wavelength range. Dihydroxyacetone can quench in the visible range. Its efficiency decreases as the wavelength increases from the blue to the green end of the visible spectrum. Dinitrophenylhydrazine is also a quenching moiety, which works in the range of blue light to orange light. Finally, cysteamine is primarily used to quench in the green to red end of the visible spectrum. Other common quenching moieties include compounds such as acridine, nitriles, thiophenes, thiazoles, perylenebisimidazoles and perylene nitrile.

In embodiments, the oligonucleotide is covalently attached to a protein-specific binding agent, wherein the protein-specific binding agent is an antibody, single-chain Fv fragment (scFv), affimer, aptamer, single-domain antibody (sdAb), or antibody fragment-antigen binding (Fab). In embodiments, the oligonucleotide is covalently attached to an antibody or single-domain antibody (sdAb). In embodiments, the oligonucleotide is covalently attached to an antibody. In embodiments, the oligonucleotide is covalently attached to a single-chain Fv fragment (scFv). In embodiments, the oligonucleotide is covalently attached to an affimer. In embodiments, the oligonucleotide is covalently attached to an aptamer. In embodiments, the oligonucleotide is covalently attached to a single-domain antibody (sdAb). In embodiments, the oligonucleotide is covalently attached to an antibody fragment-antigen binding (Fab). In embodiments, the oligonucleotide is covalently attached to an antibody or single-domain antibody (sdAb). The design and preparation of protein-specific binding agent oligonucleotide conjugates is known, for example various different binding moieties which may be used, the design of probe oligonucleotides, and the coupling of such oligonucleotides to the binding moieties to form the conjugates. The details and principles may be applied to the design of the probes for use in the methods described herein. For example, reference may be made to WO 2007/107743, U.S. Pat. Nos. 7,306,904 and 6,878,515 which are incorporated herein by reference.

In embodiments, the oligonucleotide (e.g., the first oligonucleotide and/or the second oligonucleotide) is attached to a specific binding agent (e.g., an antibody) via a linker (e.g., a bioconjugate linker). In embodiments, the oligonucleotide is attached to the protein-specific binding agent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety). For example, oligonucleotides may be covalently attached using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. In embodiments, the oligonucleotide includes a barcode, wherein the barcode is a known sequence associated with the specific binding reagent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, the method includes repeating steps (a)-(c). In embodiments, the method includes repeating steps (a)-(c), wherein one series of steps (a)-(c) is referred to as a cycle, and the method includes 1 to 30 cycles.

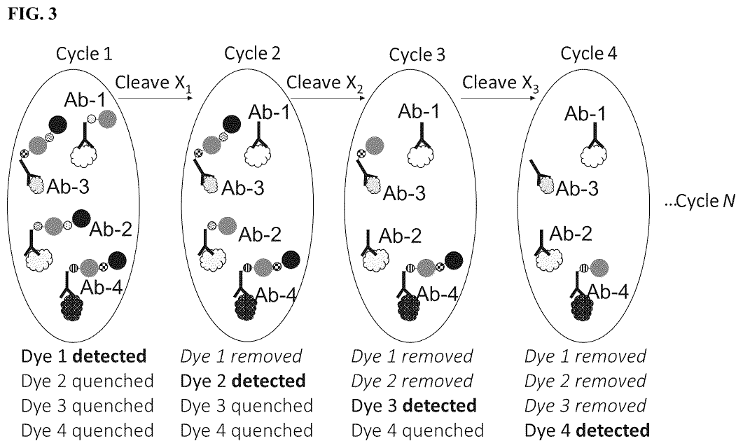

In embodiments, the method further includes contacting the cell or tissue with a third probe and forming a third complex, wherein the third complex includes the third probe bound to a third biomolecule, wherein the third probe includes a third detectable label and a quenching moiety, wherein the third detectable label is attached to the third probe via a third linker; and the quenching moiety is attached to the third probe via a second cleavable linker; and the method further includes cleaving the second cleavable linker; and detecting the third complex. For example, FIG. 3 depicts the serial detection of a third complex in Cycle 3 following the detection of the first complex and cleavage of the first cleavable linker in Cycle 1 and detection of the second complex and cleavage of the second cleavable linker in Cycle 2.

In embodiments, the first detectable label is a fluorescent moiety and the second detectable label is the same fluorescent moiety (i.e., the first and second detectable labels include fluorophores of the same type). In embodiments, the first detectable label is a fluorescent moiety and the second detectable label is a different fluorescent moiety (i.e., the first and second detectable labels include spectrally distinct fluorophores). In embodiments, the first detectable label generates a first signal and the second detectable label generates a second signal, wherein the first signal and second signal are the same. In embodiments, the first detectable label generates a first signal and the second detectable label generates a second signal, wherein the first signal and second signal are spectrally different. Quantification of the label may include a measure of fluorescence intensity, which may be compared to reference values or an internal control. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Atto® dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.).

In embodiments, the detectable label is an iFluor® dye. In embodiments, the detectable label is fluorescent moiety. In embodiments, the detectable label is a fluorescent moiety including an excitation and emission wavelength of 344 nm and 488 nm, such as iFluor® 350 or Coumarin, Alexa Fluor® 350, or DyLight™ 350. In embodiments, the detectable label is a fluorescent moiety including an excitation and emission wavelength of 402 nm and 425 nm, such as iFluor® 405 or Cascade Blue®, Alexa Fluor® 405, or DyLight™ 405. In embodiments, the detectable label is a fluorescent moiety including an excitation and emission wavelength of 433 nm and 495 nm, such as iFluor® 430 or Alexa Fluor® 430. In embodiments, the detectable label is a fluorescent moiety including an excitation and emission wavelength of 434 nm and 480 nm, such as iFluor® 440. In embodiments, the detectable label is a fluorescent moiety including an excitation and emission wavelength of 451 nm and 502 nm, such as iFluor® 450. In embodiments, the detectable label is a fluorescent moiety including an excitation and emission wavelength of 492 nm and 516 nm, such as iFluor® 488 or Alexa Fluor® 488, or DyLight™ 488. In embodiments, the detectable label is a fluorescent moiety including an excitation and emission wavelength of 527 nm and 554 nm, such as iFluor® 514 or Alexa Fluor® 514. In embodiments, the detectable label is a fluorescent moiety including an excitation and emission wavelength of 543 nm and 563 nm, such as iFluor® 532 or Alexa Fluor® 532. In embodiments, the detectable label is a fluorescent moiety including an excitation and emission wavelength of 541 nm and 557 nm, such as iFluor® 546 or Alexa Fluor® 546. In embodiments, the detectable label is a fluorescent moiety including an excitation and emission wavelength of 556 nm and 569 nm, such as iFluor® 555 or Cy3®, Alexa Fluor® 555, or DyLight™ 550. In embodiments, the detectable label is a fluorescent moiety including an excitation and emission wavelength of 559 nm and 571 nm, such as iFluor® 560. In embodiments, the detectable label is a fluorescent moiety including an excitation and emission wavelength of 568 nm and 587 nm, such as iFluor® 568 or Rhodamine Red, Alexa Fluor® 568. In embodiments, the detectable label is a fluorescent moiety including an excitation and emission wavelength of 587 nm and 603 nm, such as iFluor® 594 or Texas Red®, Alexa Fluor® 594, or DyLight™ 594. In embodiments, the detectable label is a fluorescent moiety including an excitation and emission wavelength of 609 nm and 627 nm, such as iFluor® 610 or Alexa Fluor® 610. In embodiments, the detectable label is a fluorescent moiety including an excitation and emission wavelength of 638 nm and 652 nm, such as iFluor® 633 or Alexa Fluor® 633. In embodiments, the detectable label is a fluorescent moiety including an excitation and emission wavelength of 654 nm and 669 nm, such as iFluor® 647 or Cy5®, Alexa Fluor® 647, or DyLight™ 650. In embodiments, the detectable label is a fluorescent moiety including an excitation and emission wavelength of 660 nm and 677 nm, such as iFluor® 660. In embodiments, the detectable label is a fluorescent moiety including an excitation and emission wavelength of 669 nm and 682 nm, such as iFluor® 670 or Cy5® B. In embodiments, the detectable label is a fluorescent moiety including an excitation and emission wavelength of 683 nm and 700 nm, such as iFluor® 680 or Cy5.5®, IRDye® 700, or Alexa Fluor® 680. In embodiments, the detectable label is a fluorescent moiety including an excitation and emission wavelength of 690 nm and 713 nm, such as iFluor® 700 or Alexa Fluor® 700. In embodiments, the detectable label is a fluorescent moiety including an excitation and emission wavelength of 712 nm and 736 nm, such as iFluor® 710. In embodiments, the detectable label is a fluorescent moiety including an excitation and emission wavelength of 759 nm and 777 nm, such as iFluor® 750 or Cy7®, Alexa Fluor® 750, or DyLight™ 750. In embodiments, the detectable label is a fluorescent moiety including an excitation and emission wavelength of 786 nm and 811 nm, such as iFluor® 790 or IRDye® 800, Alexa Fluor® 790, or DyLight™ 800. In embodiments, the detectable label is a fluorescent moiety including an excitation and emission wavelength of 801 nm and 820 nm, such as iFluor® 800. In embodiments, the detectable label is a fluorescent moiety including an excitation and emission wavelength of 812 nm, such as iFluor® 810. In embodiments, the detectable label is a fluorescent moiety including an excitation and emission wavelength of 820 nm, such as iFluor® 820.

In embodiments, detecting includes imaging. In embodiments, detecting includes obtaining an image. In embodiments, detecting includes detecting the detectable label attached to the probe described herein (e.g., Dye1, Dye2, and/or Dye3) at the maximum emission wavelength of the detectable label. In embodiments, the maximum emission wavelength of the detectable label (also referred herein as “fluorescent moiety”) is about 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, 430 nm, 431 nm, 432 nm, 433 nm, 434 nm, 435 nm, 436 nm, 437 nm, 438 nm, 439 nm, 440 nm, 441 nm, 442 nm, 443 nm, 444 nm, 445 nm, 446 nm, 447 nm, 448 nm, 449 nm, 450 nm, 451 nm, 452 nm, 453 nm, 454 nm, 455 nm, 456 nm, 457 nm, 458 nm, 459 nm, 460 nm, 461 nm, 462 nm, 463 nm, 464 nm, 465 nm, 466 nm, 467 nm, 468 nm, 469 nm, 470 nm, 471 nm, 472 nm, 473 nm, 474 nm, 475 nm, 476 nm, 477 nm, 478 nm, 479 nm, 480 nm, 481 nm, 482 nm, 483 nm, 484 nm, 485 nm, 486 nm, 487 nm, 488 nm, 489 nm, 490 nm, 491 nm, 492 nm, 493 nm, 494 nm, 495 nm, 496 nm, 497 nm, 498 nm, 499 nm, 500 nm, 501 nm, 502 nm, 503 nm, 504 nm, 505 nm, 506 nm, 507 nm, 508 nm, 509 nm, 510 nm, 511 nm, 512 nm, 513 nm, 514 nm, 515 nm, 516 nm, 517 nm, 518 nm, 519 nm, 520 nm, 521 nm, 522 nm, 523 nm, 524 nm, 525 nm, 526 nm, 527 nm, 528 nm, 529 nm, 530 nm, 531 nm, 532 nm, 533 nm, 534 nm, 535 nm, 536 nm, 537 nm, 538 nm, 539 nm, 540 nm, 541 nm, 542 nm, 543 nm, 544 nm, 545 nm, 546 nm, 547 nm, 548 nm, 549 nm, 550 nm, 551 nm, 552 nm, 553 nm, 554 nm, 555 nm, 556 nm, 557 nm, 558 nm, 559 nm, 560 nm, 561 nm, 562 nm, 563 nm, 564 nm, 565 nm, 566 nm, 567 nm, 568 nm, 569 nm, 570 nm, 571 nm, 572 nm, 573 nm, 574 nm, 575 nm, 576 nm, 577 nm, 578 nm, 579 nm, 580 nm, 581 nm, 582 nm, 583 nm, 584 nm, 585 nm, 586 nm, 587 nm, 588 nm, 589 nm, 590 nm, 591 nm, 592 nm, 593 nm, 594 nm, 595 nm, 596 nm, 597 nm, 598 nm, 599 nm, 600 nm, 601 nm, 602 nm, 603 nm, 604 nm, 605 nm, 606 nm, 607 nm, 608 nm, 609 nm, 610 nm, 611 nm, 612 nm, 613 nm, 614 nm, 615 nm, 616 nm, 617 nm, 618 nm, 619 nm, 620 nm, 621 nm, 622 nm, 623 nm, 624 nm, 625 nm, 626 nm, 627 nm, 628 nm, 629 nm, 630 nm, 631 nm, 632 nm, 633 nm, 634 nm, 635 nm, 636 nm, 637 nm, 638 nm, 639 nm, 640 nm, 641 nm, 642 nm, 643 nm, 644 nm, 645 nm, 646 nm, 647 nm, 648 nm, 649 nm, 650 nm, 651 nm, 652 nm, 653 nm, 654 nm, 655 nm, 656 nm, 657 nm, 658 nm, 659 nm, 660 nm, 661 nm, 662 nm, 663 nm, 664 nm, 665 nm, 666 nm, 667 nm, 668 nm, 669 nm, 670 nm, 671 nm, 672 nm, 673 nm, 674 nm, 675 nm, 676 nm, 677 nm, 678 nm, 679 nm, 680 nm, 681 nm, 682 nm, 683 nm, 684 nm, 685 nm, 686 nm, 687 nm, 688 nm, 689 nm, 690 nm, 691 nm, 692 nm, 693 nm, 694 nm, 695 nm, 696 nm, 697 nm, 698 nm, 699 nm, 700 nm, 701 nm, 702 nm, 703 nm, 704 nm, 705 nm, 706 nm, 707 nm, 708 nm, 709 nm, 710 nm, 711 nm, 712 nm, 713 nm, 714 nm, 715 nm, 716 nm, 717 nm, 718 nm, 719 nm, 720 nm, 721 nm, 722 nm, 723 nm, 724 nm, 725 nm, 726 nm, 727 nm, 728 nm, 729 nm, 730 nm, 731 nm, 732 nm, 733 nm, 734 nm, 735 nm, 736 nm, 737 nm, 738 nm, 739 nm, 740 nm, 741 nm, 742 nm, 743 nm, 744 nm, 745 nm, 746 nm, 747 nm, 748 nm, 749 nm, 750 nm, 751 nm, 752 nm, 753 nm, 754 nm, 755 nm, 756 nm, 757 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 788 nm, 789 nm, 790 nm, 791 nm, 792 nm, 793 nm, 794 nm, 795 nm, 796 nm, 797 nm, 798 nm, 799 nm, or 800 nm. In embodiments, the maximum emission wavelength of the detectable label (also referred herein as “fluorescent moiety”) is about 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 nm, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1000 nm, 1001 nm, 1002 nm, 1003 nm, 1004 nm, 1005 nm, 1006 nm, 1007 nm, 1008 nm, 1009 nm, 1010 nm, 1011 nm, 1012 nm, 1013 nm, 1014 nm, 1015 nm, 1016 nm, 1017 nm, 1018 nm, 1019 nm, 1020 nm, 1021 nm, 1022 nm, 1023 nm, 1024 nm, 1025 nm, 1026 nm, 1027 nm, 1028 nm, 1029 nm, 1030 nm, 1031 nm, 1032 nm, 1033 nm, 1034 nm, 1035 nm, 1036 nm, 1037 nm, 1038 nm, 1039 nm, 1040 nm, 1041 nm, 1042 nm, 1043 nm, 1044 nm, 1045 nm, 1046 nm, 1047 nm, 1048 nm, 1049 nm, 1050 nm, 1051 nm, 1052 nm, 1053 nm, 1054 nm, 1055 nm, 1056 nm, 1057 nm, 1058 nm, 1059 nm, 1060 nm, 1061 nm, 1062 nm, 1063 nm, 1064 nm, 1065 nm, 1066 nm, 1067 nm, 1068 nm, 1069 nm, 1070 nm, 1071 nm, 1072 nm, 1073 nm, 1074 nm, 1075 nm, 1076 nm, 1077 nm, 1078 nm, 1079 nm, 1080 nm, 1081 nm, 1082 nm, 1083 nm, 1084 nm, 1085 nm, 1086 nm, 1087 nm, 1088 nm, 1089 nm, 1090 nm, 1091 nm, 1092 nm, 1093 nm, 1094 nm, 1095 nm, 1096 nm, 1097 nm, 1098 nm, 1099 nm, 1100 nm, 1101 nm, 1102 nm, 1103 nm, 1104 nm, 1105 nm, 1106 nm, 1107 nm, 1108 nm, 1109 nm, 1110 nm, 1111 nm, 1112 nm, 1113 nm, 1114 nm, 1115 nm, 1116 nm, 1117 nm, 1118 nm, 1119 nm, 1120 nm, 1121 nm, 1122 nm, 1123 nm, 1124 nm, 1125 nm, 1126 nm, 1127 nm, 1128 nm, 1129 nm, 1130 nm, 1131 nm, 1132 nm, 1133 nm, 1134 nm, 1135 nm, 1136 nm, 1137 nm, 1138 nm, 1139 nm, 1140 nm, 1141 nm, 1142 nm, 1143 nm, 1144 nm, 1145 nm, 1146 nm, 1147 nm, 1148 nm, 1149 nm, 1150 nm, 1151 nm, 1152 nm, 1153 nm, 1154 nm, 1155 nm, 1156 nm, 1157 nm, 1158 nm, 1159 nm, 1160 nm, 1161 nm, 1162 nm, 1163 nm, 1164 nm, 1165 nm, 1166 nm, 1167 nm, 1168 nm, 1169 nm, 1170 nm, 1171 nm, 1172 nm, 1173 nm, 1174 nm, 1175 nm, 1176 nm, 1177 nm, 1178 nm, 1179 nm, 1180 nm, 1181 nm, 1182 nm, 1183 nm, 1184 nm, 1185 nm, 1186 nm, 1187 nm, 1188 nm, 1189 nm, 1190 nm, 1191 nm, 1192 nm, 1193 nm, 1194 nm, 1195 nm, 1196 nm, 1197 nm, 1198 nm, 1199 nm, 1200 nm, 1201 nm, 1202 nm, 1203 nm, 1204 nm, 1205 nm, 1206 nm, 1207 nm, 1208 nm, 1209 nm, 1210 nm, 1211 nm, 1212 nm, 1213 nm, 1214 nm, 1215 nm, 1216 nm, 1217 nm, 1218 nm, 1219 nm, 1220 nm, 1221 nm, 1222 nm, 1223 nm, 1224 nm, 1225 nm, 1226 nm, 1227 nm, 1228 nm, 1229 nm, 1230 nm, 1231 nm, 1232 nm, 1233 nm, 1234 nm, 1235 nm, 1236 nm, 1237 nm, 1238 nm, 1239 nm, 1240 nm, 1241 nm, 1242 nm, 1243 nm, 1244 nm, 1245 nm, 1246 nm, 1247 nm, 1248 nm, 1249 nm, 1250 nm, 1251 nm, 1252 nm, 1253 nm, 1254 nm, 1255 nm, 1256 nm, 1257 nm, 1258 nm, 1259 nm, 1260 nm, 1261 nm, 1262 nm, 1263 nm, 1264 nm, 1265 nm, 1266 nm, 1267 nm, 1268 nm, 1269 nm, 1270 nm, 1271 nm, 1272 nm, 1273 nm, 1274 nm, 1275 nm, 1276 nm, 1277 nm, 1278 nm, 1279 nm, 1280 nm, 1281 nm, 1282 nm, 1283 nm, 1284 nm, 1285 nm, 1286 nm, 1287 nm, 1288 nm, 1289 nm, 1290 nm, 1291 nm, 1292 nm, 1293 nm, 1294 nm, 1295 nm, 1296 nm, 1297 nm, 1298 nm, 1299 nm, 1300 nm, 1301 nm, 1302 nm, 1303 nm, 1304 nm, 1305 nm, 1306 nm, 1307 nm, 1308 nm, 1309 nm, 1310 nm, 1311 nm, 1312 nm, 1313 nm, 1314 nm, 1315 nm, 1316 nm, 1317 nm, 1318 nm, 1319 nm, 1320 nm, 1321 nm, 1322 nm, 1323 nm, 1324 nm, 1325 nm, 1326 nm, 1327 nm, 1328 nm, 1329 nm, 1330 nm, 1331 nm, 1332 nm, 1333 nm, 1334 nm, 1335 nm, 1336 nm, 1337 nm, 1338 nm, 1339 nm, 1340 nm, 1341 nm, 1342 nm, 1343 nm, 1344 nm, 1345 nm, 1346 nm, 1347 nm, 1348 nm, 1349 nm, 1350 nm, 1351 nm, 1352 nm, 1353 nm, 1354 nm, 1355 nm, 1356 nm, 1357 nm, 1358 nm, 1359 nm, 1360 nm, 1361 nm, 1362 nm, 1363 nm, 1364 nm, 1365 nm, 1366 nm, 1367 nm, 1368 nm, 1369 nm, 1370 nm, 1371 nm, 1372 nm, 1373 nm, 1374 nm, 1375 nm, 1376 nm, 1377 nm, 1378 nm, 1379 nm, 1380 nm, 1381 nm, 1382 nm, 1383 nm, 1384 nm, 1385 nm, 1386 nm, 1387 nm, 1388 nm, 1389 nm, 1390 nm, 1391 nm, 1392 nm, 1393 nm, 1394 nm, 1395 nm, 1396 nm, 1397 nm, 1398 nm, 1399 nm, 1400 nm, 1401 nm, 1402 nm, 1403 nm, 1404 nm, 1405 nm, 1406 nm, 1407 nm, 1408 nm, 1409 nm, 1410 nm, 1411 nm, 1412 nm, 1413 nm, 1414 nm, 1415 nm, 1416 nm, 1417 nm, 1418 nm, 1419 nm, 1420 nm, 1421 nm, 1422 nm, 1423 nm, 1424 nm, 1425 nm, 1426 nm, 1427 nm, 1428 nm, 1429 nm, 1430 nm, 1431 nm, 1432 nm, 1433 nm, 1434 nm, 1435 nm, 1436 nm, 1437 nm, 1438 nm, 1439 nm, 1440 nm, 1441 nm, 1442 nm, 1443 nm, 1444 nm, 1445 nm, 1446 nm, 1447 nm, 1448 nm, 1449 nm, or 1450 nm. In embodiments, detecting includes detecting a light emission in the near-infrared spectrum. In embodiments, detecting includes detecting a light emission with a wavelength from 600 nm-900 nm. In embodiments, detecting includes detecting a light emission with a wavelength from 600 nm-1450 nm. In embodiments, detecting includes detecting a light emission with a wavelength from 1000 nm-1700 nm. In embodiments, detecting includes detecting a light emission in the “imaging window,” which refers to a range of wavelengths where tissue autofluorescence is minimal and the absorption and emission of light in tissue results in minimal light scattering (see, e.g., Pansare et al. Chem Mater. 2012 Mar. 13; 24(5): 812-827 and Wang et al. ACS Cent Sci. 2020 Aug. 26; 6(8): 1302-1316).

In embodiments, detecting includes directing an excitation light to the sample (e.g., cell or tissue) and detecting an emission light from the first fluorescent dye, the second fluorescent dye, and/or the stain.

In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moiré pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126, which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

In embodiments, prior to imaging, the method further includes permeabilizing the immobilized biological sample. In alternative embodiments, prior to imaging, the method does not include permeabilizing the immobilized biological section. In embodiments, prior to imaging, the method further includes contacting the immobilized biological sample with one or more imaging reagents or stains. In embodiments, following permeabilization, the biological sample is contacted with one or more imaging reagents or stains. In embodiments, the biological sample is contacted with one or more imaging reagents or stains without permeabilization. In embodiments, the imaging reagents or stains include hematoxylin and eosin (H&E) staining reagents. In embodiments, the imaging (e.g., step E)) includes phase-contrast microscopy, bright-field microscopy, Nomarski differential-interference-contrast microscopy, dark field microscopy, electron microscopy, or cryo-electron microscopy. In embodiments, the imaging reagents or stains include phase-contrast microscopy, bright-field microscopy, Nomarski differential-interference-contrast microscopy, or dark field microscopy imaging reagents. In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor.

In embodiments, the imaging reagents or stains include electron microscopy (e.g., transmission electron microscopy or scanning electron microscopy) or cryo-electron microscopy imaging reagents. Examples of electron microscopy contrast agents may include one or more heavy metals (e.g., gold particles, colloidal gold particles, uranium, lead, platinum, and/or osmium) and/or antibodies bound to one or more types of heavy metals (e.g., gold particles, colloidal gold particles, uranium, lead, platinum, and/or osmium). For example, immunogold labels that may be used to contact the tissue section include may include different antibodies bound to gold particles of different sizes to image different molecules of interest. Optionally, the method may include contacting the tissue section with heavy metals. Heavy metals that may be used to stain additional features of interest and/or provide contrast between different structures in the tissue section may include uranium, lead, platinum, and/or osmium (see, e.g., U.S. Pat. Pubs. 2019/0355550 and 2013/0344500, each of which is incorporated herein by reference in its entirety).

In embodiments, cleaving the cleavable linker includes contacting the cleavable linker with a cleaving agent. In embodiments, the cleaving agent cleaves the cleavable site of the cleavable linker. In embodiments, the cleaving agent cleaves the cleavable site of the covalent linker. In embodiments, the cleaving agent cleaves the cleavable site of the linker (e.g., the first linker, the second linker, the third linker, etc. described herein).

In embodiments, the first cleavable linker and the second cleavable linker are cleaved by the same cleaving agent. In embodiments, the first cleavable linker and the second cleavable linker are cleaved by different cleaving agents and/or different reaction conditions. In embodiments, the first cleavable linker and the second cleavable linker are orthogonal cleavable linkers. For example, the first cleavable linker is shown in FIG. 2A as X₁, and the second cleavable linker is shown in FIG. 2A as X₂.

In embodiments, cleaving the first cleavable linker includes contacting the first cleavable linker with a cleaving agent. In embodiments, the cleaving agent cleaves the cleavable site of the first linker (i.e., the linker tethering the quenching moiety to the second probe). In embodiments, the cleaving agent is a reducing agent or an oxidizing agent.

In embodiments, the linker (e.g., the first linker, the second linker, the third linker, etc. described herein) is a chemically cleavable linker, enzymatically cleavable linker, photo-cleavable linker. In embodiments, the cleavable linker (e.g., the first cleavable linker, the second cleavable linker, the third cleavable linker, etc. described herein) is a chemically cleavable linker, enzymatically cleavable linker, photo-cleavable linker.

In embodiments, the cleavable linker is a hydrazone linker, disulfide linker, peptide linker (such as valine-citrulline), azido linker, β-glucuronide linker, para-aminobenzyl alcohol (PABA) linker, phosphoramidate linker, maleimide ether linker, or triglycine (Gly-Gly-Gly) linker. A hydrazone linker includes

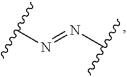 [SMILES: */N=N/*]

which is cleavable with an acidic cleaving agent. In embodiments, the linker includes β-glucuronide, which is cleaved with a β-glucuronidase enzyme. In embodiments, the cleavable linker includes a valine-citrulline (Val-Cit) linker, wherein the Val-Cit linker is cleaved with Cathepsin B. In embodiments, the cleavable linker includes para-aminobenzyl alcohol (PABA) linker, which is cleavable with a para-nitrobenzyl alcohol hydrolase enzyme. In embodiments, the triglycine linker is cleaved with trypsin or chymotrypsin.

In embodiments, the cleavable linker includes a disulfide moiety or an azido moiety. In embodiments, the cleavable linker includes

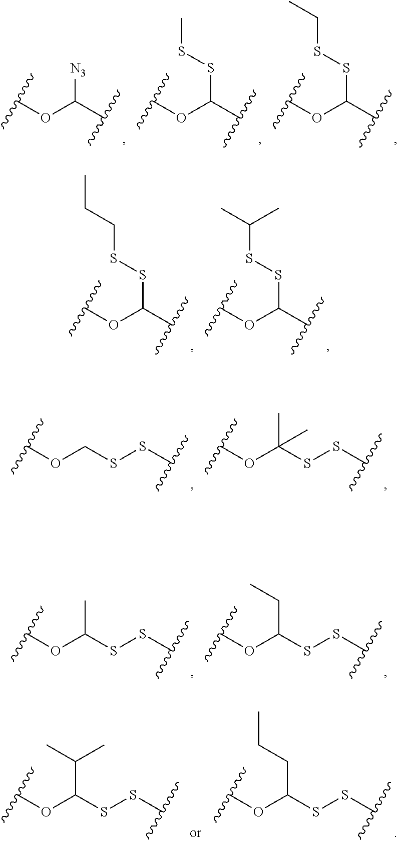

In embodiments, the cleavable linker includes

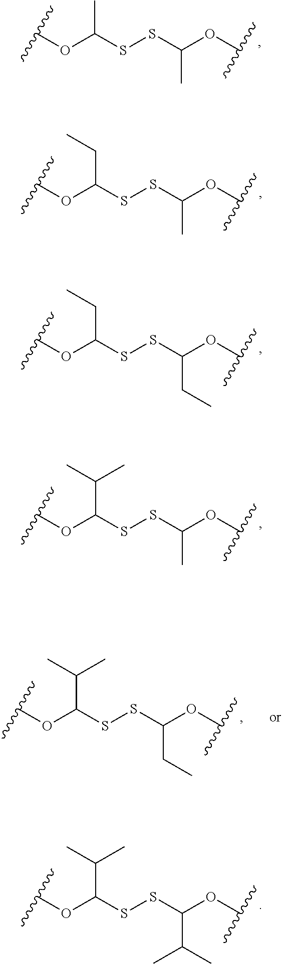

In embodiments, the cleavable linker includes

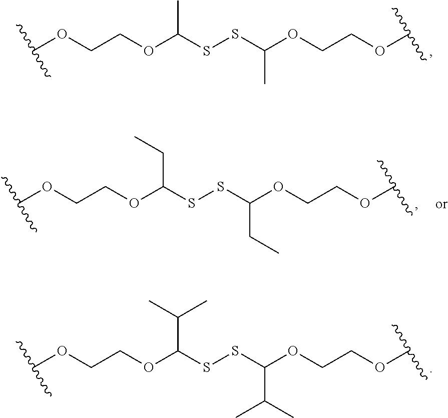 [SMILES: *OCCOC(C)SSC(C)OCCO* ; *OCCOC(CC)SSC(CC)OCCO* ; *OCCOC(SSC(OCCO*)C(C)C)C(C)C]

In embodiments, the cleavable linker is a linker described in U.S. Pat. No. 11,946,103. In embodiments, the cleavable linker is cleaved by contacting the cleavable linker with a reducing agent. In embodiments, the reducing agent is tris(hydroxypropyl) phosphine (THPP), tris-(2-carboxyethyl) phosphine (TCEP), or dithiothreitol (DTT).

In embodiments, the cleavable linker is divalent linker capable of being separated into distinct entities, where one entity remains attached to the specific binding agent (e.g., the probe). A cleavable linker is specifically cleavable in response to external stimuli. In embodiments, the cleavable linker is cleaved by contacting the cleavable linker with a cleaving agent. In embodiments, the cleavable linker includes two or more cleavable sites. Any suitable enzymatic, chemical, or photochemical cleavage reaction may be used to cleave the cleavable site.

In embodiments, the first linker and the first cleavable linker are each a chemically cleavable linker, enzymatically cleavable linker, photo-cleavable linker. In embodiments, the first linker is a chemically cleavable linker, enzymatically cleavable linker, or photo-cleavable linker. In embodiments, the first linker is a chemically cleavable linker. In embodiments, the first linker is an enzymatically cleavable linker. In embodiments, the first linker is a photo-cleavable linker. In embodiments, the first cleavable linker (i.e., the linker tethering the quenching moiety to the second probe) is a chemically cleavable linker, enzymatically cleavable linker, or photo-cleavable linker. In embodiments, the first cleavable linker (i.e., the linker tethering the quenching moiety to the second probe) is a chemically cleavable linker. In embodiments, the first cleavable linker (i.e., the linker tethering the quenching moiety to the second probe) is an enzymatically cleavable linker. In embodiments, the first cleavable linker (i.e., the linker tethering the quenching moiety to the second probe) is a photo-cleavable linker.

In embodiments, the first linker and the first cleavable linker are each a polynucleotide sequence including a restriction site. In embodiments, the first linker is a polynucleotide sequence including a restriction site. In embodiments, the first linker includes a restriction site. In embodiments, the first cleavable linker is a polynucleotide sequence including a restriction site. In embodiments, the first cleavable linker (i.e., the linker tethering the quenching moiety to the second probe) includes a restriction site. In embodiments, the first linker and the first cleavable linker are each an enzymatically cleavable linker. In embodiments, the linker (e.g., the first linker, the second linker, the third linker, etc. described herein) is a polynucleotide sequence including a restriction site. In embodiments, the cleavable linker (e.g., the first cleavable linker, the second cleavable linker, the third cleavable linker, etc. described herein) is a polynucleotide sequence including a restriction site.

In embodiments, the cleavable linker is cleaved by contacting the cleavable linker with a cleaving agent. In embodiments, the cleavable linker can be cleaved by enzymes, nucleophilic/basic reagents, reducing agents, photo-irradiation, electrophilic/acidic reagents, organometallic and metal reagents, or oxidizing reagents. In embodiments, the cleavable linker can be chemically cleaved by a chemical. In embodiments, the chemically cleavable linker is split in response to the presence of a acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, a chemically cleavable linker is non-enzymatically cleavable. In embodiments, cleaving includes removing. In embodiments, the cleavable linker includes one or more cleavable site(s). Any suitable enzymatic, chemical, or photochemical cleavage reaction may be used to cleave the cleavable site. In embodiments, cleaving the cleavable linker can be chemical cleavage of a bond between a deoxyribonucleotide and a ribonucleotide, in which case, the cleavable site may include one or more ribonucleotides. In embodiments, cleaving of the cleavable site can be a chemical reduction of a disulfide linkage with a reducing agent (e.g., THPP or TCEP), in which case, the cleavable site should include an appropriate disulfide linkage; chemical cleavage of a diol linkage with periodate, in which case, the cleavable site should include a diol linkage; generation of an abasic site and subsequent hydrolysis, etc. In embodiments, the linker includes a diol linkage, which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, the cleavable linker includes two or more cleavable sites. Any suitable enzymatic, chemical, or photochemical cleavage reaction may be used to cleave the cleavable site. In embodiments, the cleavable site includes one or more deoxyuracil nucleobases (dUs). In embodiments, the cleavable site includes multiple deoxyuracil nucleobases (dUs). In embodiments, the cleavable site includes a plurality of consecutive nucleobases (dUs). In embodiments, the cleavable site is cleaved as a result of enzymatic cleaving. In embodiments, the cleaving agent is an enzyme. In embodiments, the enzyme is one or more restriction enzymes. The restriction enzyme will recognize a particular restriction site sequences in one or both strands of the cleavable site, resulting in cleavage of the cleavable site. The resulting restriction enzyme digestion may cleave one or both strands of a duplex template. The enzymatic cleavage reaction may result in removal of a part or the whole of the strand being cleaved. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, the first linker and/or the first cleavable linker include one or more cleavable sites. In embodiments, the first linker includes one or more cleavable sites. In embodiments, the linker (e.g., the first linker, the second linker, the third linker, etc. described herein) includes one or more cleavable sites. In embodiments, the cleavable linker (e.g., the first cleavable linker, the second cleavable linker, the third cleavable linker, etc. described herein) includes one or more cleavable sites. In embodiments, the first cleavable linker include one or more cleavable sites. In embodiments, the cleavable site includes one or more deoxyuracil triphosphates (dUTPs), deoxy-8-oxo-guanine triphosphates (d-8-oxoGs), methylated nucleotides, or ribonucleotides. In embodiments, the cleavable site includes one or more deoxyuracil triphosphates (dUTPs). In embodiments, the cleavable site includes one or more deoxy-8-oxo-guanine triphosphates (d-8-oxoGs). In embodiments, the cleavable site includes one or more methylated nucleotides. In embodiments, the cleavable site includes one or more ribonucleotides. The one or more cleavable sites may include a modified nucleotide, ribonucleotide, or a sequence containing a modified or unmodified nucleotide that is specifically recognized by a cleavage agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes, and include modified ribonucleotides and deoxyribonucleotides including abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. In embodiments, the cleavable site includes an abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C.

In embodiments, the first linker and/or the first cleavable linker includes one or more cleavable site nucleotides. In embodiments, the first linker includes one or more cleavable site nucleotides. In embodiments, the first cleavable linker includes one or more cleavable site nucleotides. In embodiments, the linker (e.g., the first linker, the second linker, the third linker, etc. described herein) includes one or more cleavable site nucleotides. In embodiments, the cleavable linker (e.g., the first cleavable linker, the second cleavable linker, the third cleavable linker, etc. described herein) includes one or more cleavable site nucleotides. In embodiments, the first linker and/or the first cleavable linker includes a plurality of cleavable site nucleotides. In embodiments, the first linker includes a plurality of cleavable site nucleotides. In embodiments, the first cleavable linker includes a plurality of cleavable site nucleotides. In embodiments, the linker (e.g., the first linker, the second linker, the third linker, etc. described herein) includes a plurality of cleavable site nucleotides. In embodiments, the cleavable linker (e.g., the first cleavable linker, the second cleavable linker, the third cleavable linker, etc. described herein) includes a plurality of cleavable site nucleotides. The term “cleavable site nucleotide” refers to a nucleotide that allows for controlled cleavage of the polynucleotide strand following contact with a cleaving agent (e.g., uracil DNA glycosylase (UDG)). Additional examples of cleavable site nucleotides include deoxyuracil triphosphates (dUTPs), deoxy-8-oxo-guanine triphosphates (d-8-oxoGs), methylated nucleotides, or ribonucleotides. In embodiments, the cleavable site nucleotide is dUTP and the cleaving agent is UDG. In embodiments, the cleavable site nucleotide is a ribonucleotide and the cleaving agent is RNase. In embodiments, the cleavable site nucleotide is 8-oxo-7,8-dihydroguanine (8oxoG) and the cleaving agent is formamidopyrimidine DNA glycosylase (Fpg). In embodiments, the cleavable site nucleotide is 5-methylcytosine and the cleaving agent is McrBC. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to herein as “cleaving agents.”

In embodiments, the cleaving agent includes a reducing agent, sodium periodate, RNase, Formamidopyrimidine DNA Glycosylase (Fpg), endonuclease, or uracil DNA glycosylase (UDG).

In some embodiments, the cleaving agent includes one or more restriction endonucleases. When employing restriction endonucleases for cleavage, careful selection of the restriction endonuclease is beneficial, given the need for high efficiency cleavage and the fact that efficiency of cleavage can vary significantly according to the specific restriction endonuclease. Using a novel single molecule counting approach, Zhang et al (see, Zhang Y et al. PLoS ONE. 2020. 15(12): e0244464, which is incorporated herein by reference in its entirety) precisely determined the cleavage efficiency of a variety of common restriction enzymes and the CRISPR-Cas9 nuclease. Zhang reported single enzyme digestion efficiencies ranging from as low as 67.12% for NdeI to as high as 99.53% for EcoRI-HF. Importantly, Zhang notes that the duration of digestion has minimal effect on the overall digestion efficiency such that the fraction of digested templates is nearly unchanged after the first 5 minutes of incubation, suggesting that a 5-minute incubation time serves as a reasonable starting point for optimization of many candidate restriction endonucleases.

In embodiments, the cleaving agent includes a single restriction endonuclease. In embodiments, the restriction endonuclease may include XbaI, EcoRI-HF, NheI, BamHI, XcmI, PflMI, BstEII, NcoI, HpaI, BsgI, AfeI, StuI, BsrGI, or a CRISPR-Cas9 nuclease (e.g., to achieve an approximate 95% cleavage or digestion rate, or the cleaving activity). In embodiments, the restriction endonuclease may include XbaI, EcoRI, BamHI, XcmI or BstEII (e.g., to achieve an approximate 98% or greater cleavage or digestion rate, or the cleaving activity). In embodiments, the restriction endonuclease may include EcoRI or XbaI (e.g., to achieve an approximate 99% or greater cleavage or digestion rate, or the cleaving activity). In some embodiments, the efficiency of cleavage may be further improved by inclusion of more than one restriction enzyme recognition site between the adapter (e.g., adapter including a platform primer binding sequence and/or sequencing primer binding sequence) and insert sequence. In some embodiments, multiple restriction endonucleases may be used in combination to precisely tune the cleavage efficiency. For example, in embodiments where >99.5% cleavage efficiency is required, a suitable dual restriction endonuclease cleavage solution may include XbaI (99.25% efficiency, as reported in Zhang) and NdeI (67.12% efficiency, as reported in Zhang), while the library constructs contain recognition sites for both XbaI and NdeI. Here, the estimated combined cleavage efficiency of the dual restriction endonuclease system is approximately 1−(1−0.9925)(1−0.6712)=99.83%.

In embodiments, cleaving includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage is performed with about 1 unit (U) to about 50 U of restriction endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of restriction endonuclease. In embodiments, cleavage is performed with about 5 U to about 10 U of restriction endonuclease. In embodiments, cleavage is performed with about 10 U to about 15 U of restriction endonuclease. In embodiments, cleavage is performed with about 15 U to about 20 U of restriction endonuclease. In embodiments, cleavage is performed with about 20 U to about 25 U of restriction endonuclease. In embodiments, cleavage is performed with about 25 U to about 35 U of restriction endonuclease. In embodiments, cleavage is performed with about 35 U to about 50 U of restriction endonuclease. In embodiments, cleavage is performed with about 1, 2, 3, 4, 5, 6, 7, 8, 9, 10, 11, 12, 13, 14, 15, 16, 17, 18, 19, 20, 21, 22, 23, 24, 25, 30, 35, 40, 45 or 50 U of restriction endonuclease. In embodiments, cleavage is performed with less than about 1 U of restriction endonuclease. In embodiments, cleavage is performed with greater than about 50 U of restriction endonuclease.

In embodiments, the first probe is a specific binding reagent capable of binding to a first biomolecule and the second probe is a specific binding reagent capable of binding to a second biomolecule, wherein the first biomolecule and the second biomolecule are different. In embodiments, the specific binding reagent is an antibody, single-chain Fv fragment (scFv), antibody fragment-antigen binding (Fab), affimer, or an aptamer. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety.

In embodiments, the first probe is an antibody, single-chain Fv fragment (scFv), antibody fragment-antigen binding (Fab), affimer, or an aptamer. In embodiments, the first probe is an antibody. In embodiments, the first probe is a single-chain Fv fragment (scFv). In embodiments, the first probe is an antibody fragment-antigen binding (Fab). In embodiments, the first probe is an affirmer. In embodiments, the first probe is an aptamer. In embodiments, the second probe is an antibody, single-chain Fv fragment (scFv), antibody fragment-antigen binding (Fab), affimer, or an aptamer. In embodiments, the second probe is an antibody. In embodiments, the second probe is a single-chain Fv fragment (scFv). In embodiments, the second probe is an antibody fragment-antigen binding (Fab). In embodiments, the second probe is an affirmer. In embodiments, the second probe is an aptamer.

In embodiments, the first probe and the second probe are both an antibody. In embodiments, the first probe is a monoclonal antibody and the second probe is a polyclonal antibody. In embodiments, the first probe and the second probe are present at different ratios. For example, the ratio of the first probe and the second probe is 0.75 to 1.0.

In embodiments, the first probe and the second probe are independently a receptor or a ligand-binding portion thereof. In embodiments, the first probe is a receptor or a ligand-binding portion thereof. In embodiments, the second probe is a receptor or a ligand-binding portion thereof. In embodiments, the first probe specifically binds to a receptor or ligand-binding portion thereof. In embodiments, the second probe specifically binds to a receptor or ligand-binding portion thereof. In general, receptors include proteins that transmit a signal in a signaling pathway in response to binding a ligand. Receptors may be intracellular receptors or cell surface receptors. Examples of cell surface receptors include ligand-gated ion channels, G protein-coupled receptors, and receptor tyrosine kinases. Examples of receptors include, without limitation, tyrosine kinase receptor, such as a colony stimulating factor 1 (CSF-1), platelet-derived growth factor (PDGF), epidermal growth factor (EGF), transforming growth factor (TGF), nerve growth factor (NGF), insulin, insulin-like growth factor 1 (IGF-1) receptor, etc.; a G-protein coupled receptor, such as a Gi-coupled, Gq-coupled or Gs-coupled receptor, e.g. a muscarinic receptor (e.g. the subtypes m1, m2, m3, m4, m5), dopamine receptor (e.g. the subtypes D1, D2, D4, D5), opiate receptor (e.g. the subtypes or 6), adrenergic receptor (e.g. the subtypes α1A, α1B, α1C, α2C10, α2C2, α2C4), serotonin receptor, tachykinin receptor, luteinising hormone receptor or thyroid-stimulating hormone receptor, retinoic acid/steroid super family of receptors, mutant forms of receptors such as mutant trk A receptor, mutant EGF receptors, ligand-gated channels including subtypes of nicotinic acetylcholine receptors, GABA receptors, glutamate receptors (NMDA or other subtypes), subtype 3 of the serotonin receptor, and the cAMP-regulated channel. In embodiments, the first probe and the second probe are independently a ligand. In embodiments, the first probe is a ligand. In embodiments, the second probe is a ligand. In general, ligands include proteins that bind to and alter the function of a protein (e.g., an enzyme or a receptor). Ligands may be other proteins, protein fragments, or other molecules. Non-limiting examples of ligands include peptides, polypeptides or proteins, such as cytokines or growth factors. For example, ligands include but are not limited to βc, Cyclophilin A, IL-1, IL-2, IL-3, IL-4, IL-5, IL-6, IL-7, IL-8, IL-10, IL-12, IL-13, G-CSF, M-CSF, GM-CSF, BDNF, CNTF, EGF, EPO, FGF1, FGF2, FGF3, FGF4, FGF5, FGF6, FGF7, FGF8, FGF9, FGF10, FGF11, FGF12, FGF12, FGF13, FGF14, FGF15, FGF16, FGF17, FGF18, FGF19, FGF20, FGF21, FGF22, FGF23, LIF, MCP1, MCP2, KC, MCP3, MCP4, MCP5, M-CSF, MIP1, MIP2, NOF, NT 3, NT4, NT5, NT6, NT7, OSM, PBP, PBSF, PDGF, PECAM-1, PF4, RANTES, SCF, TGFα, TGFβ₁, TGFβ₂, TGFβ₃, TNFα, TNFβ, TPO, VEGF, GH, chemokines, and eotaxin (eotaxin-1, -2 or -3).

In embodiments, the first probe and the second probe are each an antibody. In embodiments, the first probe is an antibody. In embodiments, the second probe is an antibody. In embodiments, specific binding to an antibody would occur under conditions that utilize an antibody that is selected for its specificity for a particular biomolecule. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular biomolecule.

In embodiments, the first probe and the second probe are each an antibody, single-chain Fv fragment (scFV), antibody fragment-antigen binding (Fab), affimer, or an aptamer. In embodiments, the first probe is an antibody, single-chain Fv fragment (scFV), antibody fragment-antigen binding (Fab), affimer, or an aptamer. In embodiments, the second probe is an antibody, single-chain Fv fragment (scFV), antibody fragment-antigen binding (Fab), affimer, or an aptamer. In embodiments, the first probe is a single-chain Fv fragment (scFV), antibody fragment-antigen binding (Fab), affimer, or an aptamer. In embodiments, the second probe is a single-chain Fv fragment (scFV), antibody fragment-antigen binding (Fab), affimer, or an aptamer.

In embodiments, the first probe and the second probe are each an antigen-specific antibody. In embodiments the first probe is an antigen-specific antibody. In embodiments the second probe is an antigen-specific antibody. In embodiments, the first probe and the second probe are each the antigen-binding site (e.g., fragment antigen-binding (Fab) variable region) of an antibody. In embodiments, the antigen-specific antibody is an intact antibody. In embodiments, the intact antibody is a Fab fragment, F(ab′)2 fragment, an Fd fragment, an Fv fragment, a dAb fragment and an isolated CDR. In embodiments, the intact antibody is a Fab fragment. In embodiments, the intact antibody is an F(ab′)2 fragment. In embodiments, the intact antibody is an Fd fragment. In embodiments, the intact antibody is an Fv fragment. In embodiments, the intact antibody is a dAb fragment. In embodiments, the intact antibody is an isolated CDR. In embodiments, the Fab fragment is a monovalent fragment consisting of the VL, VH, CL and CHI domains. In embodiments, the F(ab′)2 fragment is a bivalent fragment comprising two Fab fragments linked by a disulfide bridge at the hinge region. In embodiments, the Fd fragment consists of the VH and CH1 domains. In embodiments, the Fv fragment consists of the VL and VH domains of a single arm of an antibody. In embodiments, the dAb fragment consists of a VH domain. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). A commonly used linker is a 15-residue (Gly4Ser)3 peptide, but other linkers are also known in the art.

In embodiments, the first probe and the second probe are each a monoclonal antibody or polyclonal antibody. In embodiments, the first probe is a monoclonal antibody. In embodiments, the second probe is a monoclonal antibody. In embodiments, the first probe is a polyclonal antibody. In embodiments, the second probe is a polyclonal antibody. In embodiments, the first probe is a monoclonal antibody and the second probe is a polyclonal antibody. In embodiments, the first probe is a polyclonal antibody and the second probe is a monoclonal antibody.

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

In embodiments, the first probe is an oligonucleotide capable of binding to a first biomolecule and the second biomolecule is an oligonucleotide capable of binding to a first target, wherein the first biomolecule and the second biomolecule are different. In embodiments, the probe includes a target hybridization sequence. In embodiments, the target hybridization sequence of each probe is greater than 30 nucleotides. In embodiments, the target hybridization sequence of each probe is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is about 5, about 10, about 15, about 20, about 25, about 30, or about 35 nucleotides in length.

In embodiments, the biomolecule is a nucleic acid sequence, carbohydrate, or protein. In embodiments, the biomolecule is a nucleic acid sequence. In embodiments, the biomolecule is a carbohydrate. In embodiments, the biomolecule is a protein. In embodiments, the biomolecule is a lipid. In embodiments, the biomolecule is a RNA nucleic acid molecule. In embodiments, the biomolecule is a DNA nucleic acid molecule. In embodiments, the first biomolecule and second biomolecule include RNA nucleic acid sequences or DNA nucleic acid sequences. In embodiments, the first biomolecule is a protein and the second biomolecule is a protein. In embodiments, the first biomolecule is different than the second biomolecule (i.e., a different molecule).

In embodiments, the method includes detecting a plurality of biomolecules. In embodiments, the biomolecules are proteins or carbohydrates. In embodiments, the biomolecules are proteins. In embodiments, the biomolecules are carbohydrates. In embodiments, when the biomolecules are proteins and/or carbohydrates, the method includes contacting the proteins with a specific binding reagent, wherein the specific binding reagent includes an oligonucleotide barcode. In embodiments, the specific binding reagent includes an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents in or on the cell. Carbohydrate-specific antibodies are known in the art, see for example Kappler, K., Hennet, T. Genes Immun 21, 224-239 (2020).

In embodiments, the biomolecule is a nucleic acid sequence. In embodiments, the method further includes amplifying the nucleic acid sequence to generate amplification products. In embodiments, the method includes detecting the amplification products.

In embodiments, the first biomolecule and second biomolecule are in a cell. In embodiments, the first biomolecule and second biomolecule are on a cell. In embodiments, the biomolecule is in a cell. In embodiments, the biomolecule is on a cell.

In embodiments, the method includes imaging the immobilized tissue section. In embodiments, the method further includes an imaging modality, immunofluorescence (IF), or immunohistochemistry modality (e.g., immunostaining). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a z-step interval of 1 to 5 μm. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 m, and for image acquisition in the regions of interest, an interval in the range of 2 to 5 μm may be used.

To microscopically visualize tissue sections prepared by the subject methods, in some embodiments the tissue section is embedded in a mounting medium. Mounting medium is typically selected based on its suitability for the reagents used to visualize the cellular biomolecules, the refractive index of the tissue section, and the microscopic analysis to be performed. For example, for phase-contrast work, the refractive index of the mounting medium should be different from the refractive index of the specimen, whereas for bright-field work the refractive indexes should be similar. As another example, for epifluorescence work, a mounting medium should be selected that reduces fading, photobleaching or quenching during microscopy or storage. In certain embodiments, a mounting medium or mounting solution may be selected to enhance or increase the optical clarity of the cleared tissue specimen. Nonlimiting examples of suitable mounting media that may be used include glycerol, CC/Mount™, Fluoromount™ Fluoroshield™, ImmunHistoMount™, Vectashield™, Permount™, Acrytol™, CureMount™ FocusClear™, or equivalents thereof.

The biological targets or molecules to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA. For example, following immobilization on the receiving substrate, the sections may be fixed with methanol, permeabilized with 0.025% Triton in PBS solution, and stained with primary antibodies directed against vimentin (fibroblasts) and macrophages, followed by secondary antibody labeling (e.g., Alexa-594 conjugated secondary antibodies). Additional counterstaining may be performed, for example using 4,6-diamidino-2-phenylindole (DAPI) mounting media to counterstain nuclei.

In an aspect is provided a method of imaging a cell or tissue. In embodiments, the method includes: contacting a cell or tissue with a first probe set, wherein the first probe set includes a plurality of probes, wherein each probe is attached to a first detectable label; binding each probe to a different target of the cell or tissue; imaging (e.g., obtaining an image) the cell or tissue; contacting the cell or tissue with a second probe set, wherein the second probe set includes a plurality of probes, wherein each probe is attached to a second detectable label; binding each probe to a different target of the cell or tissue; and imaging (e.g., obtaining an image) the cell or tissue.

In embodiments, the method includes (i) contacting a cell or tissue with a probe including a detectable label and a quenching moiety, wherein the detectable label is attached to the probe via a cleavable linker; (ii) binding the probe to a target of the cell or tissue; (iii) removing the quenching moiety and detecting the probe bound to a target; (iv) cleaving the cleavable linker; and imaging (e.g., obtaining an image) the cell or tissue. In embodiments, the method includes repeating steps (i) to (iii) to obtain multiple images.

In embodiments, the methods described herein further includes sequencing in situ. In embodiments, the method includes incorporating a target sequence into a circular polynucleotide, amplifying the circular polynucleotide (e.g., amplifying the circular polynucleotide via rolling circle amplification) to generate an amplification product, and sequencing the amplification product. In embodiments, sequencing the amplification product includes identifying or determining the target sequence (or a complement thereof). Known techniques for incorporating a target sequence into a circular polynucleotide include methods and compositions described, for example, in U.S. Pat. Nos. 11,434,525, 11,680,288, 11,753,678, and 12,006,534, each of which are incorporated herein by reference.

Briefly, in an aspect is provided a method of detecting a nucleic acid sequence in a cell or tissue. In embodiments, the method further includes detecting one or more biomolecules in the cell or tissue, for example, one or more organelles. In embodiments, detecting includes sequencing in a cell or tissue. In embodiments, the method includes detecting a synthetic sequence (e.g., a sequence introduced into the cell via genome editing technique such as CRISPR).

In embodiments, the method includes binding a polynucleotide probe to a nucleic acid molecule in the cell or tissue and incorporating a sequence of the nucleic acid molecule into the polynucleotide probe; amplifying the polynucleotide probe to form a first amplification product; and binding a first fluorescently labeled nucleotide to the amplification product. In embodiments, binding a fluorescently labeled nucleotide includes hybridizing a primer to the amplification product and incorporating the fluorescently labeled nucleotide. In embodiments, the method includes incorporating a plurality of fluorescently labeled nucleotides into the primer, wherein an emission light is detected and a reversible terminator (e.g., a labelled, reversibly terminated nucleotide) is removed prior to the incorporation of the next nucleotide.

In embodiments, the method includes hybridizing a first hybridization sequence of the polynucleotide probe to a first sequence of the first nucleic acid molecule, and hybridizing a second hybridization sequence of the polynucleotide probe to a second sequence of the nucleic acid molecule, wherein the nucleic acid molecule includes a target sequence between the first sequence and the second sequence and extending the polynucleotide probe along the target sequence to generate a complement of the target sequence, and ligating the complement of the target sequence to the polynucleotide probe thereby forming a circular oligonucleotide.

In embodiments, the method includes sequencing a plurality of target nucleic acids of a cell in situ. In embodiments, the method includes the following steps in situ for each of the plurality of target nucleic acids: i) hybridizing an oligonucleotide primer to the target nucleic acid, wherein the oligonucleotide primer includes a first region at a 3′ end that hybridizes to a first complementary region of the target nucleic acid, and a second region at a 5′ end that hybridizes to a second complementary region of the target nucleic acid, wherein the second complementary region is 5′ with respect to the first complementary region; ii) circularizing the oligonucleotide primer to generate a circular oligonucleotide, wherein circularizing includes extending the 3′ end of the oligonucleotide primer (e.g., extending the 3′ end of the primer using a polymerase to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer; iii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iv) sequencing the extension product of step (iii).

In embodiments, amplifying the circular polynucleotide generates an amplification product including multiple copies of the target sequence, or a complement thereof. In embodiments, the method includes serially cycling through detection cycles to determine the sequence (e.g., the order of the nucleotides) of the target sequence), wherein each detection cycle includes hybridizing, detecting, and removing a fluorescently labelled oligonucleotide. In embodiments, detecting includes sequencing the amplification product (e.g., using a sequencing by synthesis or sequencing by binding process).

In embodiments, forming the circular polynucleotide includes ligating a first end and a second end of the probe oligonucleotide together. In embodiments, ligating includes forming a covalent bond from the first end and the second end. As those of skill in the art appreciate, two nucleotide sequences that that are to ligated together will generally directly abut one another.

In embodiments, the method includes contacting a cell or tissue with a polynucleotide probe and hybridizing a first hybridization sequence of the polynucleotide probe to a first target sequence of the RNA or DNA molecule, and hybridizing a second hybridization sequence of the polynucleotide probe to a second target sequence of the RNA or DNA molecule, wherein the RNA or DNA molecule comprises a target sequence between the first target sequence and the second target sequence; extending the polynucleotide probe along the target sequence to generate a complement of the target sequence, and ligating the complement of the target sequence to the polynucleotide probe thereby forming a circular oligonucleotide; extending an amplification primer hybridized to the circular oligonucleotide with a polymerase to generate an extension product comprising the target sequence; and hybridizing a sequencing primer to the extension product and sequencing the target sequence in the cell or tissue, thereby detecting the RNA or DNA molecule. In embodiments, the nucleic acid molecule is cDNA. In embodiments, the nucleic acid molecule is DNA. In embodiments, the nucleic acid molecule is RNA.

In embodiments, the target is an RNA nucleic acid sequence or DNA nucleic acid sequence. In embodiments, the target is an RNA nucleic acid sequence or DNA nucleic acid sequence from the same cell. In embodiments, the target is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments, the target is mRNA, tRNA (transfer RNA), rRNA (ribosomal RNA), or noncoding RNA (such as lncRNA (long noncoding RNA)). In embodiments, the targets are on different regions of the same RNA nucleic acid sequence. In embodiments, the targets are cDNA target nucleic acid sequences and before step i), the RNA nucleic acid sequences are reverse transcribed to generate the cDNA target nucleic acid sequences. In embodiments, reverse transcription of the RNA nucleic acid is performed with a reverse transcriptase, for example, Tth DNA polymerase or mutants thereof. In embodiments, the targets are not reverse transcribed to cDNA, i.e., the oligonucleotide primer is hybridized directly to the target nucleic acid.

In embodiments, the methods and compositions described herein are utilized to analyze the various sequences of TCRs and BCRs from immune cells, for example various clonotypes. In embodiments, the target nucleic acid includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the entire sequence of the target is about 1 to 3 kb, and only a portion of that target (e.g., 50 to 100 nucleotides) is sequenced. In embodiments, the target is about 1 to 3 kb. In embodiments, the target is about 1 to 2 kb. In embodiments, the target is about 1 kb. In embodiments, the target is about 2 kb. In embodiments, the target is less than 1 kb. In embodiments, the target is about 500 nucleotides. In embodiments, the target is about 200 nucleotides. In embodiments, the target is about 100 nucleotides. In embodiments, the target is less than 100 nucleotides. In embodiments, the target is about 5 to 50 nucleotides.

In embodiments the target is an RNA transcript. In embodiments the target is a single stranded RNA nucleic acid sequence. In embodiments, the target is an RNA nucleic acid sequence or a DNA nucleic acid sequence (e.g., cDNA). In embodiments, the target is a cDNA target nucleic acid sequence and before step i), the RNA nucleic acid sequence is reverse transcribed to generate the cDNA target nucleic acid sequence. In embodiments, reverse transcription of the RNA nucleic acid is performed with a reverse transcriptase, for example, Tth DNA polymerase or mutants thereof. In embodiments, the target is genomic DNA (gDNA), mitochondrial DNA, chloroplast DNA, episomal DNA, viral DNA, or copy DNA (cDNA). In embodiments, the target is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target is a cancer-associated gene. In embodiments, to minimize amplification errors or bias, the target is not reverse transcribed to generate cDNA.

In embodiments, the polynucleotide probe includes DNA. In embodiments, the polynucleotide probe consists of DNA. In embodiments, the polynucleotide probe is a single-stranded polynucleotide comprising at least one amplification primer binding sequence, at least one sequencing primer binding sequence, or both one amplification primer binding sequence and one sequencing primer binding sequence. In embodiments, the first hybridization sequence, the second hybridization sequence, or both the first and second hybridization sequences of the polynucleotide probe comprise one or more locked nucleic acid (LNA) nucleotides. In embodiments, an amplification primer and a sequencing primer are complementary to different primer binding sequences. In embodiments, the primer binding sequence is complementary to a fluorescent in situ hybridization (FISH) probe. FISH probes may be custom designed using known techniques in the art, see for example Gelali, E., Girelli, G., Matsumoto, M. et al. Nat Commun 10, 1636 (2019).

In embodiments, the target sequence is 1 to about 15 nucleotides. In embodiments, the target sequence is 1 to about 25 nucleotides. In embodiments, the target sequence is 10 to about 25 nucleotides. In embodiments, the target sequence is 5 to about 15 nucleotides.

In embodiments, the method includes circularizing and ligating the complementary sequence to the 5′ end of the polynucleotide probe. In embodiments, circularizing the oligonucleotide primer to generate a circular oligonucleotide includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer. In embodiments, the ligation includes enzymatic ligation. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR® ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof.

In embodiments, amplifying the circular polynucleotide includes hybridizing a primer to the circular polynucleotide and extending the primer with a strand-displacing polymerase. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the method includes contacting the target with an amplification primer and oligonucleotide primer in the same reaction (e.g., simultaneously). In embodiments, the method includes fixing the amplification products (e.g., contacting the amplification product with formalin).

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g., a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, amplifying includes incubating the circular polynucleotide with a strand-displacing polymerase for about 15 minutes to about 2 hours. In embodiments, amplifying includes incubating the circular polynucleotide with a strand-displacing polymerase for about 30 minutes to about 60 minutes. In embodiments, amplifying includes binding an amplification primer to the primer binding sequence and extending the amplification primer with a strand-displacing polymerase.

In embodiments, the probe oligonucleotide further includes a primer binding sequence. For example, a primer binding sequence includes a nucleic acid sequence of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

In embodiments, the method further includes detecting the amplification products. In embodiments, detecting includes binding a detection agent (e.g., a labeled probe) to the amplification product. In embodiments, the detection agent includes a fluorescently labeled probe. In embodiments, the method includes exciting and detecting the label. In embodiments, detecting includes serially contacting the amplification products with labeled probes (e.g., labeled oligonucleotides or labeled nucleotides).

The phrase “labeled probes” refers to mixture of nucleic acids that are detectably labeled, e.g., fluorescently labeled, such that the presence of the probe, as well as, any target sequence to which the probe is bound can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety. In embodiments, the method includes hybridizing a primer to the amplification product and incorporating a labeled nucleotide into the primer.

In embodiments, sequencing includes hybridizing a sequencing primer to the amplification product and incorporating one or more labeled nucleotides, and detecting the incorporated one or more labeled nucleotides so as to identify the sequence.

In embodiments, the method includes sequencing the extension products, which includes the target nucleic acid sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, the method includes sequencing the amplification products (e.g., a plurality of different amplification products). In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP or acyclic nucleotide) into the first sequencing primer.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon T K. Error Correction Coding: Mathematical Methods and Algorithms. ed. 1st Wiley: 2005., each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) or acyclic nucleotide triphosphates to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) or acyclic nucleotide triphosphates to prevent further extension of the sequencing read product.

In embodiments, sequencing includes sequencing by synthesis, sequencing by binding, or sequencing by ligation. In embodiments, sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the amplification product.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In an aspect is provided a method of identifying a cell that includes a synthetic target. In embodiments, the method includes detecting whether a synthetic target is present in the cell by detecting a plurality of different targets within an optically resolved volume of a cell in situ, according to the methods described herein, including embodiments, and identifying a cell that includes a synthetic target when the presence of the synthetic target is detected in the cell.

In embodiments, the method includes sequencing the synthetic target sequence in situ. In embodiments, the method includes incorporating the synthetic target sequence into a circular polynucleotide, amplifying the circular polynucleotide (e.g., amplifying the circular polynucleotide via rolling circle amplification) to generate an amplification product, and sequencing the amplification product. In embodiments, sequencing the amplification product includes identifying or determining the synthetic target sequence (or a complement thereof). Known techniques for incorporating a target sequence into a circular polynucleotide include methods and compositions described, for example, in U.S. Pat. Nos. 11,434,525, 11,680,288, 11,753,678, and 12,006,534. In embodiments, the method further includes detecting one or more biomolecules in the cell or tissue, for example, one or more organelles.

In embodiments, the method includes binding a polynucleotide probe to a nucleic acid molecule (e.g., the nucleic acid molecule that includes the synthetic target) in the cell or tissue and incorporating a sequence of the nucleic acid molecule into the polynucleotide probe; amplifying the polynucleotide probe to form a first amplification product; and binding a first fluorescently labeled nucleotide to the amplification product. In embodiments, binding a fluorescently labeled nucleotide includes hybridizing a primer to the amplification product and incorporating the fluorescently labeled nucleotide. In embodiments, the method includes incorporating a plurality of fluorescently labeled nucleotides into the primer, wherein an emission light is detected and a reversible terminator (e.g., a labelled, reversibly terminated nucleotide) is removed prior to the incorporation of the next nucleotide.

In embodiments, the method includes the following steps in situ for each of the plurality of target nucleic acids: i) hybridizing an oligonucleotide primer to the target nucleic acid, wherein the oligonucleotide primer includes a first region at a 3′ end that hybridizes to a first complementary region of the target nucleic acid, and a second region at a 5′ end that hybridizes to a second complementary region of the target nucleic acid, wherein the second complementary region is 5′ with respect to the first complementary region; ii) circularizing the oligonucleotide primer to generate a circular oligonucleotide, wherein circularizing includes extending the 3′ end of the oligonucleotide primer (e.g., extending the 3′ end of the primer using a polymerase to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer; iii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iv) sequencing the extension product of step (iii).

In embodiments the synthetic target is a chimeric antigen receptor (CAR) or a gene that encodes a chimeric antigen receptor (CAR). In embodiments the synthetic target is a target introduced to the cell by genetic engineering methods (e.g., transcription activator-like effector nucleases (TALENs) or clustered regularly interspaced short palindromic repeats (CRISPR) methods).

### III. Compositions, Devices, & Kits

In an aspect is provided a cell or tissue including two target complexes, as described herein. In embodiments, the a first target complex includes a first probe bound to a first biomolecule, wherein the first probe includes a first detectable label attached to the first probe via a first linker; and the second target complex includes a second probe bound to a second biomolecule, wherein the second probe includes a second detectable label and a quenching moiety, wherein the second detectable label is attached to the second probe via a second linker; and the quenching moiety is attached to the second probe via a cleavable linker.

In embodiments, the first probe and the second probe are each an agent capable of selectively binding a protein. In embodiments, the first probe and the second probe each specifically bind a particular protein (e.g., protein antigen or epitope). In embodiments, the first probe and the second probe are an immunoglobulin. In embodiments, the first probe is an immunoglobulin. In embodiments, the second probe is an immunoglobulin. In embodiments, the immunoglobulin is IgA, IgD, IgE, IgG, or IgM. In embodiments, the immunoglobulin is IgA. In embodiments, the immunoglobulin is IgD. In embodiments, the immunoglobulin is IgE. In embodiments, the immunoglobulin is IgG. In embodiments, the immunoglobulin is IgM.

In embodiments, the first probe is capable of selectively binding a carbohydrate. In embodiments, the first probe is capable of selectively binding a lipid. In embodiments, the first probe is capable of selectively binding a nucleic acid. In embodiments, the first probe is capable of selectively binding a subcellular organelle. In embodiments, the first probe is capable of selectively binding an apoptotic cell. In embodiments, the second probe is capable of selectively binding a carbohydrate. In embodiments, the second probe is capable of selectively binding a lipid. In embodiments, the second probe is capable of selectively binding a nucleic acid. In embodiments, the second probe is capable of selectively binding a subcellular organelle. In embodiments, the second probe is capable of selectively binding an apoptotic cell. In embodiments, the probe described herein is capable of selectively binding a carbohydrate. In embodiments, the probe described herein is capable of selectively binding a lipid. In embodiments, the probe described herein is capable of selectively binding a nucleic acid. In embodiments, the probe described herein is capable of selectively binding a subcellular organelle. In embodiments, the probe described herein is capable of selectively binding an apoptotic cell.

In embodiments, the first probe is a compound having the formula: Ab1-L¹-Dye1; and wherein the second probe is a compound having the formula: Ab2-L²-Dye2-L¹-Q¹; wherein Ab1 is a first antibody and Ab2 is a second antibody; L¹ and L² are orthogonally cleavable linkers; Dye1 and Dye2 are independently fluorescent moieties; Q¹ is a quenching moiety. In embodiments, a cleavable linker is specifically cleavable in response to external stimuli.

In embodiments, the first probe is a compound having the formula: OL1-L¹-Dye1; and wherein the second probe is a compound having the formula: OL2-L²-Dye2-L¹-Q¹; wherein OL1 is a first oligonucleotide probe and OL2 is a second oligonucleotide probe; L¹ and L² are orthogonally cleavable linkers; Dye1 and Dye2 are independently fluorescent moieties; Q¹ is a quenching moiety. For example, oligonucleotide probes using the formulae: OL1-L¹-Dye1 for the first oligonucleotide probe and OL2-L²-Dye2-L¹-Q¹ are depicted in FIG. 2B. In embodiments, a cleavable linker is specifically cleavable in response to external stimuli.

In embodiments, the first probe is a compound having the formula: Pr1-L¹-Dye1; and wherein the second probe is a compound having the formula: Pr2-L²-Dye2-L¹-Q¹; wherein Pr1 is a first probe targeting a first biomolecule described herein and Pr2 is a second probe targeting a second biomolecule described herein; L¹ and L² are orthogonally cleavable linkers; Dye1 and Dye2 are independently fluorescent moieties; Q¹ is a quenching moiety.

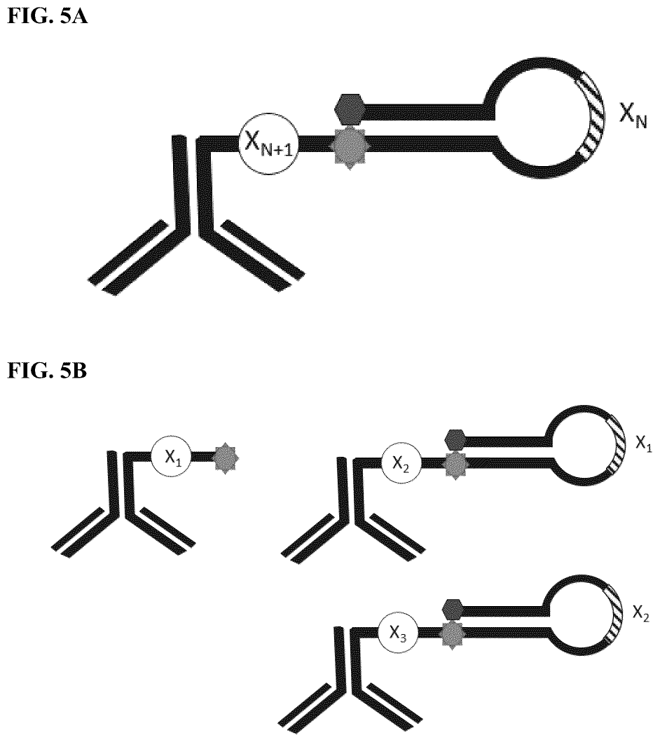

In embodiments, the probe is a molecular beacon probe. In embodiments, the first probe includes first antibody attached to a detectable label via a first cleavable linker, and the second probe is an antibody molecular beacon, wherein the second probe includes a second antibody attached to the molecular beacon with a detectable label via a second cleavable linker and a quenching moiety via a first cleavable linker (as illustrated in FIG. 5B). As used herein, the term “molecular beacon” is used in accordance with its plain and ordinary meaning and refers an oligonucleotide hybridization probe with a hairpin conformation that is attached to both a fluorescent moiety and quenching moiety. Typically, a molecular beacon contains a single stranded oligonucleotide loop region, a double stranded stem region, a fluorescent moiety covalently attached to the 5′ end, and a quenching moiety covalently attached to the 3′ end. In absence of target nucleotide sequences capable of hybridizing with the complementary sequences within the molecular beacon, the fluorescent moiety on the 5′ end of the molecular beacon is brought into proximity to the quenching moiety on the 3′ end and therefore, the fluorescent moiety is internally quenched. Upon hybridizing with the target sequence, the hairpin dissociates and thus, liberating the fluorescent moiety from the quenching moiety (see e.g., Bhattacharyya, S. and Ducheyne, P. (2011). Intracellular Probes. Comprehensive Biomaterials, 447-470). In embodiments, the single stranded oligonucleotide loop region includes 15-30 nucleotides, 20-25 nucleotides, or 13-19 nucleotides. In embodiments, the single stranded oligonucleotide loop region includes a cleavable site. In embodiments, the double stranded stem region includes 5-8 nucleotides, 4-6 nucleotides, 4 or less nucleotides. In embodiments, the fluorescent moiety that is attached 5′ end of the molecular beacon is attached to the Fc region of an antibody via a cleavable linker.

In an aspect is provided a solid support including a first probe attached to the solid support; an analyte (e.g., biomolecule) attached to the first probe; a second probe attached to the analyte, wherein the first probe and the second probe are probes as described herein. In embodiments, the solid support is a multiwell container. In embodiments, the solid support is a flow cell. In embodiments, the solid support is the surface of a flow cell. In embodiments, the solid support is a polymer coated surface of a flow cell. In embodiments, the flow cell is a closed flow cell including fluid inlets and outlets, and a sample chamber or compartment that is not open to the surrounding environment. In embodiments, the flow cell is an open flow cell including fluid inlets and outlets, and a sample chamber or compartment that is open to and/or accessible from the surrounding environment. In embodiments, the flow cell is fabricated from any of a variety of materials known to those of skill in the art including glass (e.g., borosilicate glass, soda lime glass, etc.), fused silica (quartz), silicon, polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), organic modified ceramic (e.g., Ormocomp®), cyclic olefin copolymers (COC), polyethylene terephthalate (PET), polydimethylsiloxane (PDMS), etc.), polyetherimide (PEI) and/or perfluoroelastomer (FFKM) or any combination thereof. In embodiments, the flow cell is optically transparent to facilitate use with spectroscopic or imaging-based detection techniques. In embodiments, the entire flow cell will be optically transparent. Alternatively, in embodiments, only a portion of the flow cell (e.g., an optically transparent “window”) will be optically transparent.

In embodiments, the wells of the multiwell container are separated from each other by about 1 mm to about 10 mm. In embodiments, the wells of the multiwell container are separated from each other by about 2 mm. In embodiments, the wells of the multiwell container are separated from each other by about 3 mm. In embodiments, the wells of the multiwell container are separated from each other by about 4 mm. In embodiments, the wells of the multiwell container are separated from each other by about 5 mm. In embodiments, the wells of the multiwell container are separated from each other by about 6 mm. In embodiments, the wells of the multiwell container are separated from each other by about 7 mm. In embodiments, the wells of the multiwell container are separated from each other by about 8 mm. In embodiments, the wells of the multiwell container are separated from each other by about 9 mm. It is also understood that the separation of the wells of the multiwell container will ultimately depend on the systems and/or apparatus used to analyze later reactions.

In embodiments, the wells of the multiwell container are separated from each other by about 0.2 μm to about 2.0 μm. In embodiments, the wells of the multiwell container are separated from each other by about 0.3 μm. In embodiments, the wells of the multiwell container are separated from each other by about 0.4 μm. In embodiments, the wells of the multiwell container are separated from each other by about 0.5 μm. In embodiments, the wells of the multiwell container are separated from each other by about 0.6 μm. In embodiments, the wells of the multiwell container are separated from each other by about 0.7 μm. In embodiments, the wells of the multiwell container are separated from each other by about 0.8 μm. In embodiments, the wells of the multiwell container are separated from each other by about 0.9 μm. In embodiments, the wells of the multiwell container are separated from each other by about 1.0 μm. In embodiments, the wells of the multiwell container are separated from each other by about 1.1 μm. In embodiments, the wells of the multiwell container are separated from each other by about 1.2 μm. In embodiments, the wells of the multiwell container are separated from each other by about 1.3 μm. In embodiments, the wells of the multiwell container are separated from each other by about 1.4 μm. In embodiments, the wells of the multiwell container are separated from each other by about 1.5 μm. In embodiments, the wells of the multiwell container are separated from each other by about 1.6 μm. In embodiments, the wells of the multiwell container are separated from each other by about 1.7 μm. In embodiments, the wells of the multiwell container are separated from each other by about 1.8 μm. In embodiments, the wells of the multiwell container are separated from each other by about 1.9 μm. It is also understood that the separation of the wells of the multiwell container will ultimately depend on the systems and/or apparatus used to analyze later reactions. In embodiments, each particle is bound to a discrete site on a substrate, wherein each discrete site of the substrate is separated by an interstitial region.

In embodiments, the well contains a gel. In embodiments, the gel has a colloidal structure. In embodiments the gel is agarose; polymer mesh structure, such as gelatin; or cross-linked polymer structure, such as polyacrylamide or a derivative thereof. In embodiments, analytes, such as polynucleotides, can be attached to the gel via covalent or non-covalent means. Exemplary methods and reactants for attaching nucleic acids to gels are described, for example, in US 2011/0059865, which is incorporated herein by reference. In embodiments the analytes, sample, tissue, or cell can include nucleic acids and the nucleic acids can attach to the gel or polymer via their 3′ oxygen, 5′ oxygen, or at other locations along their length such as via a base moiety of the 3′ terminal nucleotide, a base moiety of the 5′ nucleotide, and/or one or more base moieties elsewhere in the molecule.

The wells may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern. A regular array of such regions is advantageous for detection and data analysis of signals collected from the arrays during an analysis.

In embodiments, the well is substantially square. In embodiments, the well is square. In embodiments, the well is F-bottom. In embodiments, the wells are substantially round flat bottom wells. In embodiments, the well is C-bottom. In embodiments, the well is V-bottom. In embodiments, the well is U-bottom.

In embodiments, the multiwell container includes 24 substantially round flat bottom wells. In embodiments, the multiwell container includes 48 substantially round flat bottom wells. In embodiments, the multiwell container includes 96 substantially round flat bottom wells. In embodiments, the multiwell container includes 384 substantially round flat bottom wells. In embodiments, the multiwell container includes 24 substantially square flat bottom wells. In embodiments, the multiwell container includes 48 substantially square flat bottom wells. In embodiments, the multiwell container includes 96 substantially square flat bottom wells. In embodiments, the multiwell container includes 384 substantially square flat bottom wells.

The solid supports for some embodiments have at least one surface located within a flow cell. In embodiments, the flow cell includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate.

In embodiments, the flow cell includes one or more channel(s) bored onto the glass substrate. In embodiments, the flow cell includes a channel bored into the glass substrate. In embodiments, the flow cell includes a plurality of channels bored into the glass substrate. In embodiments, the flow cell includes 2 channels bored into the glass substrate. In embodiments, the flow cell includes 3channels bored into the glass substrate. In embodiments, the flow cell includes 4 channels bored into the glass substrate. In embodiments, the width of the channel is from about 1 to 5 mm. In embodiments, the width of the channel is from about 5 to 10 mm. In embodiments, the width of the channel is from about 10 to 15 mm. In embodiments, the width of the channel is from about 5 mm. In embodiments, the width of the channel is from about 11 mm.

In embodiments, the biomolecule is bound to both the first probe and second probe thereby forming a complex. In embodiments, the biomolecule is bound to the first probe, thereby forming a complex. In embodiments, the biomolecule is bound to the second probe, thereby forming a complex. In embodiments, the biomolecule described herein is bound to a probe described herein, thereby forming a complex. In embodiments, the biomolecule may be bound to the first probe through non-covalent or covalent means (e.g., following complex formation, first probe may be crosslinked to the biomolecule). In embodiments, the biomolecule may be bound to the second probe through non-covalent or covalent means (e.g., following complex formation, the second probe may be crosslinked to the biomolecule). In embodiments, the first probe and the second probe are cross-linked. Crosslinking is the process of joining two or more molecules, such as by a covalent bond, non-covalent interactions, or interactions with one or more intermediate molecules. Examples of crosslinking reagents (or crosslinkers) include molecules that contain two or more reactive ends capable of chemically attaching to specific functional groups (e.g., primary amines, sulfhydryls, etc.) on proteins or other molecules. The crosslinking may be direct, for example, a covalent bond. The cross-linking may be indirect, for example, a complex of antibodies that join the first probe to the second probe (which may optionally be stabilized by further cross-linking). By way of example, the complex of antibodies may include anti-mouse antibody binding moieties and/or Fc antibodies. Various cross-linking reagents and processes are available, particularly for cross-linking proteins. Examples of some common crosslinkers are the imidoester crosslinker dimethyl suberimidate, the N-Hydroxysuccinimide-ester crosslinker BS3 and formaldehyde. Each of these crosslinkers induces nucleophilic attack of the amino group of lysine and subsequent covalent bonding via the crosslinker. The zero-length carbodiimide crosslinker EDC functions by converting carboxyls into amine-reactive isourea intermediates that bind to lysine residues or other available primary amines. SMCC or its water-soluble analog, Sulfo-SMCC, is commonly used to prepare antibody-hapten conjugates for antibody development. An in-vitro cross-linking method, termed PICUP (photo-induced cross-linking of unmodified proteins) is a process in which ammonium persulfate (APS), which acts as an electron acceptor, and tris-bipyridylruthenium (II) cation ([Ru(bpy)₃]²⁺) are added to the protein of interest and irradiated with UV light. In-vivo crosslinking of protein complexes using photo-reactive amino acid analogs is a method in which cells are grown with photoreactive diazirine analogs to leucine and methionine, which are incorporated into proteins. Upon exposure to ultraviolet light, the diazirines are activated and bind to interacting proteins that are within a few Angstroms of the photo-reactive amino acid analog (UV cross-linking).

In an aspect is provided a cell or tissue section including a cell, wherein the cell includes: a first organelle bound to a first probe, wherein the first probe includes a specific binding agent bound to the first organelle and a fluorescent dye linked to the specific binding agent via a first cleavable linker; a second organelle bound to a second probe, wherein the second probe includes a specific binding agent bound to the second organelle and a fluorescent dye linked to the specific binding agent via a second cleavable linker; and a fluorescent stain bound to a nucleic acid molecule. In embodiments, the tissue section further includes a third organelle bound to a third probe, wherein the third probe includes a specific binding agent bound to the third organelle and a linker remnant covalently attached to the specific binding agent. In embodiments, the linker remnant is a portion of the cleavable linker that remains attached to the specific binding agent following cleavage.

In an aspect is provided a nucleic acid polymerase complex including a nucleic acid polymerase, wherein the nucleic acid polymerase is bound to a compound as described herein. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044).

In another aspect is provided a kit, including: (i) a first compound having the formula: Ab1-L¹-Dye1; (ii) a second compound having the formula: Ab2-L²-Dye2-L¹-Q¹; (iii) a third compound having the formula: Ab3-L³-Dye3-L²-Q²; wherein, Ab1 is a first antibody; Ab2 is a second antibody; Ab3 is a third antibody; L¹, L² and L³ are orthogonally cleavable linkers; Dye1, Dye2, and Dye3 are independently fluorescent moieties; Q¹ and Q² are quenching moieties, wherein Dye2 and Q1 are a first fluorescent-quencher pair; and Dye3 and Q2 are a second fluorescent-quencher pair. In embodiments, the kit further includes a photodamage mitigating agent. Non-limiting examples of a photodamage mitigating agent include ascorbic acid, dithiothreitol (DTT), mercaptoethylamine (MEA), P-mercaptoethanol (BME), N-propyl gallate, p-phenylenediamene (PPD), hydroquinone, sodium azide (NaN₃), diazobicyclooctane (DABCO), cyclooctatetraene (COT), Trolox and its derivatives, butylated hydroxytoluene (BHT), ergothioneine, methionine, cysteine, beta-dimethyl cysteine, histidine, tryptophan, mercaptopropionylglycine, MESNA, glutathione, N-acetyl cysteine, captopril, lycopene, gamma-carotene, astazanthin, canthazanthin, alpha-carotene, beta-carotene, gamma-carotene, bixin, zeaxanthin, lutein, bilirubin, biliverdin, tocopherols, polyene dialdehydes, 32 melatonin, octocopheryl succinate and its analogs, pyridoxinel and its derivatives, hydrazine, sodium sulfite, and hydroxylamine. In embodiments, the photodamage mitigating agent is sodium pyruvate, N,N′-dimethylthiourea, mannitol, DMSO, carboxy-PTIO, 6-hydroxy-2,5,7,8-tetramethylchroman-2-carboxylic acid, alpha-tocopherol, 2-phenyl-1,2,benzisoselenazol-3(2H)-one, uric acid, sodium azide, or manganese(III)-tetrakis(4-benzoic acid) porphyrin, 4,5-dihydroxybenzene-1,3-disulfonate. In embodiments, the photodamage mitigating agent is 3-carboxy-proxyl, N-propyl gallate, ascorbic acid, methyl viologen, Trolox, or Trolox-quinone.

In another aspect is provided a kit, including: (i) a first compound having the formula: OL1-L¹-Dye1; (ii) a second compound having the formula: OL2-L²-Dye2-L¹-Q¹; (iii) a third compound having the formula: OL3-L³-Dye3-L²-Q²; wherein, OL1 is a first oligonucleotide probe; OL2 is a second oligonucleotide probe; OL3 is a third oligonucleotide probe; L¹, L² and L³ are orthogonally cleavable linkers; Dye1, Dye2, and Dye3 are independently fluorescent moieties; Q¹ and Q² are quenching moieties, wherein Dye2 and Q1 are a first fluorescent-quencher pair; and Dye3 and Q2 are a second fluorescent-quencher pair. In embodiments, the kit further includes a photodamage mitigating agent.

In another aspect is provided a kit, including: (i) a first compound having the formula: Pr1-L¹-Dye1; (ii) a second compound having the formula: Pr2-L²-Dye2-L¹-Q¹; (iii) a third compound having the formula: Pr3-L³-Dye3-L²-Q²; wherein, Pr1 is a first probe targeting a first biomolecule described herein; Pr2 is a second probe targeting a second biomolecule described herein; Pr3 is a third probe targeting a third biomolecule described herein; L¹, L² and L³ are orthogonally cleavable linkers; Dye1, Dye2, and Dye3 are independently fluorescent moieties; Q¹ and Q² are quenching moieties, wherein Dye2 and Q1 are a first fluorescent-quencher pair; and Dye3 and Q2 are a second fluorescent-quencher pair. In embodiments, the kit further includes a photodamage mitigating agent.

In embodiments, a fluorescent-quencher pair refers to a fluorophore including an emission spectrum and a quenching moiety including an absorption spectrum, wherein the absorption spectrum overlaps with the emission spectrum. In embodiments, the fluorescent-quencher pair is a FRET pair of detectable moieties. In embodiments, Dye2 and Q1 are a FRET pair, where Dye2 acts as a FRET donor and Q1 acts as a FRET acceptor. In embodiments, Dye3 and Q2 are a FRET pair, where Dye3 acts as a FRET donor and Q2 acts as a FRET acceptor. In embodiments, the wavelength of light emitted from the FRET acceptor is isolated using an emission filter (e.g., a band pass filter) and detected by the detector. In embodiments, the wavelength of light emitted from the FRET acceptor is filtered out via an emission filter (e.g., a band pass filter) and is undetected. The terms “emission filter” and “band pass filter” are used in accordance with its plain and ordinary meaning and refers optical filters used to transmit desired wavelengths to the detecting module within spectroscopic instruments. As appreciated by one of skill in the art, the FRET acceptor could be a nonfluorescent FRET acceptor, and upon absorbing energy emitted from the FRET donor, the nonfluorescent FRET acceptor emits the absorbed energy as heat, as described by Lakowicz (see Mechanisms and Dynamics of Fluorescence Quenching. (2006). Principles of Fluorescence Spectroscopy, 331-351). In embodiments, Dye2 and Q1 are a FRET pair, where Dye2 acts as a FRET donor and Q1 acts as a nonfluorescent FRET acceptor. In embodiments, Dye3 and Q2 are a FRET pair, where Dye3 acts as a FRET donor and Q2 acts as a nonfluorescent FRET acceptor.

In embodiments, Dye1, Dye2, and Dye3 are each independently a fluorescent dye moiety, for example, a rhodamine moiety, a sulforhodamine 101 moiety, a fluorescein moiety, a cyanine moiety, an indocyanine green moiety, a triarylmethane, or a coumarin moiety. A fluorescent dye moiety may be understood in the broadest sense as any dye moiety enabling fluorescence detection. Preferably, such fluorescence detection is in a range of from 400 to 1000 nm, i.e., in the visible spectrum and in the Near Infrared (NIR) spectrum, in particular in a range of from 400 to 800 nm, i.e., in the visible spectrum. Additionally, or alternatively, the dye moiety may also be chromatic, i.e., provoke a color perception when illuminated by any light. Such chromatic effect may be provoked by absorbing light of one or more particular wavelength range(s) in the visible range (i.e., in range(s) from approximately 400 nm to approximately 800 nm) and/or by emitting light of one or more particular wavelength range(s) in the visible range. Examples of detectable labels or derivatives include Cy®3 (e.g., a detectable label derived from Cy®3) and Cy®5 (e.g., a detectable label derived from Cy®5), fluorescein (e.g., a detectable label derived from fluorescein) and tetramethylrhodamine (e.g., a detectable label derived from tetramethylrhodamine), 5-((2-acetamidoethyl)amino)naphthalene-1-sulfonic acid (e.g., a detectable label derived from 5-((2-acetamidoethyl)amino)naphthalene-1-sulfonic acid) or 5-((2-Aminoethyl)amino)naphthalene-1-sulfonic acid (e.g., a detectable label derived from 5-((2-Aminoethyl)amino)naphthalene-1-sulfonic acid) and fluorescein (e.g., a detectable label derived from fluorescein), 5-((2-acetamidoethyl)amino)naphthalene-1-sulfonic acid (e.g., a detectable label derived from 5-((2-acetamidoethyl)amino)naphthalene-1-sulfonic acid) or 5-((2-Aminoethyl)amino)naphthalene-1-sulfonic acid (e.g., a detectable label derived from 5-((2-Aminoethyl)amino)naphthalene-1-sulfonic acid) and (E)-4-((4-(dimethylamino)phenyl)diazenyl)benzoic acid (e.g., a detectable label derived from (E)-4-((4-(dimethylamino)phenyl)diazenyl)benzoic acid), fluorescein (e.g., a detectable label derived from fluorescein) and fluorescein (e.g., a detectable label derived from fluorescein), BODIPY® (e.g., a detectable label derived from BODIPY®) and BODIPY® (e.g., a detectable label derived from BODIPY®), fluorescein (e.g., a detectable label derived from fluorescein) and QSY™7 (e.g., a detectable label derived from QSY™7) and QSY™9 (e.g., a detectable label derived from QSY9™), various combinations of Alexa Fluor dyes (e.g., Alexa Fluor® 488-Alexa Fluor®555, and detectable labels derived therefrom), or various combinations of ATTO® dyes (e.g., Atto® 488-Atto® 532, and detectable labels derived therefrom). In embodiments, Dye1, Dye2, and Dye3 each include the same fluorescent moiety. In embodiments, Dye 1, Dye2, and Dye3 are spectrally distinct fluorescent moieties. In embodiments, Dye1, Dye2, and Dye3 each generate the same maximum emission wavelength. In embodiments, Dye1, Dye2, and Dye3 each include different fluorescent moieties.

In embodiments, Dye1 is a rhodamine moiety, a sulforhodamine 101 moiety, a fluorescein moiety, a cyanine moiety, an indocyanine green moiety, a triarylmethane, or a coumarin moiety. In embodiments, Dye2 is a rhodamine moiety, a sulforhodamine 101 moiety, a fluorescein moiety, a cyanine moiety, an indocyanine green moiety, a triarylmethane, or a coumarin moiety. In embodiments, Dye3 is a rhodamine moiety, a sulforhodamine 101 moiety, a fluorescein moiety, a cyanine moiety, an indocyanine green moiety, a triarylmethane, or a coumarin moiety. In embodiments, Dye1 is a rhodamine moiety. In embodiments, Dye1 is a sulforhodamine 101 moiety. In embodiments, Dye1 is a fluorescein moiety. In embodiments, Dye1 is a cyanine moiety. In embodiments, Dye1 is an indocyanine green moiety. In embodiments, Dye1 is a triarylmethane. In embodiments, Dye1 is a coumarin moiety. In embodiments, Dye2 is a rhodamine moiety. In embodiments, Dye2 is a sulforhodamine 101 moiety. In embodiments, Dye2 is a fluorescein moiety. In embodiments, Dye2 is a cyanine moiety. In embodiments, Dye2 is an indocyanine green moiety. In embodiments, Dye2 is a triarylmethane. In embodiments, Dye2 is a coumarin moiety. In embodiments, Dye3 is a rhodamine moiety. In embodiments, Dye3 is a sulforhodamine 101 moiety. In embodiments, Dye3 is a fluorescein moiety. In embodiments, Dye3 is a cyanine moiety. In embodiments, Dye3 is an indocyanine green moiety. In embodiments, Dye3 is a triarylmethane. In embodiments, Dye3 is a coumarin moiety.

In embodiments, Q1 and Q2 each include the same quenching moiety. In embodiments, Q1 and Q2 each include different quenching moieties. In embodiments, the FRET pairs described herein include spectrally distinct FRET pairs (e.g., the FRET pair including Dye2 and Q1 includes a spectrally detectable label and/or quenching moiety compared with the FRET pair including Dye3 and Q2). Non-limiting examples of quenching moieties include diarylethenes (e.g., bisthienylethene derivatives), azines (e.g., azobenzenes, such as dabcyl derivatives)), photochromic quinones (e.g., phenoxynaphthacene quinone), spirooxazine, spirothiazines, mesoaldehyde 1-allyl-1-phenyl-2-phenylosazone, tetrachloro-1,2-ketonaphthalenone, thioindigoides, dinitrobenzylpyridine, and chromenes. In embodiments, the quenching moiety is a monovalent 4-(dimethylamino)azobenzene (DABCYL), monovalent dinitrophenyl, monovalent DABMI, monovalent malachite green, monovalent QSY™ 7 monovalent QSY™ 9, monovalent QSY™ 21, monovalent QSY 35 (“QSY™” quenchers available from Molecular Probes, Inc., Eugene, Oreg., see U.S. Pat. No. 6,329,205, incorporated herein by reference) and the black hole quenchers (BHQ) taught in WO2001/086001, incorporated herein by reference. In embodiments, the Q1 and Q2 are each independently

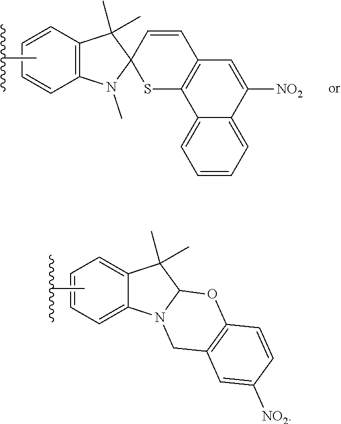

Additional quenching moieties capable of being covalently linked include 4-dimethylaminoazobenzene (DABCYL), Black Hole Quenchers (BHQ-1, BHQ-2), methyl red, proflavin, methyl blue, nitrobenzoxadiazole (NBD), tryptophan, anthraquinone derivatives, malachite green, porphyrins, and tetramethylrhodamine (TAMRA). Additional non-limiting examples of quenching moieties include monovalent species of Dabsyl (dimethylaminoazobenzenesulfonic acid), Black Hole Quenchers (BHQ) (e.g., (BHQ), BHQ-2, and BHQ-3), BMN Quenchers (e.g., BMN-Q460, BMN-Q535, BMN-Q590, BMN-Q620, BMN-Q650) Qxl, Tide Quenchers (e.g., TQ2, TQ3), Iowa black FQ, Iowa black RQ, Deep Dark Quencher (e.g., DDQ I, DDQ II), or IRDye QC-1. In embodiments, the detectable moiety is BMN-Q460, Dabcyl, DDQ-I, BMN-Q535, HHQ-1, TQ2, BMN-Q620, BMN-Q590, BHQ-2, TQ3, BMN-Q650, or BBQ-650. In embodiments, the detectable moiety is a quenching moiety capable of quenching fluorescence in the range of 400-530 nm, 480-580 nm, 550-650 nm, 480-720 nm, or 550-720 nm.

In embodiments, the kit further includes a cleaving agent. In embodiments, the cleaving agent is a reducing agent. In embodiments, the cleaving agent is an oxidizing agent. In embodiments, the cleaving agent is a phosphine containing agent. In embodiments, the cleaving agent is a thiol containing agent. In embodiments, the cleaving agent is di-mercaptopropane sulfonate (DMPS). In embodiments, the cleaving agent is aqueous sodium sulfide (Na₂S). In embodiments, the cleaving agent is Tris-(2-carboxyethyl)phosphines trisodium salt (TCEP), tris(hydroxypropyl)phosphine (THPP), guanidine, urea, cysteine, 2-mercaptoethylamine, or dithiothreitol (DTT). In embodiments, the cleaving agent is an acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the method includes contacting the compound (e.g., a compound described herein) with a reducing agent. In embodiments, the kit further includes a wash buffer and an assay buffer.

In embodiments, the kit further includes a sample collection device. In embodiments, the sample collection device includes EDTA or heparin (e.g., when the sample is obtained from plasma). In embodiments, following collection the sample is stored at less than −20° C. In embodiments, the sample collection device is a serum separator tube (SST). In embodiments, the sample collection device is a vial.

In some embodiments, the kit includes instructions for sample collection. In embodiments, the kit includes instructions and information on fasting, diet, and medication restrictions. In embodiments, the kit includes reagents (e.g., ethanol), sterilizing swabs, a marking pen, cotton, distilled water, spoons, scoops, tongue depressor, forceps, tongs, spatula, pipettes, Moore swabs (i.e., gauze strips), sponges, containers, and/or plastic bags. In embodiments, the kit includes an ice pack. In embodiments, the individual components of the kit can be alternatively contained either together in one storage container or separately in two or more storage containers (e.g., separate bottles or vials). In embodiments, the kit includes nucleotides in a buffer. In embodiments, the kit includes a buffer. For example, the sequencing solution and/or the chase solution may include a buffer such as ethanolamine (EA), tris(hydroxymethyl)aminomethane (Tris), glycine, a carbonate salt, a phosphate salt, a borate salt, 2-dimethyalaminomethanol (DMEA), 2-diethyalaminomethanol (DEEA), N,N,N′,N′-tetramethylethylenediamine (TEMED), and N,N,N′,N′-tetraethylethylenediamine (TEEDA), and combinations thereof. For example, the buffer may Tris-HCl (pH 9.2 at 25° C.), ammonium sulfate, MgCl₂, 0.1% Tween® 20, and dNTPs.

In embodiments, the collection device is a collection device described herein. For example, a collection device may include a suitable vessel, container, or material, such as a microfluidic paper-based analytical device (μPAD), cotton swab, transdermal patch, or device configured to collect and store a fluid or biological sample. In some embodiments, the collection device includes a nasal swab, nasopharyngeal swab, oropharyngeal swab, throat swab, buccal swab, oral fluid swab, stool swab, tonsil swab, vaginal swab, cervical swab, blood swab, or wound swab.

In embodiments, the kit is stored for 1 to 90 days. In embodiments, the kit is stored for greater than 90 days. In embodiments, the kit is stored for 1 to 30 days. In embodiments, the kit is stored for 1, 5, 7, 14, 21, 30, 45, 60, 75, 90, or more days. In embodiments, the kit is stored at less than about 25° C. In embodiments, the kit is stored at less than about 5° C. In embodiments, the kit is stored at about 4° C. In embodiments, the kit is stored in the dark (e.g., in the absence of light, such as visible light or UV light). In embodiments, the kit is stored at 2-8° C. In embodiments, the kit is stored for at least 1 day, at least 2 days, at least 3 days, or at least 7 days. In embodiments, the kit is stored for about 1 week, about 2 weeks, about 3 weeks, about 4 weeks, about 5 weeks, about 6 weeks, about 7 weeks, or about 8 weeks. In embodiments, the kit is stored for about 1 month, about 2 months, about 3 months, about 4 months, about 5 months, about 6 months, about 7 months, about 8 months, about 9 months, about 10 months, about 11 months, or about 12 months. In embodiments, the kit is stored at about 2° C.-8° C., about 20° C.-30° C., or about 4° C.-37° C. In embodiments, the kit is stored at about −5° C. to −30° C. and protected from light. In embodiments, the kit is stored at about 2° C.-8° C. and protected from light. In embodiments, the kit is stored at about 20° C.-30° C. and protected from light. In embodiments, the kit is stored at or about 4° C.-37° C. and protected from light.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In embodiments, the kit is designed for staining tissue samples for imaging and detecting target molecules (e.g., proteins) can be significantly expanded beyond the inclusion of fluorophores. For instance, the kit can include eosin and hematoxylin, which are classic histological stains. Eosin, a red dye, typically stains acidic components of the cell such as cytoplasmic proteins, while hematoxylin, a basic dye, binds to nucleic acids, coloring the cell nucleus blue. This combination is widely used in histopathology for detailed tissue structure visualization. Moreover, the kit can encompass stains such as acridine orange, a nucleic acid-selective fluorescent cationic dye, and Bismarck brown, which is often used for staining backgrounds in histological tissue sections. Carmine, another potential inclusion, is a natural red dye used for staining glycogen, while Coomassie blue is a popular choice for protein staining in gel electrophoresis. Crystal violet, a triarylmethane dye, can be included for staining cell walls and nuclei, and DAPI, a fluorescent stain that binds strongly to A-T rich regions in DNA, is useful in fluorescence microscopy. Ethidium bromide, a fluorescent intercalator, is also a valuable addition for its role in nucleic acid staining, especially in gel electrophoresis. Further, the kit can include acid fuchsine, used in Masson's trichrome stain; Hoechst stains, which are cell-permeable, DNA-specific blue fluorescent dyes; and iodine, commonly used in Gram staining and for staining starch in plant cells. Methyl green and methylene blue, both traditional histological stains, can be included for their affinity towards nucleic acids. Neutral red, a vital stain that accumulates in lysosomes, Nile blue and Nile red, both used for staining lipids, and osmium tetroxide, a heavy metal stain for lipid bilayers in electron microscopy, can be part of the kit. Propidium iodide, a popular red-fluorescent nuclear and chromosome counterstain, along with rhodamine, may be utilized. Safranin, commonly used in Gram staining, can be included for its ability to stain cell components like nuclei, cytoplasm, and cell walls in various colors, enhancing the contrast and detail in tissue imaging.

In embodiments, the kit includes a microplate described herein. In embodiments, the kit includes a flow cell, for example as described herein.

In an aspect is provided a device configured to detect multiple targets as described herein. In embodiments, the device includes a microscope. Examples of suitable microscopes include, but are not limited to, the Zeiss Axioscope 5 multichannel microscope (Carl Zeiss Microscopy, LLC, White Plains, N), the Olympus BX63 automated microscope (Olympus Scientific Solutions Americas Corp., Waltham, MA), and the Nikon Eclipse Ti2 microscope (Nikon Instruments, Inc., Melville, NY). In embodiments, the device includes one or more light sources, one or more objective lenses, one or more sample carriers (e.g., sample holders, sample stages, and/or translation stages), one or more tube lenses, one or more image sensors or cameras, one or more processors or controllers, one or more additional optical components (e.g., lenses, mirrors, prisms, beam-splitters, optical filters, colored glass filters, narrowband interference filters, broadband interference filters, dichroic reflectors, diffraction gratings, apertures, shutters, optical fibers, optical waveguides, and/or acousto-optic modulators), or any combination thereof. In embodiments, the device includes a focus mechanism, e.g., an autofocus mechanism. In embodiments, the device is be configured to perform multichannel imaging, e.g., multichannel fluorescence imaging including the use of excitation light at one or more excitation wavelengths, and imaging the emitted fluorescence at two or more different emission wavelengths.

In embodiments, the device includes one or more image sensors (or cameras) that may be the same or may be different, and may include any of a variety of image sensors including but not limited to, photodiode arrays, charge-coupled device (CCD) sensors or cameras, or complementary metal-oxide-semiconductor (CMOS) image sensors or cameras. In embodiments, the one or more image sensors may comprise one-dimensional (linear) or two-dimensional pixel array sensors. In embodiments, the one or more image sensors may comprise monochrome image sensors (e.g., configured to capture greyscale images) or color image sensors (e.g., configured to capture RGB or color images). In embodiments, the one or more image sensors may be used to capture single images, e.g., a single image for each cycle of a plurality of cycles. In embodiments, the one or more image sensors may be used to capture a series of images, e.g., a series of images during each cycle of a plurality of cycles. In embodiments, a series of images may include images (or video frames) that correspond to images captured before, during, and/or after an event, e.g., before, during, and/or after addition of a probe to the sample being imaged. In embodiments, a series of images includes 2 images, 3 images, 4 images, 5 images, 10 images, 20 images, 30 images, 40 images, 50 images, 100 images, 200 images, 300 images, 400 images, 500 images, 1,000 images, or more than 1,000 images.

In embodiments, the device is configured to perform volumetric imaging (or optical sectioning). In embodiments, the imaging includes the acquisition of a plurality (or “stack”) of two-dimensional (2D) images to form a three-dimensional (3D) representation of the sample, where each two-dimensional image is aligned with the other images of the plurality in the sample plane (e.g., the X-Y plane), but is offset from the other two-dimensional images in a direction parallel to the optical axis of the imaging module (e.g., in the Z-direction). In embodiments, the stack of images may be acquired sequentially. In embodiments, the stack of images may be acquired simultaneously.

In embodiments, the device is configured to control the delivery of fluids such as reagents and/or buffers to a sample, e.g., a sample contained within the flow cell. In embodiments, the one or more fluidics controllers may be configured to control volumetric flow rates for one or more fluids or reagents, linear flow velocities for one or more fluids or reagents, mixing ratios for one or more fluids or reagents, or any combination thereof. Fluidics modules may include one or more fluid flow sensors (e.g., flow rate sensors, pressure sensors, etc.), one or more fluid flow actuators (e.g., pumps), one or more fluid flow control devices (e.g., valves), one or more processors (and associated electronics), tubing and connectors to connect the one or more fluidics modules to one or more flow cells, or any combination thereof.

### EXAMPLES

### Example 1. Multiplex Detection

Early disease diagnosis plays an important role in effective treatment. Clinical evidence based on a single analyte or single biomarker is typically not adequate for a confident diagnosis of a disease or monitoring treatment. While commercial single-plex techniques such as enzyme-linked immunosorbent assay (ELISA) (e.g., sandwich immunoassays) and biomarker kits can accurately detect a single analyte, the monitoring of more complex, multifactorial diseases such as cancer, autoimmune, and neurodegenerative diseases require the analysis of multiple biomarkers in order to confidently address the underlying disease (e.g., deciding best treatment options, tracking disease progression and response to therapy, and formulating prognoses). Multiplex detection methods, such as those described herein, confer several advantages over widely used single-plex assays including increased efficiency, greater output per sample volume, and higher throughput. For almost 50 years, immunoassays have allowed for sensitive and specific detection of analytes of interest in biological samples. Typical immunoassays include staining biomolecules, which include binding an antibody (Ab) to a target analyte and detecting the Ab directly (e.g., the Ab is labeled with a fluorophore, referred to as a primary antibody) or indirectly (i.e., a second labeled antibody binds to the first antibody and is detected). Staining is limited by spectral overlap.

Spectral overlap occurs when two or more fluorophores used to detect different targets emit similar wavelengths of light that overlap in the detection spectrum. The resulting light intensity signals for the different targets become muddled and make it difficult or impossible to distinguish between them. To minimize spectral overlap, different fluorophores with contrasting colors should be used for each target so that the individual spectral signals can be clearly distinguished. Generally, acceptable thresholds of spectral overlap for detection are set such that the emission peaks of the different fluorophores minimally overlap, e.g., less than 25% overlap. Because emission spectra have a variety of shapes, widths, and degrees of overlap, the degree of spectral overlap varies greatly. In order to maximize separation between fluorophores for multiplexing, it is important to choose dyes with distinct emission spectra and wavelengths that do not overlap with one another. Given the narrow range (e.g., typical emission wavelengths range from 400-700 nm) there are a finite number of fluorophores one can use. For example, detecting five targets requires five different fluorophores: (i) Alexa Fluor® 488 (ex/em wavelength of 495/520 nm); (ii) Cy®3 (ex/em wavelength of 559/573 nm), (iii) Cy®5 (ex/em wavelength of 649/670 nm); (iv) Cy®7 (ex/em wavelength of 759/780 nm), and (v) DyLight™ 649 (ex/em wavelength of 646/671 nm). Practically, this limits approaches to detect four to five biomolecules simultaneously.

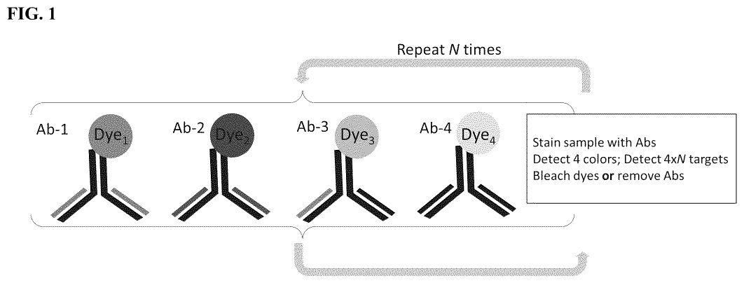

Strategies to overcome this limitation enabling multi-biomolecule detection include cyclical staining protocols, wherein after each round of staining the fluorophores are inactivated or the bound antibodies are removed. For example, see FIG. 1 for an example of the set of detection antibodies and the cyclic workflow. Removing antibodies include incubating the sample with an enzyme (e.g., a protease) and enabling enzymatic digestion of the antibodies. However, antibody digestion is a harsh procedure. For example, enzymatic digestion typically degrades antigens and damages cell structures within the sample, limiting the number of cycles one may perform. Additionally, antibodies with different isotypes are digested at different rates, resulting in inconsistent removal or lengthy digestion times.

Alternatively, between each cycle, the fluorophore may be inactivated or destroyed, which involves contacting the sample with a broad range of solutions, chemical agents, and/or modulating the temperature. For example, some protocols include using a boiling antigen retrieval solution (T6th et. al J Histochem Cytochem. 2007; 55:545-54), basic hydrogen peroxide (Lin et al. Curr Protoc Chem Biol. 2016 Dec. 7; 8(4):251-264), or a mixture of strong reducing agents and detergents (Gendusa et al. J Histochem Cytochem. 2014; 62:519-31). Alternatively, after detecting the primary antibodies in the tissue, the fluorophore is inactivated by contacting the tissue with ultraviolet (UV) light, alkaline solutions, or sodium borohydride (NaBH₄). The bleaching reagents also negatively impact the integrity of the sample, resulting in cell loss and destruction.

The cyclic incubation with bleaching reagents and antibodies overcomes the limitation of spectral overlap of fluorophores but prolongs the imaging process proportionally to the number of markers included. For example, one cycle (including overnight antibody incubation plus imaging and inactivation time) typically requires 24 hours (Lin et al. Curr Protoc Chem Biol. 2016 Dec. 7; 8(4):251-264). Limited to four targets per cycle, imaging 50 targets in a single sample would take about 12 days. Indeed, Jia-Ren Lin and colleagues validated this when detecting 60 different targets in a sample, it required greater than 14 days of preparation, imaging, and analysis (Jia-Ren Lin et al. eLife 7:e31657). Detecting multiple targets in multiple different samples has thus far remained an impossible task.

Simultaneous measurement of multiple analytes from a single sample results in a significant cost, time, and sample savings. Herein, we describe serially revealing labeled probes for detecting multiple biomolecules in a sample, resulting in significant time savings while maintaining sample integrity. The method described herein utilize probe sets containing orthogonal cleaving sites, thereby overcoming the spectral limitations by temporally controlling probe detection. A set of probes includes at least (i) a first probe linked to a dye via a cleavable linker, having the formula [probe-1]-X₁-dye; and (ii) a second probe linked to a dye via a second cleavable linker and a quenching moiety, having the formula [probe-2]-X₂-dye-X₁-quencher, wherein X₁ and X₂ are different cleavable sites. The set may be expanded to include additional probes, having the generic formula: [probe-N]-X_{(N+1)}-dye-X_{(N)}-quencher, wherein X_{(N+1)} is different cleavable site relative to X_{N}. The probe set may include a final probe that includes only a quencher, having the formula [probe-N]-dye-X_{(N)}-quencher. Careful design of the probes ensures the cleavage within each probe set is sequential.

FIGS. 2A-2B illustrate some of the contemplated probes for use with the methods described herein. Illustrated in FIG. 2A is a set of four detection antibodies (Ab-1, Ab-2, Ab-3, and Ab-4) with serially cleaving quenchers. The first detection antibody includes a dye and a cleavable linker, X₁. The second antibody includes a second cleavable linker X₂, the dye, the first cleavable linker X₁, and a quencher. The third antibody includes a third cleavable linker X₃, the dye, the second cleavable linker X₂, and a quencher. The fourth antibody includes a fourth cleavable linker X₂, the dye, the third cleavable linker X₃, and a quencher.

FIG. 2B provides analogous oligonucleotide probes (OPs) to the specific binding reagent (e.g., antibody) probes of FIG. 2A. The first oligonucleotide probe includes a dye and a cleavable linker, X₁. The second oligonucleotide probe includes a second cleavable linker X₂, the dye, the first cleavable linker X₁, and a quencher. The third oligonucleotide probe includes a third cleavable linker X₃, the dye, the second cleavable linker X₂, and a quencher. The fourth oligonucleotide probe includes a fourth cleavable linker X₂, the dye, the third cleavable linker X₃, and a quencher.

An example workflow for detecting multiple targets in a sample using the serial reveal method is provided in FIG. 3. The set of probes are all applied to the sample (e.g., a cell) and allowed to bind to their respective targets (e.g., proteins, such as antibodies or cytokines), followed by and the serially revealing the dyes by cleaving off their associated quenchers. For example, in the first cycle, the first dye is detected (i.e., Dye 1 of Ab-1), followed by cleaving the first cleavable linker. The first dye is removed from Ab-1, and the quencher moiety is removed from Ab-2, as a result of cleaving the first cleavable linker (e.g., depicted as X₁ in FIGS. 2A and 2B) and thus enabling detection of Dye 2 from Ab-2. Next, the second cleavable linker is cleaved (e.g., depicted as X₂ in FIGS. 2A and 2B), removing the dye (i.e., Dye 2) from Ab-2 and exposing Dye 3 from Ab-3. Next, the third cleavable linker is cleaved, removing the dye (i.e., Dye 3) from Ab-3 and enabling detection of Dye 4 from Ab-4.

A significant advantage is realized when expanding the set of probes to include additional spectrally distinct fluorophores. For example, a first set of four probes targeting four different targets (Target-1, Target-2, Target-3, and Target-4) includes a first dye (e.g., red) and a second set of four probes targeting four different targets (Target-5, Target-6, Target-7, Target-8) include a second dye (e.g., blue). The sample is incubated with both sets of probes and allowed to bind to their respective targets. During the first cycle, both the red and the blue dyes are detected, enabling detection of Target-1 and Target-5, after which the first cleavable site, X₁, is cleaved. The next cycle detects the red and blue dyes which are associated with Target-2 and Target-6, respectively. The process may be repeated to detect all 8 probes; see Table 1.

TABLE 1
Representation of the data collected for two probes sets, wherein
each probe set includes four probes and a different fluorophore
(i.e., the first set of 4 probes includes a red fluorophore
and the second set of 4 probes include a blue fluorophore).
A “1” in the table indicates the fluorescent emission
is detected, whereas a “0” means no emissions are detected.

| | Cycle 1: | Cycle 2: | Cycle 3: | |
|---|---|---|---|---|
| | Detect then | Detect then | Detect then | Cycle 4: |
| | cleave X₁ | cleave X₂ | cleave X₃ | Detect |
| Probe set 1 | Target-1 | Target-2 | Target-3 | Target-4 |
| Red | 1 | 0 | 0 | 0 |
| | 0 | 1 | 0 | 0 |
| | 0 | 0 | 1 | 0 |
| | 0 | 0 | 0 | 1 |
| Probe Set 2 | Target-5 | Target-6 | Target-7 | Target-8 |
| Blue | 1 | 0 | 0 | 0 |
| | 0 | 1 | 0 | 0 |
| | 0 | 0 | 1 | 0 |
| | 0 | 0 | 0 | 1 |

Greater multiplexing is realized if including additional colors. For example, expanding the first two sets described supra, (i.e., a first set of four probes targeting four different targets (Target-1, Target-2, Target-3, and Target-4) includes a first dye (e.g., red) and a second set of four probes targeting four different targets (Target-5, Target-6, Target-7, Target-8) include a second dye (e.g., blue)) may further include two additional sets of probes. A third set of four probes targeting four different targets (Target-9, Target-10, Target-11, and Target-12) includes a third spectrally distinct dye (e.g., yellow) and a fourth set of four probes targeting four different targets (Target-13, Target-14, Target-15, Target-16) include a fourth dye (e.g., green). A representation of the data collected for four probe sets enabling detecting 16 targets with a single incubation event as described above may be found in Table 2.

TABLE 2
Representation of the data collected for four probes sets, wherein
each probe set includes four probes and a different fluorophore.
A “1” in the table indicates the fluorescent emission
is detected, whereas a “0” means no emissions are detected.

| | Cycle 1: | Cycle 2: | Cycle 3: | |
|---|---|---|---|---|
| | Detect then | Detect then | Detect then | Cycle 4: |
| | cleave X₁ | cleave X₂ | cleave X₃ | Detect |
| Probe set 1 | Target-1 | Target-2 | Target-3 | Target-4 |
| Red | 1 | 0 | 0 | 0 |
| | 0 | 1 | 0 | 0 |
| | 0 | 0 | 1 | 0 |
| | 0 | 0 | 0 | 1 |
| Probe Set 2 | Target-5 | Target-6 | Target-7 | Target-8 |
| Blue | 1 | 0 | 0 | 0 |
| | 0 | 1 | 0 | 0 |
| | 0 | 0 | 1 | 0 |
| | 0 | 0 | 0 | 1 |
| Probe set 3 | Target-9 | Target-10 | Target-11 | Target-12 |
| Yellow | 1 | 0 | 0 | 0 |
| | 0 | 1 | 0 | 0 |
| | 0 | 0 | 1 | 0 |
| | 0 | 0 | 0 | 1 |
| Probe set 4 | Target-13 | Target-14 | Target-15 | Target-16 |
| Green | 1 | 0 | 0 | 0 |
| | 0 | 1 | 0 | 0 |
| | 0 | 0 | 1 | 0 |
| | 0 | 0 | 0 | 1 |

As described herein, cyclic immunofluorescent methods inactivate or destroy the fluorophore by contacting the sample with a broad range of solutions, chemical reagents, and/or modulating the temperature. Repeated contact with the solutions and reagents negatively impacts the integrity of the sample, resulting in cell loss and destruction. Utilizing known methods that capable of detecting five targets per round of staining requires 12 rounds of staining and 11 rounds of bleaching (i.e., detect 5 targets, bleach, detect 5 targets, bleach, etc.) to achieve multiplex detection of 60 targets. Utilizing the methods described herein 60 targets may be detected with 4 rounds of staining and 0 rounds of bleaching (e.g., utilizing the four sets of probes as described in Table 2, followed by bleaching after cycle 4). An example of multiple staining protocols is described below.

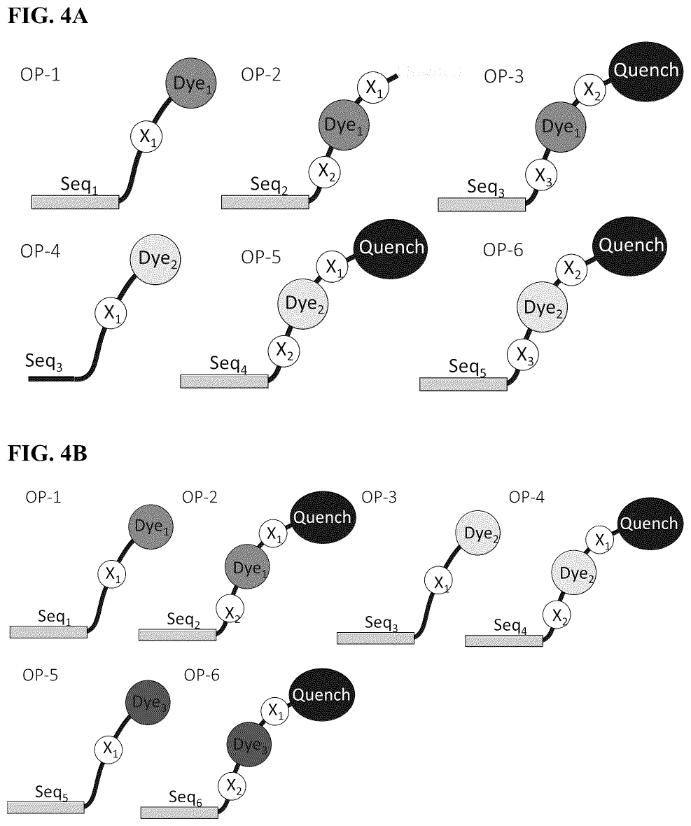

Alternative combinations are also contemplated herein. For example, FIGS. 4A-4B provide an example of some of the potential combinatorial sets of probes. FIG. 4A provides two set of probes. The first set of oligonucleotide probes include a first dye (e.g., green) and three orthogonal cleavable moieties. The second set of oligonucleotide probes include a second dye (e.g., yellow) and the three orthogonal cleavable moieties. Table 3 provides a representation of the data collected for a first round of staining using the probe set provided in FIG. 4A. Briefly, a first round of staining includes incubating both sets of probes with the sample under suitable staining conditions. The first target and the fourth target are detected in the first cycle, followed by cleaving the first cleavable linker. Next the second target and the fifth target are detected, followed by cleaving the second linker. Finally, the third target and the sixth target are detected in the third round. A second round of staining occurs, wherein the same dye colors and cleavable types (i.e., two colors, three cleavable linker types) but the probes include binding regions for target different targets, enabling six additional targets to be detected in the second round.

TABLE 3
Two colors-three orthogonal cleavable linker types for two
rounds of staining. A “1” in the table indicates
the fluorescent emission is detected, whereas a “0” means
no emissions are detected. Note, for the final cycle of the final
staining round, X₃ may not be cleaved.

| | | |
|---|---|---|
| Cycle 1: | Cycle 2: | Cycle 3: |
| Detect then | Detect then | Detect then |
| cleave X₁ | cleave X₂ | cleave X₃ |

First Staining Round

| Probe set 1 | Target-1 | Target-2 | Target-3 |
|---|---|---|---|
| Green | 1 | 0 | 0 |
| | 0 | 1 | 0 |
| | 0 | 0 | 1 |
| Probe Set 2 | Target-4 | Target-5 | Target-6 |
| Yellow | 1 | 0 | 0 |
| | 0 | 1 | 0 |
| | 0 | 0 | 1 |

Second Staining Round

| Probe set 3 | Target-7 | Target-8 | Target-9 |
|---|---|---|---|
| Yellow | 1 | 0 | 0 |
| | 0 | 1 | 0 |
| | 0 | 0 | 1 |
| Probe set 4 | Target-10 | Target-11 | Target-12 |
| Green | 1 | 0 | 0 |
| | 0 | 1 | 0 |
| | 0 | 0 | 1 |

Alternatively, three color-two cleavable linker, as illustrated in FIG. 4B provides additional means for detecting six targets per round of staining. For example, the first set includes a first dye (e.g., green) and two orthogonal cleavable moieties, X₁ and X₂. The second set includes a second dye (e.g., yellow) and the two orthogonal cleavable moieties, X₁ and X₂. Finally, the third set includes a third dye (e.g., blue) and the two orthogonal cleavable moieties, X₁ and X₂. Table 4 provides a representation of the data collected for a three color-two cleavable linker probe set.

TABLE 4
Data for a three color (green, yellow, blue) probe set
with two orthogonal cleavable linking types. A “1”
in the table indicates the fluorescent emission is detected,
whereas a “0” means no emissions are detected.

| | Cycle 1: | |
|---|---|---|
| | Detect then cleave | Cycle 2: |
| | X₁ | Detect |
| Probe set 1 | Target-1 | Target-2 |
| Green | 1 | 0 |
| | 0 | 1 |
| Probe Set 2 | Target-3 | Target-4 |
| Yellow | 1 | 0 |
| | 0 | 1 |
| Probe set 3 | Target-5 | Target-6 |
| Blue | 1 | 0 |
| | 0 | 1 |

Alternative configuration of probes are contemplated herein. For example, as illustrated in FIGS. 5A-5B, embodiments of molecular beacon antibody (MB-Ab) probes are provided. FIG. 5A provides a generic formula for constructing a set of N MB-Ab probes, where N signifies the quantity of MB-Ab probes. FIG. 5B probes an example set of MB-Ab probes, wherein the first antibody includes a first cleavable moiety denoted as X₁ (e.g., a first restriction enzyme motif). The second antibody includes the first cleavable moiety X₁ such that upon cleavage the quenching moiety (illustrated as an octagon) is removed to expose the dye (illustrated as a star). The second antibody also includes a second cleavable site, denoted as X₂ (e.g., a second restriction enzyme motif) that may be cleaved to remove the dye from the second antibody and remove the quenching moiety from the third antibody.

### Example 2. Detection of RNA Targets Outside of a Cell

Gaining a clearer understanding of the origin, state and fate of a cell in a physiological and immunological context is a pressing need within biological and biomedical research. RNA molecules are traditionally confined to the cytosolic and nuclear spaces of a cell, where it plays critical and conserved roles across nearly all biochemical processes. However recent understanding informs us that RNA is known to be exported from cells as extracellular RNA (exRNA) and to play a role in cell-to-cell communication. Monitoring cellular communications encoded by exRNA could reveal early signs of diseases such as cancer.

The methods described in Example 1 may be applied to a cell or tissue sample by serially revealing labeled oligonucleotide probes for detecting multiple RNA molecules on the surface of a cell, or otherwise outside of the cell within a sample. The method described herein utilize oligonucleotide probe sets containing orthogonal cleaving sites, thereby overcoming the spectral limitations. A set of oligonucleotide probes includes at least (i) a first probe linked to a dye via a cleavable linker, having the formula [oligo probe-1]-X₁-dye; and (ii) a second probe linked to a dye via a second cleavable linker and a quenching moiety, having the formula [oligo probe-2]-X₂-dye-X₁-quencher, wherein X₁ and X₂ are different cleavable sites. The set may be expanded to include additional probes, having the generic formula: [oligo probe-N]-X_{(N+1)}-dye-X_{(N)}-quencher, wherein X_{(N+1)} is different cleavable site relative to X_{N}. The probe set may include a final probe that includes only a quencher, having the formula [oligo probe-N]-dye-X_{(N)}-quencher. Careful design of the oligonucleotide probes ensures the cleavage within each probe set is sequential.

### Example 3. Serial Revealing Cell Paints

The detection and analysis of multiple biomolecules within the same cell or tissue section is crucial for understanding the phenotypic and functional architecture of healthy and diseased states. Traditional single-plex techniques, such as enzyme-linked immunosorbent assays (ELISA), are limited in their ability to provide comprehensive insights due to their focus on single analytes. In contrast, multiplexed detection methods offer the potential to simultaneously analyze multiple biomarkers, thereby providing a more holistic view of cellular and tissue states. However, existing multiplexed antibody-based techniques, including those employing fluorophores, metal markers, and DNA barcodes, face significant challenges. These challenges include the need for meticulous antibody validation, issues with spectral overlap, and the complex and time-consuming nature of sequential staining and bleaching protocols.

Fluorescent multiplexing techniques, such as multiplex immunofluorescence and tissue-based circular immunofluorescence, rely on labeling biomolecules with distinct fluorophores. While these methods can provide sensitive and specific detection, they are constrained by the limited spectral range available for fluorescence detection. Spectral overlap occurs when fluorophores emit light at similar wavelengths, making it difficult to distinguish between different targets. This overlap necessitates the use of dyes with minimal emission overlap, often restricting the number of biomarkers that can be simultaneously detected to four or five. Additionally, methods for removing or inactivating fluorophores after each round of staining, such as enzymatic digestion or chemical bleaching, can damage the sample and prolong the imaging process. As a result, detecting a large number of targets can become impractically lengthy and complex.

To address these limitations, advanced techniques like DNA barcoding have been developed, enabling higher multiplexing capabilities by avoiding spectral limitations. However, these methods typically require complex probe design and hybridization protocols, which can be cumbersome and expensive. Additionally, despite these advances, the sole reliance on antibodies remains a hurdle, as it requires rigorous validation to ensure accuracy and reproducibility. Consequently, there is a pressing need for innovative approaches that can overcome these challenges, enabling efficient and accurate multiplex detection of biomolecules in situ, without the drawbacks associated with current technologies.

The present disclosure addresses the limitations of existing multiplexed biomolecule detection methods by introducing an advanced cell painting technique, capable of being used existing spatial biology platforms (e.g., the G4X™ Platform or ImageXpress® Confocal HT.ai system). This innovative approach combines the principles of traditional cell painting with the enhanced capabilities of cleavable linkers and sequential staining cycles. By integrating these elements, the invention enables the detection of a significantly larger number of cellular structures and biomarkers within the same sample, overcoming the spectral overlap and optical cross-talk issues inherent in conventional fluorescence-based methods.

Described herein is the use of cleavable linkers that connect targeting molecules, such as phalloidin or wheat germ agglutinin (WGA), to fluorescent dyes. These linkers can be cleaved through specific chemical, enzymatic, or photolytic reactions, allowing for the sequential removal of dyes after imaging. This cyclical process of staining, imaging, and cleaving is akin to painting a portrait or screen printing, where one color is added at a time to build a complete image. Similarly, each round of staining adds new layers of information about the cellular structures, ultimately revealing a comprehensive and detailed picture of the cell or tissue with all structures resolved. By employing four distinct fluorescent dyes in each cycle and minimizing optical cross-talk by using only two dyes per cycle when necessary, the system can achieve high-resolution, high-throughput imaging of numerous cellular components and biomarkers.

The mushroom toxin phalloidin is a small bicyclic peptide consisting of seven amino acids with a molecular weight of 789. Phalloidin binds to both large and small filamentous actin (F-actin) with high affinity, and compared to actin-specific antibodies, the non-specific binding of phalloidin is negligible, thus providing minimal background and high contrast during cellular imaging. Phalloidin-dye conjugates have been described previously, for example Capani et al Journal of Histochemistry & Cytochemistry. 2001; 49(11):1351-1361, and including a cleavable site in the linker to the fluorophore enables the conjugate to be used in the method described herein. For example, the probe may have the structure:

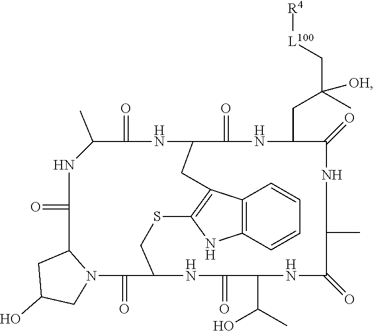

where L¹⁰⁰ is the cleavable linker and R⁴ is a fluorophore moiety.

The method also incorporates automated imaging and image analysis software, enhancing the efficiency and reproducibility of the staining and imaging process. This automation reduces manual intervention, minimizes potential errors, and facilitates large-scale studies. The resulting high-dimensional data can be integrated and analyzed to provide comprehensive profiles of cellular phenotypes, enabling detailed studies of cellular behavior, disease mechanisms, and treatment responses.

The phenotypic profile of a cell reveals the biological state of a cell. More specifically, the phenotypic profile can be used to interrogate biological perturbations because the cellular morphology is influenced by factors such as metabolism, genetic and epigenetic state of the cell, and environmental cues. In addition, it can be used to characterize healthy cells from diseased cells. Because a phenotypic profile is an aggregation of a large number of measurements, it is sensitive to deviations or changes to those features extracted using cellular paints. To create a profile of the cells, all of the features from the different organelles that are imaged and analyzed using commercially available cell imaging software (e.g., CellProfiler™) In morphological profiling, measured features include staining intensities, textural patterns, size, and shape of the labeled cellular structures, as well as correlations between stains across channels, and adjacency relationships between cells and among intracellular structures.

Existing cell paints, described in Table 5, are employed to target specific biomolecules. In current cell painting approaches, fluorescent dyes are conjugated to targeting molecules through covalent bonding, ensuring specific and stable labeling of cellular structures. The attachment process typically involves the use of chemical linkers that form a stable covalent bond between the dye and the targeting molecule. For example, phalloidin, which binds specifically to actin filaments, is covalently linked to a fluorescent dye like Alexa Fluor® 488 using a reactive group on the dye that reacts with a functional group on phalloidin. Similarly, wheat germ agglutinin (WGA), which targets the plasma membrane, is conjugated to a fluorescent dye through a linker that attaches to its glycoprotein-binding sites. This covalent linkage ensures that the dye remains firmly attached to the targeting molecule during the staining, imaging, and any subsequent washing steps, providing consistent and reliable fluorescence labeling of the intended cellular structure.

TABLE 5
Commercially available cell paints

| Targeting | | Cell Structure |
|---|---|---|
| Molecule | Fluorescent Dye | Targeted |
| Phalloidin | Various (e.g., Alexa Fluor ® | Actin filaments |
| | 488, Alexa Fluor ® 568) | |
| Wheat Germ | Various (e.g., Alexa Fluor ® | Plasma membrane |
| Agglutinin | 488, Alexa Fluor ® 594) | |
| (WGA) | | |
| MitoTracker ® | Various (e.g., MitoTracker ® | Mitochondria |
| | Red CMXRos, MitoTracker ® | |
| | Green FM) | |
| ER-Tracker ™ | Various (e.g., ER-Tracker ™ | Endoplasmic |
| | Red, ER-Tracker ™ Green) | reticulum |
| Concanavalin | Various (e.g., Alexa Fluor ® | Endoplasmic |
| A | 350) | reticulum |
| Golgi-Tracker ™ | Various (e.g., BODIPY ® FL | Golgi apparatus |
| | C5-Ceramide) | |
| LysoTracker ® | Various (e.g., LysoTracker ® | Lysosomes |
| | Green DND-26, LysoTracker ® | |
| | Red DND-99) | |
| | CytoFix ™ Red | |
| Annexin V | Various (e.g., Annexin V Alexa | Phosphatidylserine |
| | Fluor ® 488, Annexin V FITC) | (apoptosis marker) |
| Concanavalin | Various (e.g., Alexa Fluor ® | Cell surface |
| A (ConA) | 488, Alexa Fluor ® 594) | carbohydrates |
| Transferrin | Various (e.g., Alexa Fluor ® | Transferrin |
| | 488, Alexa Fluor ® 568) | receptors |
| Lectins (e.g., | Various (e.g., Alexa Fluor ® | Specific carbohy- |
| PNA, UEA-1) | 488, Alexa Fluor ® 594) | drate structures |

The method may be useful in detecting biomolecules such as proteins and nucleic acid molecules, organelle structures such as the Golgi Apparatus, and also the cytoskeleton. The cytoskeleton is a network of different protein fibers (e.g., actin and myosin) that maintains the shape and position of the organelles within a cell. The cytoplasm, a fluid which can be rather gel-like, surrounds the nucleus, is considered an organelle.

Additional organelles detectable using the methods and compositions described herein include the Endoplasmic Reticulum (ER), which is a network of membranes that forms channels that criss-crosses the cytoplasm utilizing its tubular and vesicular structures to manufacture various molecules. The ER includes small granular structures called ribosomes useful for the synthesis of proteins. Smooth ER makes fat compounds and deactivates certain chemicals like alcohol or detected undesirable chemicals such as pesticides. Rough ER makes and modifies proteins and stores them until notified by the cell communication system to send them to organelles that require the substances. Typically, all healthy cells in humans, except erythrocytes (red blood cells) and spermatozoa, are equipped with endoplasmic reticulum. The Golgi apparatus (also referred to as a Golgi complex) consists of one or more Golgi bodies which are located close to the nucleus and consist of flattened membranes stacked atop one another like a stack of coins. The Golgi apparatus prepares proteins and lipid (fat) molecules for use in other places inside and outside the cell. Lysosomes are membrane-enclosed organelles that have an acidic interior (pH ˜4.8) and can vary in size from 0.1 to 1.2 μm. Lysosomes house various hydrolytic enzymes responsible for digesting biopolymers such as proteins, peptides, nucleic acids, carbohydrates and lipids. Ribosomes are tiny spherical organelles distributed around the cell in large numbers to synthesize cell proteins. They also create amino acid chains for protein manufacture. Ribosomes are created within the nucleus at the level of the nucleolus and then released into the cytoplasm.

The methods and compositions described herein revolutionizes cell painting techniques by introducing the use of cleavable linkers between targeting molecules and fluorescent dyes. Prior to this disclosure, the use of cleavable linkers was avoided due to concerns over stability issues, as the linkers needed to be robust enough to withstand the staining and imaging processes yet easily cleavable when desired. The invention overcomes these stability challenges by utilizing designed cleavable linkers that maintain the stability of the dye-targeting molecule complex during imaging and can be selectively cleaved using specific chemical, enzymatic, or photolytic reactions. This innovative approach enables multiple rounds of staining and imaging, significantly expanding the multiplexing capacity and allowing for the detection of a greater number of cellular structures and biomarkers within the same cell or tissue sample.

### Example 4. Imaging a Multiplex Tonsil Tissue Sample

To image and analyze a multiplex tonsil tissue sample using a combination of intrinsic (e.g., Hoescht 33342) and non-intrinsic ([targeting molecule]-[cleavable linker (CL)]-[fluorophore]) cell paints, employing cleavable linkers for sequential staining and imaging cycles. By spatially separating the dyes, we minimize optical cross-talk and maximize detection clarity. To begin, the fixed and prepared tonsil tissue sample is subjected to an initial round of staining using a set of cell paints and immunostains designed to target specific cellular components. The first set includes:
Endoplasmic Reticulum: Concanavalin A (ConA)-CL-Alexa Fluor® 532 (emission: 532 nm)
Golgi Apparatus: Wheat germ agglutinin (WGA)-CL-Alexa Fluor® 594 (emission: 594 nm)
F-Actin: Phalloidin-CL-Alexa Fluor® 647 (emission: 647 nm)
Lysosomes: LysoTracker-CL-Alexa Fluor® 680 (emission: 680 nm)

Once the tissue is stained, it is imaged to capture the fluorescence signals from each dye. Following the initial imaging, the tissue sample undergoes treatment with specific cleavage reagents designed to remove the fluorescent dyes linked through cleavable linkers. The sample is then thoroughly washed to ensure complete removal of the cleaved dyes, preparing it for the next cycle of staining. In the second cycle, the tissue is stained with a new set of cell paints targeting additional structures, each conjugated with non-overlapping dyes to avoid optical cross-talk. This second set includes:
Nucleus: Hoechst 33342 (intrinsic, excitation/emission: 387/447 nm)
Nucleoli: SYTO 14 green fluorescent nucleic acid stain (intrinsic, emission: 531/593 nm)
Mitochondria: MitoTracker Deep Red (intrinsic, emission: 628/692 nm)
Transferrin Receptors: Transferrin-CL-Alexa Fluor 532 (emission: 532 nm)
Nuclear Envelope: Anti-Lamin A/C-CL-Alexa Fluor 594 (emission: 594 nm)
Cell Surface Receptors: Anti-CD3-CL-Alexa Fluor 422 (emission: 422 nm)

The tissue is then imaged again. After imaging, the dyes are cleaved, and the tissue is prepared for additional cycles, or detection modes, if necessary. This process of staining, imaging, and cleavage is repeated for subsequent cycles, each time introducing new cell paints to target different cellular components as illustrated in FIG. 6. Note, intrinsic stains such as Hoechst 33342 and SYTO 14, should be included in the final set so as not to interfere with detection in intervening staining cycles.

Each cycle ensures that only non-overlapping dyes are used to maintain clear separation of signals. For example, following one or more cycles using the cleavable conjugates described supra one can use traditional (i.e., non-cleavable) staining agents, such as primary antibodies (e.g., beta tubulin monoclonal antibody (ThermoFisher Scientific, 32-2600), anti-clathrin heavy chain antibody (abeam, ab21679), and anti-caveolin-1 antibody (abeam, ab2910) coupled with secondary antibody-oligonucleotide conjugates. For example, protocols for traditional immunostaining may be found in Civitci, F. et al. Protoc. Exch. doi.org/10.21203/rs.3.pex-1069/v1 (2020).

After all cycles are completed, the imaging data from each cycle are integrated using commercially available image analysis software. This software aligns the images from different cycles to create a comprehensive map of the cellular structures and biomarkers within the tonsil tissue. The data are then analyzed to quantify the expression and spatial distribution of the targeted components. By sequentially applying cell paints and utilizing cleavable linkers, this method allows for the imaging of a tonsil tissue sample, providing detailed and comprehensive visualization of various cellular components without the limitations of spectral overlap. The high-content imaging system captures high-resolution images, and the integrated data analysis offers insights into the cellular architecture and biomarker distribution within the tissue, facilitating a deeper understanding of tonsil tissue structure and function.

To facilitate the visualization of organelle and related target data commercially available software (e.g., TissueMaker®, TissueFAXS™, THUNDER™) can allow users to dynamically generate a visual interpretation of data. For example, a typical software may present a user interface with a three-dimensional representation of the cell and/or tissue. For example, the method may further include stitching. Stitching combines multiple field of view (FOV) into a single image. Stitching can be performed using a variety of techniques. For example, one approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and target biomolecule coordinates can be transferred to the combined image based on the horizontal and vertical shift. For the reconstruction of 3D tissues, several computational methods such as PASTE, PASTE2, SLAT, and SPACEL can be utilized. These methods and algorithms typically involve aligning detected targets between different slices and performing coordinate transformation and rotation of different slices to achieve a 3D structure composed of multiple slices.

### Example 5: Profiling Genome Editing Efficiency

The evolution of gene editing towards clinical practice has developed through recent advancements in programmable nucleases, such as zinc-finger nucleases (ZFNs), transcription activator-like effector nucleases (TALENs), and clustered regularly interspaced short palindromic repeat (CRISPR)-Cas-associated nucleases. Targeted DNA alterations begin with the generation of nuclease-induced double-stranded breaks (DSBs), which lead to the stimulation of DNA recombination mechanisms in mammalian cells. Nuclease-induced DNA DSBs can be repaired by one of the two major mechanisms present in eukaryotic cells: non-homologous end joining (NHEJ) and homologous recombination (HR), resulting in gene disruptions or targeted integration, respectively. The CRISPR-Cas systems are divided into two classes based on the structural variation of the Cas genes and their organization style. Specifically, class 1 CRISPR-Cas systems consist of multiprotein effector complexes, where class 2 systems includes only a single effector protein; at least six CRISPR-Cas types and 29 subtypes are known presently. The most frequently used subtype of CRISPR system is the type 2 CRISPR/Cas9 system, which depends on a single Cas protein from Streptococcus pyogenes (SpCas9) targeting DNA sequences. A single-stranded guide RNA (sgRNA) and a Cas9 endonuclease form a targeting complex, wherein the sgRNA binds to the target sequence and Cas9 precisely cleaves the DNA to generate a DSB and subsequently activate cellular repair programs. Conveniently, changing the sgRNA sequence allows the targeting of new sites, without requiring changes to the Cas9 protein.

Specific delivery methods have been developed for targeting both Cas9 and sgRNAs directly to the organ of interest in vivo, including direct transfection, lentiviral and adeno-associated virus (AAV)-based transduction, and nanoparticle delivery. Cells may also be isolated from a patient to be treated, edited, and then re-engrafted back to the patient. Such an approach is used in the preparation of chimeric antigen receptor (CAR) T cells for cancer immunotherapy, wherein the patient's T cells are isolated, reengineered and modified with tumor-antigen-specific receptors and co-stimulating molecules, transduced with a CAR viral vector, amplified, and then infused back into the patient. Furthermore, the development of allogeneic universal “off-the-shelf” CAR-T cells has been demonstrated effectively using a one-shot CRISPR protocol to knockout endogenous TCR and HLA class 1 molecules.

Determining whether the cell of interest has been successfully targeted by a genome editing endonuclease is traditionally performed via bulk harvesting of cell lysate and analysis of total genomic material. Some of the current challenges in therapeutic targeting involve increasing the specificity of gene correction, improving the efficiency of nuclease editing, and optimizing the delivery systems. By using the in situ sequencing methods described herein, high-resolution information is obtained to decipher the effectiveness of a genome editing treatment, for example, the production of allogeneic CAR T cells.

For example, a population of T cells is subjected to a genome editing technique, for example CRISPR/Cas9, to knockout the TCR and HLA class 1 loci. The cells are then attached to a substrate surface, fixed, and permeabilized according to known methods in the art. Targeted oligonucleotide probes for the TCR and HLA class 1 loci are then annealed to the nucleic acid molecule, and the target sequence is incorporated into a circular polynucleotide as described herein. The resulting circularized oligonucleotide is primed with an amplification primer and extended with a strand-displacing polymerase to generate a concatemer containing multiple copies of the target nucleic acid sequence. The resulting concatemer may then be sequenced in situ, shedding insight into the efficacy of the genome editing technique. Additionally, using the methods described herein, one or more organelles (e.g., the Golgi Apparatus and plasma membrane) may be illuminated in one or more rounds of detection. The fluorophores may be removed from the probes, and subsequent stains or probes may be used to detect additional biomolecules. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

What is claimed is:

1. A method of analyzing a sample, the method comprising:
depositing a sample comprising a cell on a solid support;
contacting the sample with a first probe and a second probe, thereby forming a first complex and a second complex, wherein
the first complex comprises the first probe bound to a first organelle, wherein said first probe comprises a first fluorescent dye attached to the first probe via a cleavable linker;
the second complex comprises the second probe bound to a second organelle, wherein said second probe comprises a second fluorescent dye attached to the second probe via a covalent linker;
directing an excitation light to sample and detecting an emission light from the first fluorescent dye; and
cleaving the cleavable linker and the directing an excitation light to sample and detecting an emission light from the second fluorescent dye.

2. The method of claim 1, wherein the first fluorescent dye and the second fluorescent dye are spectrally distinct.

3. The method of claim 1, wherein the first fluorescent dye and the second fluorescent dye are spectrally identical.

4. The method of claim 1, wherein cleaving comprises contacting the cleavable linker with a cleaving agent.

5. The method of claim 4, wherein the cleaving agent is a reducing agent.

6. The method of claim 4, wherein the cleavable linker is a hydrazone linker, disulfide linker, azido linker, β-glucuronide linker, para-aminobenzyl alcohol (PABA) linker, phosphoramidate linker, maleimide ether linker, or triglycine (Gly-Gly-Gly) linker.

7. The method of claim 4, wherein the cleavable linker comprises two or more cleavable sites.

8. The method of claim 1, wherein the cleavable linker is a chemically cleavable linker, enzymatically cleavable linker, photo-cleavable linker.

9. The method of claim 1, wherein the first organelle is actin and the second organelle is a mitochondria.

10. The method of claim 1, wherein the first organelle is an endoplasmic reticulum and the second organelle is a mitochondria.

11. The method of claim 1, wherein the sample is frozen tissue.

12. The method of claim 1, wherein the sample is Formalin-Fixed Paraffin-Embedded tissue.

13. The method of claim 1, wherein the first probe and the second probe are each an antibody.

14. The method of claim 1, wherein the first probe comprises phalloidin, wheat germ agglutinin, concanavalin A, or annexin V.

15. The method of claim 1, further comprising detecting a nucleic acid molecule in the sample by binding a third probe to the nucleic acid molecule, wherein said third probe is an oligonucleotide, and detecting a sequence of the oligonucleotide.

16. The method of claim 15, wherein the nucleic acid molecule is outside of the cell.

17. The method of claim 1, further comprising detecting a protein in the sample by binding a third probe to the protein, wherein said third probe is an antibody-oligonucleotide conjugate, and detecting the oligonucleotide of the antibody-oligonucleotide conjugate.

18. The method of claim 1, further comprising determining the morphology of the cell based on the detected emissions.

19. A device configured to analyze a sample, said device comprising
a solid support comprising the sample, wherein the sample comprises a cell;
a first probe bound to a first organelle in the cell, wherein said first probe comprises a first fluorescent dye attached to the first probe via a cleavable linker;
a second probe bound to a second organelle in the cell, wherein said second probe comprises a second fluorescent dye attached to the second probe via a covalent linker;
a light source configured to provide excitation light having a wavelength between 200 nm to 1500 nm; and
an image sensor configured to detect fluorescent emissions from the sample;
wherein the solid support forms part of a flow cell.

20. The device of claim 19, wherein the solid support comprises polymerized units of alkoxysilyl methacrylate, alkoxysilyl acrylate, alkoxysilyl methylacrylamide, alkoxysilyl methylacrylamide, or a copolymer thereof.
